(19) United States
(12) Patent Application Publication
(10) Pub. No.: US 2008/0255153 A1
(43) Pub. Date: Oct. 16, 2008

(54) New compounds

(75) Inventors: Ulf Bremberg, Uppsala (SE); Auri Linden, Stockholm (SE); Thomas Lundback, Trangsund (SE); Jonas Nilsson, Ingaro (SE); Marie Wiik, Uppsala (SE); Magnus Bergner, Lund (SE); Peter Brandt, Uppsala (SE); Kristin Hammer, Sollentuna (SE); Rune Ringom, Uppsala (SE)

Correspondence Address: Edwards Angell Palmer & Dodge LLP, P.O. Box 55874, Boston, MA, 02205, US

(73) Assignee: BIOVITRUM AB (publ), Stockholm (SE)

(21) Appl. No.: 12/079,563

(22) Filed: Mar. 27, 2008

Related U.S. Application Data
(60) Provisional application No. 60/967,087, filed on Aug. 31, 2007.

(30) Foreign Application Priority Data
Mar. 28, 2007 (SE) 0700784-2

(51) Int. Cl. A61K 31/519; C07D 487/04; A61P 9/00

(52) U.S. Cl. 514/255.05; 544/281; 514/259.3

(57) ABSTRACT

The present invention relates to compounds of formula (I):

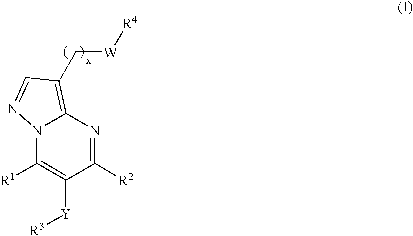

and pharmaceutically acceptable salts thereof, which are useful as inhibitors of human stearoyl-CoA desaturase (SCD). The invention further relates to pharmaceutical compositions comprising these compounds and to the use of these compounds for the treatment or prevention of medical conditions in which the modulation of SCD activity is beneficial, such as cardiovascular diseases, obesity, non-insulin-dependent diabetes mellitus, hypertension, metabolic syndrome, neurological diseases, immune disorders, cancer and various skin diseases.

### CROSS REFERENCE TO RELATED APPLICATIONS

This application claims benefit of U.S. Provisional Application No. 60/967,087, filed Aug. 31, 2007; and Swedish Application No. 0700784-2, filed Mar. 28, 2007. The entire content of each of these applications are herein incorporated by reference.

### FIELD OF THE INVENTION

The present invention relates to compounds of the formula (I), said compounds being useful as inhibitors of human stearoyl-CoA desaturase (SCD) activity. The invention further relates to the use of compounds of the formula (I) for treatment of medical conditions in which the modulation of SCD activity is beneficial, such as cardiovascular diseases, obesity, non-insulin-dependent diabetes mellitus, hypertension, metabolic syndrome, neurological diseases, immune disorders, cancer and various skin diseases.

### BACKGROUND ART

The lipid composition of cellular membranes is regulated to maintain membrane fluidity. A key enzyme involved in this process is the microsomal stearoyl-CoA desaturase (SCD; Δ9-desaturase; EC 1.14.99.5), which is the rate-limiting enzyme in the cellular synthesis of monounsaturated fatty acids from saturated fatty acids [see e.g. Ntambi (1999) J. Lipid Res. 40, 1549 for a review]. The principal products of SCD are oleoyl-CoA and palmitoleoyl-CoA, which are formed by desaturation of stearoyl-CoA and palmitoyl-CoA, respectively. A proper ratio of saturated to monounsaturated fatty acids contributes to membrane fluidity. Alterations in this ratio have been implicated in various disease states including cardiovascular disease, obesity, non-insulin-dependent diabetes mellitus, hypertension, neurological diseases, immune disorders, cancer and various skin diseases (Ntambi (1999) J. Lipid Res. 40, 1549; Sampath & Ntambi (2008) Future Lipidol. 3, 163-173). The regulation of SCD, the expression and activity of which is known to be sensitive to e.g. dietary changes and hormonal balance, is therefore of considerable physiological importance.

Several mammalian SCD genes have been cloned. Four SCD isoforms, SCD1 through SCD4, have been identified in mouse. In contrast, only two isoforms are known in rat and man. The sequence of human SCD1 from liver was first deposited in June 1997 (GenBank accession number Y13647) and the full-length cloning of human SCD1 is later described in WO 00/09754 and in Zhang et al. (1999) Biochem. J. 340, 255. The other human SCD isoform has been named SCD5 because it bears little sequence homology to alternate mouse or rat isoforms (WO 02/26944; Zhang et al. (2005) Biochem J. 388, 135; Wang et al. (2005) Biochem. Biophys. Res. Comm. 332, 735).

Early studies in rodents demonstrated that insulin as well as carbohydrate rich diets are key components in the upregulation of hepatic SCD activity [Oshino and Sato (1972) Arch. Biochem. Biophys. 149, 369; Prasad and Joshi (1979) J. Biol. Chem. 254, 997; Waters and Ntambi (1994) J. Biol. Chem. 269, 27773]. Fructose appears to play a key role in this process since this carbohydrate, contrary to glucose, not only upregulates hepatic SCD activity but also corrects the defective lipogenesis that appears in diabetic animals (see above cited references and references therein). Later studies showed that the expression of SCD1, the major SCD isoform in hepatocytes, is a crucial component in the fructose-mediated elevation of lipogenic enzymes [Miyazaki et al. (2004) J. Biol. Chem. 279, 25164], demonstrating a key role of this enzyme in hepatic lipogenesis.

There were also observations of elevated SCD activity in animal models of type 2 diabetes and obesity [see e.g. Enser (1975) Biochem. J. 148, 551; Legrand and Hermier (1992) Int. J. Obes. Relat. Metab Disord. 16, 289; Jones et al. (1996) Am. J. Physiol. 271, E44] and increased SCD activity was also shown to be associated with obesity in man [Pan et al. (1994) J. Nutr. 124, 1555], which led to descriptions of the potential role of SCD activity in type 2 diabetes and obesity amongst other diseases [Ntambi J M. (1999) J. Lipid Res. 40, 1549]. SCD1 appeared to be of primary interest based on the selective suppression of this isoform in differentiating preadipocytes by thiazolidinediones, data that were strengthened by the suppression of SCD1 in tissues of metabolic interest in vivo [Kim et al. (2000) In: Adipocyte Biology and Hormone Signaling, 27th Steenbock Symposium, Madison, Wis., June, 1999 (J. M. Ntambi, ed.), IOS Press, The Netherlands, pp. 69].

More recent studies based on animal models in which SCD1 levels are suppressed either by means of genetic ablation or by anti-sense treatment have confirmed a key role of SCD1 in the regulation of lipid synthesis versus oxidation as well as for the development of diet-induced obesity [Miyazaki et al. (2000) J. Biol. Chem. 275, 30132; WO 01/62954; Ntambi et al. (2002) Proc. Natl. Acad. Sci. USA 99, 11482; Cohen et al. (2002) Science 297, 240; Jiang et al. (2005) J. Clin. Invest. 115, 1030; Gutierrez-Juarez et al. (2006) J. Clin. Invest. 116, 1686]. The interest in SCD activity as a potential target for the development of anti-obesity treatments has thus increased significantly, prompted also by additional reports on the correlation of SCD1 activity with circulating triglyceride levels in mice as well as man [WO 01/62954; Attie et al. (2002) J. Lipid Res. 43, 1899] as well as confirming observations of elevated SCD activity in the muscles of obese people [Hulver et al. (2005) Cell Metab. 2, 251].

Besides the above described findings, both asebia mice carrying a deletion in the SCD1 gene (Zheng et al. (1999) Nature Genet. 23, 268) and SCD1 knock-out mice (Miyazaki et al. (2001) J. Nutr. 131, 2260) develop skin and eye abnormalities. These changes include hair loss as well as atrophy of the sebaceous and meibomian glands. It is therefore believed that modulation of SCD activity can be of importance in the treatment of disease states that are associated with changes in the lipid composition in these tissues and their lipid secretions as well as changes in the composition of circulating lipids that impact these tissues (see e.g. Ntambi (1999) J. Lipid Res. 40, 1549 for a general description and United States Patent 20020151018 for a more specific description). Skin diseases where it could be of relevance to apply a modulator of SCD activity include but are not restricted to e.g. essential fatty acid deficiency, eczema, acne, psoriasis and rosacea. Based on the above described phenotypes other potential applications of a SCD modulator involve a selective suppression or stimulation of hair growth (see e.g. European patent application EP1352627 A2).

It is furthermore clear for anyone skilled in the art that the desired distribution of these modulators may depend on the therapeutic indication or disease state or other application of the compounds described herein. Hence for the treatment of metabolic diseases such as type 2 diabetes and obesity, it may be desirable not to impact skin glands, hair or eyes in a negative way, i.e. such as what is observed in the above described mouse models that lack SCD1 expression. Pharmacological modulation of SCD1 activity by means of anti-sense mediated inhibition shows beneficial effects on type 2 diabetes and obesity parameters, without a negative impact on hair or skin [Jiang et al. (2005) J. Clin. Invest. 115, 1030; Gutierrez-Juarez et al. (2006) J. Clin. Invest. 116, 1686]. It is possible that this results from a reduced level of inhibition of SCD1 expression compared to the homozygous SCD1 knock-outs, but it may also be caused by the limited tissue distribution that is typically seen with anti-sense based inhibitors. On the contrary, for treatments of skin or hair diseases it may be desirable to ensure exposure in these tissues while limiting systemic exposure, such that e.g. direct application to the skin may be preferable. It is thus clear that depending on the respective tissue distribution profiles, whether caused by their intrinsic properties or by the use of various forms of administrations or formulations, SCD activity modulators will be suitable for different therapeutic indications.

The above described data serve to illustrate the validity of modulating stearoyl-CoA desaturase activity for treatment of disorders and diseases that include but are not restricted to those related to the metabolic syndrome, e.g. type 2 diabetes, obesity, non-alcoholic fatty liver disease and more. It is also described in the above cited literature that more than one isoform of SCD exists, the numbers and identities of which differ between species. The majority of findings as outlined above and in the cited references refers to SCD1, but the contributions made by SCD5 to the metabolism in man are less well understood. Depending on what disorder or disease a treatment is aimed at the modulation of the stearoyl-CoA desaturase activity may therefore involve the modulation of both or either of these activities. Consequently, there is a need for identifying molecules that modulate SCD activity and are potentially useful for the treatment of e.g. obesity, type 2 diabetes, coronary artery disease, atherosclerosis, heart disease, fatty liver diseases such as non-alcoholic steatohepatitis, cerebrovascular disease, essential fatty acid deficiency, eczema, acne, psoriasis, rosacea, or for the treatment of excessive hair growth, e.g. hirsutism.

Substituted pyrazolopyrimidine compounds are known in the art, see e.g. U.S. patent application Ser. No. 11/244,628 (Publication No. 2006/0094706). However, it has not previously been shown that such compounds are capable of modulating SCD activity.

### DISCLOSURE OF THE INVENTION

It has surprisingly been shown that compounds of the formulas herein are active as inhibitors of SCD activity. As such they are potentially useful for modulating SCD activity and thereby can serve to regulate lipid levels and composition in mammals. As such they are potentially useful in the treatment of SCD related diseases such as cardiovascular diseases, obesity, non-insulin-dependent diabetes mellitus, hypertension, metabolic syndrome, fatty liver diseases, neurological diseases, immune disorders, cancer and various skin diseases.

In a first aspect, the invention relates to a compound of formula (I),

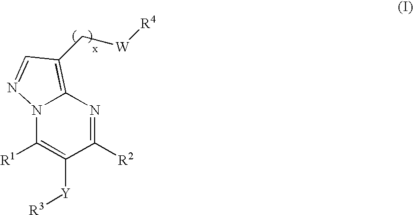

and pharmaceutically acceptable salts, solvates, hydrates, geometrical isomers, racemates, tautomers, optical isomers, or N-oxides thereof, wherein:

x is 0 or 1;

W is selected from the group consisting of a direct bond, —C(O)N(R⁵)—, —N(R⁵)C(O)—, —C(O)O—, —OC(O)—, —O—, —N(R⁵)C(O)N(R⁵)—, and —N(R⁵)—, wherein each R⁵ is independently hydrogen, C₁₋₃alkyl, or C₁₋₄-alkoxy-C₂₋₄-alkyl;

R¹ and R² are independently selected from the group consisting of hydrogen, C₁₋₃alkyl and C₁₋₃-fluoroalkyl, provided that at least one of R¹ and R² is hydrogen;

Y is selected from the group consisting of —S—, —O—, —N— and C₁₋₃-alkylene, wherein C₁₋₃-alkylene is optionally monosubstituted with hydroxy or oxo, or is partly or fully fluorinated;

R³ is aryl or heteroaryl, said aryl or heteroaryl residue being optionally substituted in one or more positions with a substituent independently selected from:
(a) halogen,
(b) C₁₋₆-alkyl,
(c) C₁₋₆-alkoxy,
(d) fluoro-C₁₋₃-alkyl,
(e) fluoro-C₁₋₃-alkoxy,
(f) C₃₋₇-cycloalkyl,
(g) C₃₋₇-cycloalkoxy,
(h) methylenedioxy,
(i) hydroxy-C₁₋₃-alkyl,
(j) cyano,
(k) hydroxy,
(l) C₁₋₆-alkylthio,
(m) fluoro-C₁₋₆-alkylthio,
(n) C₁₋₆-alkylsulfonyl,
(o) aryl-C₁₋₃-alkoxy, wherein aryl is optionally substituted in one or two positions with a substituent selected from halogen, methoxy, ethoxy, methyl, ethyl and trifluororomethyl;

R⁴ is selected from the group consisting of C₁₋₄-alkoxy-C₂₋₆-alkyl, hydroxy-C₁₋₆-alkyl, C₁₋₄-alkylthio-C₂₋₆-alkyl, cyano-C₁₋₆-alkyl, heteroarylamino-C₂₋₆-alkyl, heterocyclylamino-C₂₋₆-alkyl, heterocyclyl-C₁₋₆-alkyl, aryl-C₁₋₄-alkoxy-C₂₋₄-alkyl, dihydroxy-C₃₋₄-alkoxy-C₂₋₄-alkyl, cyano-C₁₋₄-alkoxy-C₂₋₄-alkyl, hydroxy-C₂₋₄-alkoxy-C₂₋₄-alkyl, aminocarbonyl-C₁₋₄-alkoxy-C₂₋₄-alkyl, C₁₋₄-alkoxy-C₂₋₄-alkoxy-C₂₋₄-alkyl, hydroxy-C₂₋₄-alkoxy-C₂₋₄-alkoxy-C₂₋₄-alkyl, C₂₋₄-alkenyloxy-C₂₋₆-alkyl, C₁₋₄-alkylaminocarbonyl-C₁₋₄-alkoxy-C₂₋₄-alkyl, di-(C₁₋₂-alkyl)aminocarbonyl-C₁₋₄-alkoxy-C₂₋₄-alkyl, aryl, aryl-C₁₋₆-alkyl, heteroaryl and heteroaryl-C₁₋₆-alkyl, wherein any aryl or heteroaryl residue can be optionally substituted with one or more substituents R⁸; or

R⁴ is C₁₋₆-alkylene-V—R⁶;

wherein V is selected from the group consisting of —C(O)N(R⁷)—, —C(O)O—, —OC(O)—, —C(O)—, —N(R⁷)C(O)O—, —OC(O)N(R⁷)—, —N(R⁷)C(O)—, —N(R⁷)C(O)N(R⁷)—, —S—, —S(O)—, —S(O)₂—, —S(O)N(R⁷)—, —N(R⁷)S(O)—, —S(O)₂N(R⁷)— and —N(R⁷)S(O)₂—;

and wherein each R⁶ and each R⁷ are independently selected from the group consisting of hydrogen, C₁₋₅-alkyl, C₃₋₆-cycloalkyl (optionally substituted with oxo), C₃₋₆-cycloalkyl-C₁₋₄-alkyl, hydroxy-C₁₋₄-alkyl, C₂₋₄-alkynyl, fluoro-C₁₋₅-alkyl, aryl, aryl-C₁₋₄-alkyl, heteroaryl, and heteroaryl-C₁₋₄-alkyl, wherein any aryl or heteroaryl residue can be optionally substituted with one or more substituents R⁸;

provided that when V is selected from —S(O)—, —S(O)₂—, —C(O)—, —N(R⁷)C(O)O—, —N(R⁷)S(O)—, or —N(R⁷)S(O)₂—, then R⁶ is not hydrogen;

R⁸ is independently selected from the group consisting of:
(a) C₁₋₄-alkylsulfonyl,
(b) C₁₋₄-alkylsulfinyl,
(c) C₁₋₄-alkylthio,
(d) hydroxy-C₂₋₄-alkylsulfonyl,
(e) trifluoromethylsulfonyl,
(f) —S(O)₂NR⁹R⁹,
(g) C₁₋₄-alkylsulfonamido,
(h) C₂₋₄-acylamino,
(i) C₂₋₄-acylaminomethyl,
(j) —C(O)NR⁹R⁹,
(k) —CH₂—C(O)NR⁹R⁹
(l) —NHC(O)OCH₃,
(m) C₁₋₄-alkoxy,
(n) C₃₋₅-cycloalkyloxy,
(o) —CN,
(p) —OH,
(q) C₁₋₆-alkyl
(r) hydroxy-C₁₋₂-alkyl,
(s) cyano-C₁₋₂-alkyl,
(t) C₁₋₂-alkoxy-C₁₋₂-alkyl, and
(u) halogen;

R⁹ is each independently selected from the group consisting of:
(a) hydrogen,
(b) C₁₋₃-alkyl,
(c) hydroxy-C₂₋₄-alkyl,
(d) dihydroxy-C₂₋₄-alkyl,
(e) cyano-C₁₋₃-alkyl,
(f) C₁₋₂-alkoxy-C₂₋₄-alkyl, and
(g) aminocarbonyl-C₁₋₂-alkyl.

Another embodiment of the invention relates to a compound of formula (I′),

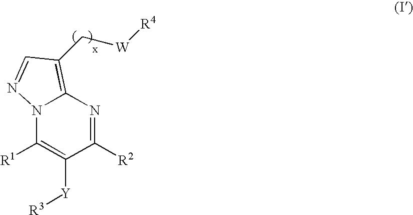

wherein x is 0 or 1;

W is selected from the group consisting of a direct bond, —C(O)N(R⁵)—, —N(R⁵)C(O)—, —C(O)O—, —OC(O)—, —N(R⁵)C(O)N(R⁵)—, and —N(R⁵)—, wherein each R⁵ is independently hydrogen, C₁₋₃-alkyl, or C₁₋₄-alkoxy-C₂₋₄-alkyl;

R¹ and R² are independently selected from the group consisting of hydrogen, C₁₋₃-alkyl and C₁₋₃-fluoroalkyl, provided that at least one of R¹ and R² is hydrogen;

Y is selected from the group consisting of —S—, —O— and C₁₋₃-alkylene, wherein C₁₋₃-alkylene is optionally monosubstituted with hydroxy or oxo, or is partly or fully fluorinated;

R³ is aryl or heteroaryl, said aryl or heteroaryl residue being optionally substituted in one or more positions with a substituent independently selected from:
(a) halogen,
(b) C_{1 6}-alkyl,
(c) C₁₋₆-alkoxy,
(d) fluoro-C₁₋₃-alkoxy,
(e) fluoro-C₁₋₃-alkyl,
(f) methylenedioxy,
(g) hydroxy-C₁₋₃-alkyl,
(h) cyano,
(i) hydroxy,
(j) C₁₋₆-alkylthio,
(k) C₁₋₆-alkylsulfonyl,
(l) aryl-C₁₋₃-alkoxy, wherein aryl is optionally substituted in one or two positions with a substituent selected from halogen, methoxy, ethoxy, methyl, ethyl and trifluororomethyl;

R⁴ is selected from the group consisting of C₁₋₄-alkoxy-C₂₋₆-alkyl, hydroxy-C₁₋₆-alkyl, C₁₋₄-alkylthio-C₂₋₆-alkyl, cyano-C₁₋₆-alkyl, heteroarylamino-C₂₋₆-alkyl, heterocyclylamino-C₂₋₆-alkyl, heterocyclyl-C₁₋₆-alkyl, aryl-C₁₋₄-alkoxy-C₂₋₄-alkyl, dihydroxy-C₃₋₄-alkoxy-C₂₋₄-alkyl, cyano-C₁₋₄-alkoxy-C₂₋₄-alkyl, hydroxy-C₂₋₄-alkoxy-C₂₋₄-alkyl, aminocarbonyl-C₁₋₄-alkoxy-C₂₋₄-alkyl, C₁₋₄-alkoxy-C₂₋₄-alkoxy-C₂₋₄-alkyl, hydroxy-C₂₋₄-alkoxy-C₂₋₄-alkoxy-C₂₋₄-alkyl, C₂₋₄-alkenyloxy-C₂₋₆-alkyl, C₁₋₄-alkylaminocarbonyl-C₁₋₄-alkoxy-C₂₋₄-alkyl and di-(C₁₋₂-alkyl)aminocarbonyl-C₁₋₄-alkoxy-C₂₋₄-alkyl; or

R⁴ is C₁₋₆-alkylene-V—R⁶;

wherein V is selected from the group consisting of —C(O)N(R⁷)—, —C(O)O—, —OC(O)—, —C(O)—, —N(R⁷)C(O)O)—, —OC(O)N(R⁷)—, —N(R⁷)C(O)—, —N(R⁷)C(O)N(R⁷)—, —S(O)—, —S(O)₂—, —S(O)N(R⁷)—, —N(R⁷)S(O)—, —S(O)₂N(R⁷)— and —N(R⁷)S(O)₂—;

and wherein each R⁶ and each R⁷ are independently selected from the group consisting of hydrogen, C₁₋₅-alkyl, C₃₋₆-cycloalkyl (optionally substituted with oxo), C₃₋₆-cycloalkyl-C₁₋₄-alkyl, hydroxy-C₁₋₄-alkyl, C₂₋₄-alkynyl, aryl (optionally substituted with halogen, methoxy, trifluoromethyl and methyl), heteroaryl and fluoro-C₁₋₅-alkyl;

provided that when V is selected from —S(O)—, —S(O)₂—, —C(O)—, —N(R⁷)C(O)—, —N(R⁷)S(O)—, or —N(R⁷)S(O)₂—, then R⁶ is not hydrogen.

In a preferred embodiment of the invention, W is selected from the group consisting of —C(O)N(R⁵)—, —N(R⁵)C(O)—, —C(O)O—, —OC(O)—, —N(R⁵)C(O)N(R⁵)— and —N(R⁵)—, wherein each R⁵ is independently hydrogen, C₁₋₃-alkyl, or C₁₋₄-alkoxy-C₂₋₄-alkyl.

In another preferred embodiment, Y is methylene, 1,1-ethylene or —S—, and R³ is aryl, which is optionally substituted in one or more positions with a substitutent selected from halogen, C₁₋₆-alkyl, C₁₋₆-alkoxy, fluoro-C₁₋₃-alkoxy and fluoro-C₁₋₃-alkyl.

In yet another preferred embodiment, R¹ is C₁₋₃-alkyl and R² is H, or R¹ is H and R² is C₁₋₃-alkyl, or R¹ and R² are H;

More preferred compounds of the invention include those wherein:

x is 0 and W is —C(O)NH—, —NHC(O)—, —C(O)O— or —NHC(O)NH—;

Y is methylene, 1,1-ethylene or —S—; and

R¹ is methyl and R² is H, or

R¹ is H and R² is methyl, or

R¹ and R² are each H;

R³ is aryl, optionally substituted in one or more positions with a substituent independently selected from the group R¹⁰ consisting of fluoro, chloro, bromo, methyl, ethyl, methoxy, ethoxy, trifluoromethyl, trifluoromethoxy and methylthio;

R⁴ is selected from the group consisting of C₁₋₄-alkoxy-C₂₋₄-alkyl, hydroxy-C₁₋₄-alkyl, C₁₋₄-alkylthio-C₂₋₄-alkyl, cyano-C₁₋₄-alkyl, heteroaryl, heteroaryl-C₁₋₄-alkyl, heteroaryl-amino-C₂₋₄-alkyl, heterocyclyl-C₁₋₄-alkyl, aryl-C₁₋₄-alkoxy-C₂₋₄-alkyl, dihydroxy-C₃₋₄-alkoxy-C₂₋₄-alkyl, cyano-C₁₋₄-alkoxy-C₂₋₄-alkyl, aminocarbonyl-C₁₋₄-alkoxy-C₂₋₄-alkyl, hydroxy-C₂₋₄-alkoxy-C₂₋₄-alkyl, C₁₋₄-alkoxy-C₂₋₄-alkoxy-C₂₋₄-alkyl, hydroxy-C₂₋₄-alkoxy-C₂₋₄-alkoxy-C₂₋₄-alkyl, C₂₄-alkenyloxy-C₂₋₄-alkyl, C₁₋₄-alkylaminocarbonyl-C ₁₋₄-alkoxy-C₂₋₄-alkyl and di-(C₁₋₂-alkyl)aminocarbonyl-C₁₋₄-alkoxy-C₂₋₄-alkyl, wherein any aryl or heteroaryl residue can be optionally substituted with one or more substituents R⁸ (as defined above for formula (I)); or

R⁴ is C₁₋₄-alkylene-V—R⁶;

wherein V is selected from the group consisting of —C(O)N(R⁷)—, —N(R⁷)C(O)—, —C(O)—, —S—, —S(O)—, —S(O)₂—, —S(O)N(R⁷)—, —N(R⁷)S(O)—, —S(O)₂N(R⁷)—, and —N(R⁷)S(O)₂—;

wherein each R⁶ is independently selected from the group consisting of hydrogen, C₁₋₅-alkyl, C₃₋₆-cycloalkyl (optionally substituted with oxo), C₃₋₆-cycloalkyl-C₁₋₄-alkyl, hydroxy-C₁₋₄-alkyl, C₂₋₄-alkynyl, aryl, heteroaryl, heteroaryl-C₁₋₄-alkyl and fluoro-C₁₋₅-alkyl; wherein any aryl or heteroaryl residue can be optionally substituted with one or more substituents R⁸;

and wherein each R⁷ is independently selected from the group consisting of hydrogen and C₁₋₃-alkyl;

provided that when V is selected from —S(O)—, —S(O)₂—, —C(O)—, —N(R⁷)S(O)— or —N(R⁷)S(O)₂—, then R⁶ is not hydrogen.

Further, when R⁴ is selected from C₁₋₄-alkylene-V—R⁶, said C₁₋₄-alkylene-V—R⁶ more preferably represents a group selected from the group consisting of C₁₋₅-acylamino-C₂₋₄-alkyl, aminocarbonyl-C₁₋₄-alkyl, hydroxy-C₁₋₄-alkylcarbonylamino-C₂₋₄-alkyl, C₂₋₄-alkynylcarbonylamino-C₂₋₄-alkyl, C₁₋₄-alkylaminocarbonyl-C₁₋₄-alkyl, di-(C₁₋₂-alkyl)-aminocarbonyl-C₁₋₄-alkyl, C₁₋₄-alkylsulfinyl-C₁₋₄-alkyl, C₁₋₄-alkylsulfonyl-C₁₋₄-alkyl, heteroarylcarbonylamino-C₂₋₄-alkyl, arylcarbonylamino-C₂₋₄-alkyl, hydroxy-C₁₋₄-alkylaminocarbonyl-C₁₋₄-alkyl, C₁₋₄-alkylaminosulfinyl-C₁₋₄-alkyl, C₁₋₄-alkylamino-sulfonyl-C₁₋₄-alkyl, C₁₋₄-alkylsulfinamido-C₂₋₄-alkyl, C₁₋₄-alkylsulfonamido-C₂₋₄-alkyl, C₂₋₅-acyl-C₁₋₄-alkyl, C₃₋₆-cycloalkylcarbonyl-C₁₋₄-alkyl and C₃₋₆-cycloalkyl-C₁₋₄-alkyl-carbonyl-C₁₋₄-alkyl, wherein any aryl or heteroaryl residue can be optionally substituted with one or more substituents R⁸ (as defined above for formula (I));

In more preferred compounds of the invention, R³ is phenyl which is optionally substituted in one, two or three positions, and even more preferably in one or two positions, with a substituent independently selected from the group R¹⁰ as defined above.

In particularly preferred compounds of the invention, R³ is selected from the group consisting of phenyl, 3-bromophenyl, 4-bromophenyl, 3-trifluoromethylphenyl, 3-trifluoro-methoxyphenyl, 3,4-dichlorophenyl, 2,3-dichlorophenyl, 2,4-dichlorophenyl, 2,5-dichloro-phenyl, 4-chloro-2-fluorophenyl, 3-chloro-4-fluorophenyl, 5-chloro-2-fluorophenyl, 2-methyl-5-trifluoromethylphenyl, 4-chloro-3-trifluoromethylphenyl, 4-fluoro-3-trifluoro-methylphenyl, 4-chloro-3-trifluoromethoxyphenyl, 4-fluoro-3-trifluoromethoxyphenyl, 5-chloro-2-trifluoromethylphenyl, and 2-chloro-5-trifluoromethylphenyl;

R⁴ is selected from the group consisting of 2-methoxyethyl, 2-hydroxyethyl, 3-methoxypropyl, 3-hydroxypropyl, 2-(2-hydroxyethoxy)ethyl, 2-(2-aminocarbonylethoxy)-ethyl, cyanomethyl, 2-(2-cyanoethoxy)ethyl, 2-(2-hydroxy-2-methylpropoxy)ethyl, 2-(formylamino)ethyl, 2-(acetylamino)ethyl, 2-(propionylamino)ethyl, 2-(ethynylcarbonyl-amino)ethyl, aminocarbonylmethyl, methylaminocarbonylmethyl, 2-(aminocarbonyl)ethyl, 2-(hydroxymethylcarbonylamino)ethyl, 2-(methylsulfinyl)ethyl, 2-(methylsulfonyl)ethyl, 2-(dimethylamino)-2-oxoethyl, 2-(benzyloxy)ethyl, tetrahydrofuran-2-ylmethyl, 2-[(1H-pyrrol-2-ylcarbonyl)amino]ethyl, 2-furylmethyl, 2-(2-furyl)ethyl, 2-[(2-furylmethyl)-thio]ethyl, 2-(pyridin-2-ylamino)ethyl, 6-methoxypyridin-3-yl, 2-[(pyrazin-2-ylcarbonyl)-amino]ethyl, 2-(isonicotinoylamino)ethyl, pyridin-3-yl, [6-(hydroxymethyl)pyridin-2-yl]-methyl and 2-(2,3-dihydroxypropoxy)ethyl.

Particularly preferred compounds of the invention are the compounds selected from the group consisting of:
tert-butyl[2-({[6-(3,4-dichlorobenzyl)pyrazolo[1,5-α]pyrimidin-3-yl]carbonyl}amino)-ethyl]carbamate;
6-(3,4-dichlorobenzyl)-N-{2-[(pyrazin-2-ylcarbonyl)amino]ethyl}pyrazolo[1,5-α]-pyrimidine-3-carboxamide;
6-(3,4-dichlorobenzyl)-N-[2-(methylsulfinyl)ethyl]pyrazolo[1,5-α]pyrimidine-3-carboxamide;
6-(3,4-dichlorobenzyl)-N-[2-(methylsulfonyl)ethyl]pyrazolo[1,5-α]pyrimidine-3-carboxamide;
6-(3,4-dichlorobenzyl)-N-[2-(dimethylamino)-2-oxoethyl]pyrazolo[1,5-α]pyrimidine-3-carboxamide;
6-(3,4-dichlorobenzyl)-N-[2-(methylamino)-2-oxoethyl]pyrazolo[1,5-α]pyrimidine-3-carboxamide;
N-[2-(benzyloxy)ethyl]-6-(3,4-dichlorobenzyl)pyrazolo[1,5-α]pyrimidine-3-carboxamide;
6-(3,4-dichlorobenzyl)-N-(3-methoxypropyl)pyrazolo[1,5-α]pyrimidine-3 -carboxamide;
6-(3,4-dichlorobenzyl)-N-(3-hydroxypropyl)pyrazolo[1,5-α]pyrimidine-3-carboxamide;
6-(3,4-dichlorobenzyl)-N-(tetrahydrofuran-2-ylmethyl)pyrazolo[1,5-α]pyrimidine-3 -carboxamide;
6-(3,4-dichlorobenzyl)-N-[2-(isonicotinoylamino)ethyl]pyrazolo[1,5-α]pyrimidine-3-carboxamide;
6-(3,4-dichlorobenzyl)-N-[2-(pyridin-2-ylamino)ethyl]pyrazolo[1,5-α]pyrimidine-3-carboxamide;
6-(3,4-dichlorobenzyl)-N-[2-(2-furyl)ethyl]pyrazolo[1,5-α]pyrimidine-3-carboxamide;
6-(3,4-dichlorobenzyl)-N-{2-[(2-furylmethyl)thio]ethyl}pyrazolo[1,5-α]pyrimidine-3-carboxamide;
N-(3-amino-3-oxopropyl)-6-(3,4-dichlorobenzyl)pyrazolo[1,5-α]pyrimidine-3-carboxamide;
N-(2-amino-2-oxoethyl)-6-(3,4-dichlorobenzyl)pyrazolo[1,5-α]pyrimidine-3-carboxamide;
6-(4-bromobenzyl)-N-[2-(propionylamino)ethyl]pyrazolo[1,5-α]pyrimidine-3-carboxamide;
6-(4-bromobenzyl)-N-{2-[(1H-pyrrol-2-ylcarbonyl)amino]ethyl}pyrazolo[1,5-α]-pyrimidine-3 -carboxamide;
6-(4-bromobenzyl)-N-(2-hydroxyethyl)pyrazolo[1,5-α]pyrimidine-3-carboxamide;
6-(4-bromobenzyl)-N-[2-(2,3-dihydroxypropoxy)ethyl]pyrazolo[1,5-α]pyrimidine-3-carboxamide;
6-(4-bromobenzyl)-N-(2-methoxyethyl)pyrazolo[1,5-α]pyrimidine-3-carboxamide;
N-[2-(3-amino-3-oxopropoxy)ethyl]-6-(4-bromobenzyl)pyrazolo[1,5-α]pyrimidine-3-carboxamide;
6-(4-bromobenzyl)-N-[2-(2-cyanoethoxy)ethyl]pyrazolo[1,5-α]pyrimidine-3-carboxamide;
6-(4-bromobenzyl)-N-[2-(2-hydroxy-2-methylpropoxy)ethyl]pyrazolo[1,5-α]-pyrimidine-3 -carboxamide;
6-(4-bromobenzyl)-N-[2-(formylamino)ethyl]pyrazolo[1,5-α]pyrimidine-3-carboxamide;
6-(4-bromobenzyl)-N-[2-(glycoloylamino)ethyl]pyrazolo[1,5-α]pyrimidine-3-carboxamide;
6-(3-chloro-4-fluorobenzyl)-N-{[6-(hydroxymethyl)pyridin-2-yl]methyl}pyrazolo[1,5-α]pyrimidine-3-carboxamide;
N-(2-amino-2-oxoethyl)-6-(3 -chloro-4-fluorobenzyl)pyrazolo[1,5-α]pyrimidine-3-carboxamide;
N-(3-amino-3-oxopropyl)-6-(3-chloro-4-fluorobenzyl)pyrazolo[1,5-α]pyrimidine-3-carboxamide;
6-(3-chloro-4-fluorobenzyl)-N-[2-(2-cyanoethoxy)ethyl]pyrazolo[1,5-α]pyrimidine-3-carboxamide;
6-[4-chloro-3-(trifluoromethoxy)benzyl]-N-(2-hydroxyethyl)pyrazolo[1,5-α]pyrimidine-3-carboxamide;
N-(3 -amino-3 -oxopropyl)-6-[4-chloro-3-(trifluoromethoxy)benzyl]pyrazolo[1,5-α]-pyrimidine-3-carboxamide;
N-(2-amino-2-oxoethyl)-6-[4-chloro-3-(trifluoromethyl)benzyl]pyrazolo[1,5-α]-pyrimidine-3-carboxamide;
N-[2-(acetylamino)ethyl]-6-[4-fluoro-3-(trifluoromethyl)benzyl]pyrazolo[1,5-α]-pyrimidine-3 -carboxamide;
N-(2-amino-2-oxoethyl)-6-[4-fluoro-3-(trifluoromethyl)benzyl]pyrazolo[1,5-α]-pyrimidine-3-carboxamide;
N-(3-amino-3-oxopropyl)-6-{1-[3-(trifluoromethyl)phenyl]ethyl}pyrazolo[1,5-α]-pyrimidine-3-carboxamide;
6-benzyl-N-[2-(2-hydroxyethoxy)ethyl]-5-methylpyrazolo[1,5-α]pyrimidine-3-carboxamide;
6-[4-fluoro-3-(trifluoromethoxy)benzyl]-N-[2-(2-hydroxyethoxy)ethyl]-5-methyl-pyrazolo[1,5-α]pyrimidine-3-carboxamide;
6-benzyl-N-[2-(2-hydroxyethoxy)ethyl]-7-methylpyrazolo[1,5-α]pyrimidine-3-carboxamide;
N-[2-(2-hydroxyethoxy)ethyl]-7-methyl-6-[3-(trifluoromethyl)benzyl]pyrazolo[1,5-α]-pyrimidine-3-carboxamide;
N-(3-amino-3-oxopropyl)-7-methyl-6-[3-(trifluoromethoxy)benzyl]pyrazolo[1,5-α]-pyrimidine-3-carboxamide;
N-[2-(acetylamino)ethyl]-6-[4-chloro-3-(trifluoromethoxy)benzyl]-7-methylpyrazolo-[1,5-α]pyrimidine-3-carboxamide;
6-[4-chloro-3-(trifluoromethoxy)benzyl]-N-[2-(2-hydroxyethoxy)ethyl]-7-methyl-pyrazolo[1,5-α]pyrimidine-3-carboxamide;
N-[2-(acetylamino)ethyl]-6-[4-fluoro-3-(trifluoromethoxy)benzyl]-7-methylpyrazolo-[1,5-α]pyrimidine-3-carboxamide;
6-[4-fluoro-3-(trifluoromethyl)benzyl]-N-(6-methoxypyridin-3-yl)pyrazolo[1,5-α]-pyrimidine-3-carboxamide;
6-[4-fluoro-3-(trifluoromethyl)benzyl]-N-pyridin-3-ylpyrazolo[1,5-α]pyrimidine-3-carboxamide;
2-(acetylamino)ethyl 6-[4-fluoro-3-(trifluoromethyl)benzyl]pyrazolo[1,5-α]pyrimidine-3-carboxylate;
2-amino-2-oxoethyl 6-[4-fluoro-3-(trifluoromethyl)benzyl]pyrazolo[1,5-α]pyrimidine-3-carboxylate;
2-(2-hydroxyethoxy)ethyl 6-[4-fluoro-3-(trifluoromethyl)benzyl]pyrazolo[1,5-α]-pyrimidine-3-carboxylate;
N-(2-amino-2-oxoethyl)-6-{[3-(trifluoromethyl)phenyl]thio}pyrazolo[1,5-α]pyrimidine-3-carboxamide;
2-cyano-N-[6-(3,4-dichlorobenzyl)pyrazolo[1,5-α]pyrimidin-3-yl]acetamide;
N-[6-(3,4-dichlorobenzyl)pyrazolo[1,5-α]pyrimidin-3-yl]-N′-(2-furylmethyl)urea;
N-(2-amino-2-oxoethyl)-6-(2,5-dichlorobenzyl)pyrazolo[1,5-α]pyrimidine-3-carboxamide;
N-(2-amino-2-oxoethyl)-6-[5-chloro-2-(trifluoromethyl)benzyl]pyrazolo[1,5-α]-pyrimidine-3-carboxamide;
N-(2-amino-2-oxoethyl)-6-[2-chloro-5-(trifluoromethyl)benzyl]pyrazolo[1,5-α]-pyrimidine-3-carboxamide;
N-(2-amino-2-oxoethyl)-6-(2,3-dichlorobenzyl)pyrazolo[1,5-α]pyrimidine-3-carboxamide;
N-(2-amino-2-oxoethyl)-6-(4-chloro-2-fluorobenzyl)pyrazolo[1,5-α]pyrimidine-3-carboxamide;
N-(2-amino-2-oxoethyl)-6-(5-chloro-2-fluorobenzyl)pyrazolo[1,5-α]pyrimidine-3-carboxamide;
N-(2-amino-2-oxoethyl)-6-[2-methyl-5-(trifluoromethyl)benzyl]pyrazolo[1,5-α]-pyrimidine-3-carboxamide; and
N-(2-amino-2-oxoethyl)-6-(2,4-dichlorobenzyl)pyrazolo[1,5-α]pyrimidine-3-carboxamide.

In a further aspect, the invention relates to a compound of formula (I) (reference to “formula (I)″ includes formulae I, I′, etc.) for use in therapy. Said compounds are useful as modulators of stearoyl-CoA desaturase activity and as modulators of lipid composition and levels. They are preferably useful as modulators of human stearoyl-CoA desaturase activity and as modulators of lipid composition and levels in man. The invention relates in particular to a compound of formula (I) for use in the treatment or prevention of cardiovascular diseases, obesity, non-insulin-dependent diabetes mellitus, hypertension, metabolic syndrome, neurological diseases (such as Alzheimer's disease and multiple sclerosis), immune disorders (including, but not restricted to, ophtalmopathies such as Graves' Ophthalmopathy, hepatitis, alcoholic hepatitis, sinusitis, asthma, pancreatitis, osteoarthrities, rheumatoid arthrities and other autoimmune diseases), cancer (including, but not restricted to, hyperproliferative diseases with dysregulated SCD activity, i.e. malignancies, metastasis, hepatomes and the like), essential fatty acid deficiency, eczema, acne, psoriasis, rosacea, or in the treatment of excessive hair growth, e.g. hirsutism. In another aspect, the invention relates to the use of a compound of formula (I) in the manufacture of a modulator of stearoyl-CoA desaturase activity. The invention relates in particular to the use of a compound of formula (I) in the manufacture of a medicament for the treatment or prevention of cardiovascular diseases, obesity, non-insulin-dependent diabetes mellitus, hypertension, metabolic syndrome, neurological diseases (such as Alzheimer's disease and multiple sclerosis), immune disorders (including, but not restricted to, ophtalmopathies such as Graves' Ophthalmopathy, hepatitis, alcoholic hepatitis, sinusitis, asthma, pancreatitis, osteoarthrities, rheumatoid arthrities and other autoimmune diseases), cancer (including, but not restricted to, hyperproliferative diseases with dysregulated SCD activity, i.e. malignancies, metastasis, hepatomes and the like), essential fatty acid deficiency, eczema, acne, psoriasis, rosacea, or for the treatment of excessive hair growth, e.g. hirsutism.

In yet another aspect, the invention relates to a method for the modulation of stearoyl-CoA desaturase activity, which comprises administering to a mammal in need of such treatment an effective amount of a compound of formula (I). The invention relates in particular to a method for treatment of prevention of cardiovascular diseases, obesity, non-insulin-dependent diabetes mellitus, hypertension, metabolic syndrome, neurological diseases (such as Alzheimer's disease and multiple sclerosis), immune disorders (including, but not restricted to, ophtalmopathies such as Graves' Ophthalmopathy, hepatitis, alcoholic hepatitis, sinusitis, asthma, pancreatitis, osteoarthrities, rheumatoid arthrities and other autoimmune diseases), cancer (including, but not restricted to, hyperproliferative diseases with dysregulated SCD activity, i.e. malignancies, metastasis, hepatomes and the like), essential fatty acid deficiency, eczema, acne, psoriasis, rosacea, or for the treatment of excessive hair growth, e.g. hirsutism, which comprises administering to a mammal in need of such treatment an effective amount of a compound of formula (I).

In one aspect, the mammal to be treated according to the method of the present invention is man. In another aspect, the mammal to be treated according to the method of the present invention is any other mammal. Non-limiting examples of other mammals include horses, cows, sheep, goats, dogs, cats, guinea pigs, rats and other equine, bovine, ovine, canine, feline and rodent species.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabelling/assay techniques, blotting/chemiluminescence methods, real-time PCR, and the like.

### DEFINITIONS

The following definitions shall apply throughout the specification and the appended claims.

Unless otherwise stated or indicated, the term “C₁₋₆-alkyl” denotes a straight or branched alkyl group having from 1 to 6 carbon atoms. Examples of said C₁₋₆-alkyl include methyl, ethyl, n-propyl, isopropyl, n-butyl, isobutyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc.

Unless otherwise stated, “fluoro-C₁₋₆-alkyl” means a C₁₋₆-alkyl group as defined above substituted by one or more fluorine atoms. Examples of said fluoro-C₁₋₆-alkyl include 2-fluoroethyl, fluoromethyl, trifluoromethyl, 2,2,2-trifluoroethyl and 3-fluoropropyl.

Unless otherwise stated or indicated, the term “hydroxy-C₁₋₆-alkyl” denotes a C₁₋₆-alkyl group as defined above substituted with a hydroxy group. Examples of said hydroxy-C₁₋₆-alkyl include hydroxymethyl, 2-hydroxyethyl, 2-hydroxypropyl, 3-hydroxypropyl and 2-hydroxy-2-methylpropyl.

Unless otherwise stated or indicated, the term “C₁₋₆-alkylene” denotes a straight or branched divalent saturated hydrocarbon chain having from 1 to 6 carbon atoms. Examples of alkylene diradicals include methylene [—CH₂—], 1,2-ethylene[—CH₂—CH₂—],1,1-ethylene [—CH(CH₃)—], 1,2-propylene[—CH₂CH(CH₃—], 1,3-propylene[—CH₂—CH₂—CH₂—] and 1,4-butylene[—CH₂—CH₂—CH₂—CH₂—]. The alkylene groups may be optionally substituted. Optional substituents on alkylene are defined elsewhere in the specification and appended claims.

Unless otherwise stated or indicated, the term “C₁₋₆-alkoxy” refers to a group C₁₋₆-alkyl as defined above, which is attached to the remainder of the molecule through an oxygen atom. Examples of said C₁₋₆-alkoxy include methoxy, ethoxy, n-propoxy, isopropoxy, n-butoxy, isobutoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, “fluoro-C₁₋₃-alkoxy” means a C₁₋₃-alkoxy group as defined above, substituted by one or more fluorine atoms. Examples of said fluoro-C₁₋₃-alkoxy include trifluoromethoxy, difluoromethoxy, monofluoromethoxy, 2-fluoroethoxy, 2,2,2-trifluoroethoxy and 1,1,2,2-tetrafluoroethoxy.

Unless otherwise stated or indicated, the term “C₁₋₆-alkylthio” refers to a group C₁₋₆-alkyl as defined above, which is attached to the remainder of the molecule through a sulfur atom. Examples of said C₁₋₆-alkylthio include methylthio, ethylthio, n-propylthio, isopropylthio, n-butylthio, isobutylthio, sec-butylthio, t-butylthio and straight- and branched-chain pentylthio and hexylthio. For parts of the range “C₁₋₆-alkylthio” all subgroups thereof are contemplated such as C₁₋₅-alkylthio, C₁₋₄-alkylthio, C₁₋₃-alkylthio, C₁₋₂-alkylthio, C₂₋₆-alkylthio, C₂₋₅-alkylthio, C₂₋₄-alkylthio, C₂₋₃-alkylthio, C₃₋₆-alkylthio, C₄₋₅-alkylthio, etc.

Unless otherwise stated or indicated, the term “fluoro-C₁₋₆-alkylthio” refers to a C₁₋₆-alkylthio group as defined above, substituted by one or more fluorine atoms. Examples of said fluoro-C₁₋₆-alkylthio include trifluoromethylthio and difluoromethylthio.

Unless otherwise stated or indicated, the term “C₁₋₄-alkoxy-C₂₋₆-alkyl” denotes a C₁₋₄-alkoxy group, as defined above, attached to an alkyl group, as defined above, having from 2 to 6 carbon atoms. Examples of said C₁₋₄-alkoxy-C₂₋₆-alkyl include 2-methoxyethyl, 2-ethoxyethyl and 2-isopropoxyethyl.

Unless otherwise stated or indicated, the term “C₁₋₄-alkylthio-C₂₋₆-alkyl” denotes a C₁₋₄-alkylthio group, as defined above, attached to an alkyl group, as defined above, having from 2 to 6 carbon atoms. Examples of said C₁₋₄-alkylthio-C₂₋₆-alkyl include 2-methylthioethyl, 2-ethylthioethyl and 2-isopropylthioethyl.

Unless otherwise stated or indicated, the term “C₁₋₄-alkylsulfinyl” refers to a group C₁₋₄-alkyl-(SO)—.

Unless otherwise stated or indicated, the term “C₁₋₄-alkylsulfinyl-C₁₋₄-alkyl” denotes a C₁₋₄-alkylsulfinyl group, as defined herein, attached to an alkyl group, as defined above, having from 1 to 4 carbon atoms. Examples of said C₁₋₄-alkylsulfinyl-C₁₋₄-alkyl include 2-methylsulfinylethyl, 2-ethylsulfinylethyl and 2-isopropylsulfinylethyl.

Unless otherwise stated or indicated, the term “C₁₋₄-alkylsulfonyl” refers to a group C₁₋₄-alkyl-(SO₂)—.

Unless otherwise stated or indicated, the term “C₁₋₄-alkylsulfonyl-C₁₋₄-alkyl” denotes a C₁₋₄-alkylsulfonyl group, as defined herein, attached to an alkyl group, as defined above, having from 1 to 4 carbon atoms. Examples of said C₁₋₄-alkylsulfonyl-C₁₋₄-alkyl include 2-methylsulfonylethyl, 2-ethylsulfonylethyl and 2-isopropylsulfonylethyl.

Unless otherwise stated or indicated, the term “dihydroxy-C₃₋₄-alkoxy” refers to a C₃₋₄-alkoxy group which is disubstituted with hydroxy.

Unless otherwise stated or indicated, the term “dihydroxy-C₃₋₄-alkoxy-C₂₋₄-alkyl” denotes a dihydroxy-C₃₋₄-alkoxy group, as defined above, attached to an alkyl group, as defined above, having from 2 to 4 carbon atoms. Exemplary dihydroxy-C₃₋₄-alkoxy-C₂₋₄-alkyl groups include 2-(2,3-dihydroxypropoxy)ethyl and 2-(2,3-dihydroxybutoxy)ethyl.

Unless otherwise stated or indicated, the term “C₁₋₆-acyl” refers to the radical R^{a}(C═O)—, wherein R^{a} is selected from hydrogen or an alkyl group, as defined above, having from 1 to 5 carbon atoms, bonded to a carbonyl group. For parts of the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₃₋₆-acyl, C₄₋₅-acyl, etc. Exemplary acyl groups include formyl (i.e., C₁-acyl), acetyl (i.e., C₂-acyl), propanoyl, butanoyl, pentanoyl and hexanoyl.

Unless otherwise stated or indicated, the term “cyano-C₁₋₆-alkyl” denotes a C₁₋₆-alkyl group, as defined above, substituted with a cyano group. Exemplary cyano-C₁₋₆-alkyl groups include 2-cyanoethyl and 3-cyanopropyl.

Unless otherwise stated or indicated, the term “cyano-C₁₋₄-alkoxy” denotes a C₁₋₄-alkoxy group, as defined above, wherein the alkyl portion is substituted with a cyano group.

Unless otherwise stated or indicated, the term “cyano-C₁₋₄-alkoxy-C₂₋₄-alkyl” refers to a cyano-C₁₋₄-alkoxy group, as defined above, attached to a C₂₋₄-alkyl group as defined above. Exemplary cyano-C₁₋₄-alkoxy-C₂₋₄-alkyl groups include 2-(2-cyanoethoxy)ethyl and 3-(2-cyanoethoxy)propyl.

Unless otherwise stated or indicated, the term “C₂₋₄-alkenyl” denotes a straight or branched hydrocarbon chain radical containing at least one carbon-carbon double bond and having from 2 to 4 carbon atoms. Examples of said C₂₋₄-alkenyl groups include ethenyl (i.e., vinyl), 1-propenyl, 2-propenyl, 1-butenyl, 2-butenyl, and 1-methylprop-2-en-1-yl.

Unless otherwise stated or indicated, the term “C₂₋₄-alkenyloxy-C₂₋₆-alkyl” means a C₂₋₄-alkenyl-O—C₂₋₆-alkyl group wherein the C₂₋₆-alkyl and C₂₋₄-alkenyl groups are as defined herein. Exemplary C₂₋₄-alkenyloxy-C₂₋₆-alkyl groups include 2-(vinyloxy)ethyl and 2-(2-propenyloxy)ethyl.

Unless otherwise stated or indicated, the term “aryl” refers to a hydrocarbon ring system of one, two, or three, preferably one or two, rings, comprising at least one aromatic ring and having from 6-1-4, preferably 6-10, carbon atoms. Examples of aryl groups are phenyl, indenyl, indanyl (i.e., 2,3-dihydroindenyl), 1,2,3,4-tetrahydronaphthyl, 1-naphthyl, 2-naphthyl, fluorenyl and anthryl. An aryl group can be linked to the remainder of the molecule through any available ring carbon whether the ring carbon is in an aromatic ring or in a partially saturated ring. The aryl groups may be optionally substituted (e.g., with 1-10 substituents if multicyclic; 1-4 substituents if monocyclic). Optional substituents on aryl are defined elsewhere in the specification and appended claims.

Unless otherwise stated or indicated, the term “arylcarbonyl” refers to an aryl group attached to a carbonyl group, i.e., aryl-(C═O)—.

Unless otherwise stated or indicated, the term “aryl-C₁₋₄-alkoxy” denotes a C₁₋₄-alkoxy group as defined above wherein the alkyl portion is substituted with an aryl group. Exemplary aryl-C₁₋₄-alkoxy groups include benzyloxy, 2-phenylethoxy, 1-phenylethoxy or 3-phenylpropoxy. The aryl-C₁₋₄-alkoxy groups may be optionally substituted. Optional substituents on aryl-C₁₋₄-alkoxy are defined elsewhere in the specification and appended claims.

Unless otherwise stated or indicated, the term “aryl-C₁₋₄-alkoxy-C₂₋₄-alkyl” refers to an aryl-C₁₋₄-alkoxy group, as defined above, attached to a C₂₋₄-alkyl group as defined above. Exemplary aryl-C₁₋₄-alkoxy-C₂₋₄-alkyl groups include 2-benzyloxyethyl and 2-(2-phenyl-ethoxy)ethyl.

Unless otherwise stated or indicated, the term “C₁₋₅-acylamino-C₂₋₄-alkyl” refers to a C₁₋₅-acylamino group, as defined herein, attached to a C₂₋₄-alkyl group as defined above. Exemplary C₁₋₅-acylamino-C₂₋₄-alkyl groups include 2-formylaminoethyl and 2-acetyl-aminoethyl. Further, said C₁₋₅-acylamino-C₂₋₄-alkyl groups may be optionally N-substituted with C₁₋₃-alkyl, preferably methyl.

Unless otherwise stated or indicated, the term “heteroaryl” refers to a mono- or bicyclic hydrocarbon ring system comprising at least one aromatic ring and having from 5 to 10 ring atoms and which ring system contains at least one heteroatom such as O, N or S. Said heteroaryl moiety can be linked to the remainder of the molecule via a carbon or nitrogen (provided that the resulting nitrogen is not quaternary) atom in any ring. Examples of heteroaryl groups include furyl, pyrrolyl, thienyl, oxazolyl, isoxazolyl, imidazolyl, thiazolyl, isothiazolyl, pyridinyl, pyrimidinyl, pyrazinyl, chromanyl, quinazolinyl, indolyl, isoindolyl, indolinyl, isoindolinyl, indazolyl, pyrazolyl, pyridazinyl, quinolinyl, isoquinolinyl, benzofuranyl, dihydrobenzofuranyl, benzodioxolyl, benzodioxinyl, benzothienyl, benzimidazolyl, benzothiazolyl, benzothiadiazolyl, and benzotriazolyl groups. The heteroaryl groups may be optionally substituted (e.g., with 1-10 substituents if multicyclic; 1-4 substituents if monocyclic). Optional substituents on heteroaryl are defined elsewhere in the specification and appended claims. If a bicyclic heteroaryl ring is substituted, it may be substituted in any ring.

Unless otherwise stated or indicated, the term “heteroarylcarbonyl” denotes a heteroaryl group that is attached to a carbonyl group, i.e., heteroaryl-(C═O)—.

Unless otherwise stated or indicated, the term “heteroarylcarbonylamino” denotes a heteroarylcarbonyl group that is attached to an amino group, i.e., heteroaryl-(C═O)NH—.

Unless otherwise stated or indicated, the term “heteroarylcarbonylamino-C₂₋₄-alkyl” refers to a heteroarylcarbonylamino group, as defined above, attached to a C₂₋₄-alkyl group as defined above. Exemplary heteroarylcarbonylamino-C₂₋₄-alkyl groups include 2-[(pyridin-3-ylcarbonyl)amino]ethyl, 2-[(pyrazin-2-ylcarbonyl)amino]ethyl, 2-[(1H-pyrrol-2-yl-carbonyl)amino]ethyl, and 2-[(isoxazol-5-ylcarbonyl)amino]ethyl. Further, said heteroaryl-carbonylamino-C₂₋₄-alkyl groups may be optionally N-substituted with C₁₋₃-alkyl, preferably methyl.

Unless otherwise stated or indicated, the term “arylcarbonylamino” refers to an arylcarbonyl group, as defined above, attached to an amino group, i.e., aryl-(C═O)NH—.

Unless otherwise stated or indicated, the term “arylcarbonylamino-C₂₋₄-alkyl” refers to an arylcarbonylamino group, as defined above, attached to a C₂₋₄-alkyl group as defined above. Exemplary arylcarbonylamino-C₂₋₄-alkyl groups include 2-(benzoylamino)ethyl and 3-(benzoylamino)propyl. The aryl portion of said arylcarbonylamino-C₂₋₄-alkyl may be optionally substituted. Optional substituents on said aryl are defined elsewhere in the specification and appended claims. Further, said arylcarbonylamino-C₂₋₄-alkyl groups may be optionally N-substituted with C₁₋₃-alkyl, preferably methyl.

Unless otherwise stated or indicated, the term “heteroarylamino” denotes a heteroaryl group, as defined herein, that is attached to an amino group, i.e., heteroaryl-NH—.

Unless otherwise stated or indicated, the term “heteroarylamino-C₂₋₆-alkyl” refers to a heteroarylamino group, as defined above, attached to a C₂₋₆-alkyl group as defined above. Exemplary heteroarylamino-C₂₋₆-alkyl groups include 2-(pyridin-2-ylamino)ethyl, 2-(pyrazin-2-ylamino)ethyl, 2-(pyridin-3-ylamino)ethyl and 3-(pyridin-2-ylamino)propyl. Further, said heteroarylamino-C₂₋₆-alkyl groups may be optionally N-substituted at the exocyclic nitrogen atom with C₁₋₃-alkyl, preferably methyl.

Unless otherwise stated or indicated, the term “heterocyclyl” refers to a non-aromatic fully saturated or partially unsaturated, preferably fully saturated, monocyclic ring system having 4 to 7 ring atoms with at least one heteroatom such as O, N, or S, and the remaining ring atoms are carbon. Examples of heterocyclic groups include piperidinyl, tetrahydropyranyl, tetrahydrofuranyl, azepinyl, azetidinyl, pyrrolidinyl, morpholinyl, imidazolinyl, thiomorpholinyl, pyranyl, 1,3-dioxolanyl, 1,4-dioxanyl, piperazinyl. When present, the sulfur atom may be in an oxidized form (i.e., S═O or O═S═O). An exemplary heterocyclic group containing sulfur in oxidized form is thiomorpholine 1,1-dioxide. The heterocyclyl groups may be optionally substituted (e.g., with 1-10 substituents if multicyclic; 1-4 substituents if monocyclic). Optional substituents on heteroaryl are defined elsewhere in the specification and appended claims

Unless otherwise stated or indicated, the term “heterocyclylamino” denotes a heterocyclyl group, as defined herein, that is attached to an amino group through a ring carbon of the heterocyclyl group. Exemplary heterocyclylamino groups include piperidin4-ylamino, pyrrolidin-3-ylamino, tetrahydrofuran-2-ylamino and tetrahydropyran-4-ylamino.

Unless otherwise stated or indicated, the term “heterocyclylamino-C₂₋₆-alkyl” refers to a heterocyclylamino group, as defined above, attached to a C₂₋₆-alkyl group as defined above. Exemplary heterocyclylamino-C₂₋₆-alkyl groups include 2-(piperidin-4-yl-amino)ethyl, 3-(pyrrolidin-3-ylamino)propyl, 2-(tetrahydrofuran-2-ylamino)ethyl and 2-(tetrahydropyran-4-ylamino)ethyl. When the heterocyclyl portion of heterocyclylamino-C₂₋₆-alkyl is selected from a nitrogen-containing heterocyclyl group, said heterocyclyl portion may be optionally N-substituted with methyl or ethyl. Exemplary heterocyclyl-amino-C₂₋₆-alkyl groups wherein the heterycycyl portion is optionally N-substituted with methyl or ethyl include 2-(1-methylpiperidin-4-ylamino)ethyl and 3-(1-methylpyrrolidin-3-ylamino)propyl.

Unless otherwise stated or indicated, the term “C₁₋₄-alkylsulfonamido” refers to a group C₁₋₄-alkyl-SO₂NH—.

Unless otherwise stated or indicated, the term “C₁₋₄-alkylsulfonamido-C₂₋₄-alkyl” refers to a C₁₋₄-alkylsulfonamido group, as defined above, attached to a C₂₋₄-alkyl group as defined above. Exemplary C₁₋₄-alkylsulfonamido-C₂₋₄-alkyl groups include 2-(methane-sulfonamido)ethyl and 3-(methanesulfonamido)propyl. Further, said C₁₋₄-alkyl-sulfonamido-C₂₋₄-alkyl groups may be optionally N-substituted with C₁₋₃-alkyl, preferably methyl.

Unless otherwise stated or indicated, the term “C₁₋₄-alkylsulfinamido” refers to a group C₁₋₄-alkyl-SONH—.

Unless otherwise stated or indicated, the term “C₁₋₄-alkylsulfinamido-C₂₋₄-alkyl” refers to a C₁₋₄-alkylsulfinamido group, as defined above, attached to a C₂₋₄-alkyl group as defined above. Exemplary C₁₋₄-alkylsulfinamido-C₂₋₄-alkyl groups include 2-(methane-sulfinamido)ethyl and 3-(methanesulfinamido)propyl. Further, said C₁₋₄-alkylsulfinamido-C₂₋₄-alkyl groups may be optionally N-substituted with C₁₋₃-alkyl, preferably methyl.

Unless otherwise stated or indicated, the term “C₁₋₄-alkylaminosulfonyl” refers to a group C₁₋₄-alkyl-NHSO₂—.

Unless otherwise stated or indicated, the term “C₁₋₄-alkylaminosulfonyl-C₁₋₄-alkyl” refers to a C₁₋₄-alkylaminosulfonyl group, as defined above, attached to a C₁₋₄-alkyl group as defined above. Exemplary C₁₋₄-alkylaminosulfonyl-C₁₋₄-alkyl groups include 2-(methyl-aminosulfonyl)ethyl and 3-(methylaminosulfonyl)propyl. Further, said C₁₋₄-alkylamino-sulfonyl-C₁₋₄-alkyl groups may be optionally N-substituted with C₁₋₃-alkyl, preferably methyl.

Unless otherwise stated or indicated, the term “C₁₋₄-alkylaminosulfinyl” refers to a group C₁₋₄-alkyl-NHSO—.

Unless otherwise stated or indicated, the term “C₁₋₄-alkylaminosulfinyl-C₁₋₄-alkyl” refers to a C₁₋₄-alkylaminosulfinyl group, as defined above, attached to a C₁₋₄-alkyl group as defined above. Exemplary C₁₋₄-alkylaminosulfinyl-C₁₋₄-alkyl groups include 2-(methyl-aminosulfinyl)ethyl and 3-(methylaminosulfinyl)propyl. Further, said C₁₋₄-alkylamino-sulfinyl-C₁₋₄-alkyl groups may be optionally N-substituted with C₁₋₃-alkyl, preferably methyl.

Unless otherwise stated or indicated, the term “C₃₋₆-cycloalkylsulfonamido” refers to a group C₃₋₆-cycloalkyl-SO₂NH—.

Unless otherwise stated or indicated, the term “C₃₋₆-cycloalkylsulfonamido-C₂₋₄-alkyl” refers to a C₃₋₆-cycloalkylsulfonamido group, as defined above, attached to a C₂₋₄-alkyl group as defined above. Exemplary C₃₋₆-cycloalkylsulfonamido-C₂₋₄-alkyl groups include 2-(cyclopropylsulfonamido)ethyl and 3-(cyclopentylsulfonamido)propyl. Further, said C₃₋₆-cycloalkylsulfonamido-C₂₋₄-alkyl groups may be optionally N-substituted with C₁₋₃-alkyl, preferably methyl.

Unless otherwise stated or indicated, the term “C₃₋₆-cycloalkyl-C₁₋₄-alkylsulfonamido” refers to a group C₃₋₆-cycloalkyl-C₁₋₄-alkyl-SO₂NH—.

Unless otherwise stated or indicated, the term “C₃₋₆-cycloalkyl-C₁₋₄-alkylsulfonamido-C₂₋₄-alkyl” refers to a C₃₋₆-cycloalkyl-C₁₋₄-alkylsulfonamido group, as defined above, attached to a C₂₋₄-alkyl group as defined above. Exemplary C₃₋₆-cycloalkyl-C₁₋₄-alkyl-sulfonamido-C₂₋₄-alkyl groups include 2-(cyclopropylmethanesulfonamido)ethyl and 3-[(2-cyclopentylethyl)sulfonamido]propyl. Further, said C₃₋₆-cycloalkyl-C₁₋₄-alkyl-sulfonamido-C₂₋₄-alkyl groups may be optionally N-substituted with C₁₋₃-alkyl, preferably methyl.

Unless otherwise stated or indicated, the term “C₃₋₆-cycloalkylaminosulfonyl” refers to a group C₃₋₆-cycloalkyl-NHSO₂—.

Unless otherwise stated or indicated, the term “C₃₋₆-cycloalkylaminosulfonyl-C₁₋₄-alkyl” refers to a C₃₋₆-cycloalkylaminosulfonyl group, as defined above, attached to a C₁₋₄-alkyl group as defined above. Exemplary C₃₋₆-cycloalkylaminosulfonyl-C₁₋₄-alkyl groups include 2-(cyclopropylaminosulfonyl)ethyl and 3-(cyclopentylaminosulfonyl)propyl. Further, said C₃₋₆-cycloalkylaminosulfonyl-C₁₋₄-alkyl groups may be optionally N-substituted with C₁₋₃-alkyl, preferably methyl.

Unless otherwise stated or indicated, the term “C₃₋₆-cycloalkyl-C₁₋₄-alkyl” refers to a C₃₋₆-cycloalkyl group attached to a C₁₋₄-alkyl group. Exemplary C₃₋₆-cycloalkyl-C₁₋₄-alkyl groups include cyclopropylmethyl, cyclohexylmethyl and 2-cyclohexylethyl.

Unless otherwise stated or indicated, the term “C₃₋₆-cycloalkyl-C₁₋₄-alkylaminosulfonyl” refers to a group C₃₋₆-cycloalkyl-C₁₋₄-alkyl-NHSO₂—.

Unless otherwise stated or indicated, the term “C₃₋₆-cycloalkyl-C₁₋₄-alkylaminosulfonyl-C₁₋₄-alkyl” refers to a C₃₋₆-cycloalkyl-C₁₋₄-alkylaminosulfonyl group, as defined above, attached to a C₁₋₄-alkyl group as defined above. Exemplary C₃₋₆-cycloalkyl-C₁₋₄-alkylaminosulfonyl-C₁₋₄-alkyl groups include 2-(cyclopropylmethylaminosulfonyl)ethyl and 3-[(2-cyclopentylethyl)aminosulfonyl]propyl. Further, said C₃₋₆-cycloalkyl-C₁₋₄-alkyl-aminosulfonyl-C₁₋₄-alkyl groups may be optionally N-substituted with C₁₋₃-alkyl, preferably methyl.

Unless otherwise stated or indicated, the term “C₃₋₆-cycloalkylsulfonyl-C₁₋₄-alkyl” refers to a group C₃₋₆-cycloalkyl-(SO₂)—C₁₋₄-alkyl. Exemplary C₃₋₆-cycloalkylsulfonyl-C₁₋₄-alkyl groups include 2-(cyclopropylsulfonyl)ethyl and 3-(cyclopentylsulfonyl)propyl.

Unless otherwise stated or indicated, the term “C₃₋₆-cycloalkyl-C₁₋₄-alkylsulfonyl-C₁₋₄-alkyl” refers to a group C₃₋₆-cycloalkyl-C₁₋₄-alkyl-(SO₂)—C₁₋₄-alkyl. Exemplary C₃₋₆-cycloalkyl-C₁₋₄-alkylsulfonyl-C₁₋₄-alkyl groups include 2-(cyclopropylmethylsulfonyl)-ethyl and 3-[(2-cyclopentylethyl)sulfonyl]propyl.

Unless otherwise stated or indicated, the term “C₂₋₅-acyl-C₁₋₄-alkyl” refers to a group C₁₋₄-alkyl-(C═O)—C₁₋₄-alkyl. Exemplary “C₂₋₅-acyl-C₁₋₄-alkyl” groups include 2-acetyl-ethyl and 3-acetylpropyl.

Unless otherwise stated or indicated, the term “C₃₋₆-cycloalkylcarbonyl” refers to a C₃₋₆-cycloalkyl group attached to a carbonyl group, i.e., C₃₋₆-cycloalkyl-(C═O)—. Exemplary C₃₋₆-cycloalkylcarbonyl groups include cyclopropylcarbonyl, cyclobutyl-carbonyl, cyclopentylcarbonyl and cyclohexylcarbonyl.

Unless otherwise stated or indicated, the term “C₃₋₆-cycloalkylcarbonyl-C₁₋₄-alkyl” refers to a group C₃₋₆-cycloalkyl-(C═O)—C₁₋₄-alkyl. Exemplary “C₃₋₆-cycloalkylcarbonyl-C₁₋₄-alkyl” groups include 2-(cyclopropylcarbonyl)ethyl and 3-(cyclopentylcarbonyl)propyl.

Unless otherwise stated or indicated, the term “C₃₋₆-cycloalkyl-C₁₋₄-alkylcarbonyl-C₁₋₄-alkyl” refers to a group C₃₋₆-cycloalkyl-C₁₋₄-alkyl-(C═O)—C₁₋₄-alkyl. Exemplary “C₃₋₆-cycloalkyl-C₁₋₄-alkylcarbonyl-C₁₋₄-alkyl” groups include 2-[(2-cyclopropylethyl)-carbonyl]ethyl and 3-(cyclopentylmethylcarbonyl)propyl.

Unless otherwise stated or indicated, the term “C₃₋₆-cycloalkylcarbonylamino” denotes a C₃₋₆-cycloalkylcarbonyl group as defined above attached to an amino group, i.e., C₃₋₆-cycloalkyl-(C═O)NH—.

Unless otherwise stated or indicated, the term “C₃₋₆-cycloalkylcarbonylamino-C₂₋₄-alkyl” refers to a C₃₋₆-cycloalkylcarbonylamino group, as defined above, attached to a C₂₋₄-alkyl group as defined above. Exemplary C₃₋₆-cycloalkylcarbonylamino-C₂₋₄-alkyl groups include 2-(cyclopropylcarbonylamino)ethyl and 2-(cyclobutylcarbonylamino)ethyl.

Unless otherwise stated or indicated, the term “heterocyclyl-C₁₋₆-alkyl” refers to a heterocyclyl group, as defined herein, attached to a C₁₋₆-alkyl group as defined above. Exemplary heterocyclyl-C₁₋₆-alkyl groups include 1,3-dioxolan-2-ylmethyl, 2-(1,3-dioxolan-2-yl)ethyl, tetrahydrofuran-2-ylmethyl, 2-(tetrahydrofuran-2-yl)ethyl and 2-(pyrrolidin-1-yl)ethyl.

Unless otherwise stated or indicated, the term “C₃₋₆-cycloalkyl-C₁₋₄-alkylcarbonylamino” refers to a group “C₃₋₆-cycloalkyl-C₁₋₄-alkyl-(C═O)NH—”.

Unless otherwise stated or indicated, the term “C₃₋₆-cycloalkyl-C₁₋₄-alkylcarbonylamino-C₂₋₄-alkyl” refers to a C₃₋₆-cycloalkyl-C₁₋₄-alkylcarbonylamino group as defined above attached to a C₂₋₄-alkyl group as defined above. Exemplary C₃₋₆-cycloalkyl-C₁₋₄-alkylcarbonylamino-C₂₋₄-alkyl groups include 2-(cyclopropylmethylcarbonylamino)ethyl and 2-[(2-cyclopentylethyl)carbonylamino]ethyl. Further, said C₃₋₆-cycloalkyl-C₁₋₄-alkylcarbonylamino-C₂₋₄-alkyl groups may be optionally N-substituted with C₁₋₃-alkyl, preferably methyl.

Unless otherwise stated or indicated, the term “aminocarbonyl” refers to the radical NH₂(C═O)—.

Unless otherwise stated or indicated, the term “aminocarbonyl-C₁₋₄-alkyl” denotes a C₁₋₄-alkyl group as defined above substituted with an aminocarbonyl group. Exemplary “aminocarbonyl-C₁₋₄-alkyl” groups include 2-(aminocarbonyl)ethyl and 3-(amino-carbonyl)propyl.

Unless otherwise stated or indicated, the term “C₃₋₆-cycloalkylsulfonyl” refers to a group C₃₋₆-cycloalkyl-(SO₂)—.

Unless otherwise stated or indicated, the term “C₃₋₆-cycloalkylsulfinyl” refers to a group C₃₋₆-cycloalkyl-(SO)—.

Unless otherwise stated or indicated, the term “C₁₋₄-alkylaminocarbonyl” refers to a group C₁₋₄-alkyl-NH(C═O)—.

Unless otherwise stated or indicated, the term “C₁₋₄-alkylaminocarbonyl-C₁₋₄-alkyl” refers to a C₁₋₄-alkylaminocarbonyl group, as defined above, attached to a C₁₋₄-alkyl group as defined above. Exemplary “C₁₋₄-alkylaminocarbonyl-C₁₋₄-alkyl” groups include 2-(methyl-aminocarbonyl)ethyl and 3-(ethylaminocarbonyl)propyl. Further, said C₁₋₄-alkylamino-carbonyl-C₁₋₄-alkyl groups may be optionally N-substituted with C₁₋₃-alkyl, preferably methyl.

Unless otherwise stated or indicated, the term “hydroxy-C₁₋₄-alkylaminocarbonyl” refers to a group HO—C₁₋₄-alkyl-NH(C═O)—.

Unless otherwise stated or indicated, the term “hydroxy-C₁₋₄-alkylaminocarbonyl-C₁₋₄-alkyl” refers to a hydroxy-C₁₋₄-alkylaminocarbonyl group, as defined above, attached to a C₁₋₄-alkyl group as defined above. Exemplary “hydroxy-C₁₋₄-alkylaminocarbonyl-C₁₋₄-alkyl” groups include 2-[(2-hydroxyethyl)aminocarbonyl]ethyl and 3-[(2-hydroxyethyl)-aminocarbonyl]propyl. Further, said hydroxy-C₁₋₄-alkylaminocarbonyl-C₁₋₄-alkyl groups may be optionally N-substituted with C₁₋₃-alkyl, preferably methyl.

The term “di-(C₁₋₂-alkyl)amino” refers to a group (C₁₋₂-alkyl)₂N- wherein the two alkyl portions may be the same or different. Exemplary di-(C₁₋₂-alkyl)amino groups include N,N-dimethylamino, N-ethyl-N-methylamino and N,N-diethylamino.

Unless otherwise stated or indicated, the term “di-(C₁₋₂-alkyl)aminocarbonyl” refers to a group (C_{1 -2}-alkyl)₂N(C═O)— wherein the two alkyl portions may be the same or different.

Unless otherwise stated or indicated, the term “di-(C₁₋₂-alkyl)aminocarbonyl-C₁₋₄-alkyl” refers to a di-(C₁₋₂-alkyl)aminocarbonyl group, as defined above, attached to a C₁₋₄-alkyl group as defined above substituted. Exemplary “di-(C₁₋₂-alkyl)aminocarbonyl-C₁₋₄-alkyl” groups include 2-(dimethylaminocarbonyl)ethyl and 3-(diethylaminocarbonyl)propyl.

Unless otherwise stated or indicated, the term “C₃₋₆-cycloalkylaminocarbonyl” refers to a group C₃₋₆-cycloalkyl-NH(C═O)—.

Unless otherwise stated or indicated, the term “C₃₋₆-cycloalkylaminocarbonyl-C₁₋₄-alkyl” refers to a C₃₋₆-cycloalkylaminocarbonyl group, as defined above, attached to a C₁₋₄-alkyl group as defined above. Exemplary “C₃₋₆-cycloalkylaminocarbonyl-C₁₋₄-alkyl” groups include 2-(cyclopropylaminocarbonyl)ethyl and 3-(cyclopentylaminocarbonyl)propyl. Further, said C₃₋₆-cycloalkylaminocarbonyl-C₁₋₄-alkyl groups may be optionally N-substituted with C₁₋₃-alkyl, preferably methyl.

Unless otherwise stated or indicated, the term “C₃₋₆-cycloalkyl-C₁₋₄-alkylaminocarbonyl” refers to a group C₃₋₆-cycloalkyl-C₁₋₄-alkyl-NH(C═O)—.

Unless otherwise stated or indicated, the term “C₃₋₆-cycloalkyl-C₁₋₄-alkylaminocarbonyl-C₁₋₄-alkyl” refers to a C₃₋₆-cycloalkyl-C₁₋₄-alkylaminocarbonyl group, as defined above, attached to a C₁₋₄-alkyl group as defined above. Exemplary “C₃₋₆-cycloalkyl-C₁₋₄-alkyl-aminocarbonyl-C₁₋₄-alkyl” groups include 2-(cyclopropylmethylaminocarbonyl)ethyl and 3-[(2-cyclopentylethyl)aminocarbonyl]propyl. Further, said C₃₋₆-cycloalkyl-C₁₋₄-alkyl-aminocarbonyl-C₁₋₄-alkyl groups may be optionally N-substituted with C₁₋₃-alkyl, preferably methyl.

Unless otherwise stated or indicated, the term “aminocarbonyl-C₁₋₄-alkoxy” refers to a C₁₋₄-alkoxy group as defined above wherein the alkyl portion is substituted with an aminocarbonyl group.

Unless otherwise stated or indicated, the term “aminocarbonyl-C₁₋₄-alkoxy-C₂₋₄-alkyl” refers to an aminocarbonyl-C₁₋₄-alkoxy group, as defined above, attached to a C₂₋₄-alkyl group as defined above. Exemplary aminocarbonyl-C₁₋₄-alkoxy-C₂₋₄-alkyl groups include 2-(2-aminocarbonylethoxy)ethyl and 3-(2-aminocarbonylethoxy)propyl.

Unless otherwise stated or indicated, the term “di-(C₁₋₂-alkyl)aminocarbonyl-C₁₋₄-alkoxy” denotes a C₁₋₄-alkoxy group as defined above wherein the alkyl portion is substituted with a di-(C₁₋₂-alkyl)aminocarbonyl group as defined above.

Unless otherwise stated or indicated, the term “di-(C₁₋₂-alkyl)aminocarbonyl-C₁₋₄-alkoxy-C₂₋₄-alkyl” refers to a di-(C₁₋₂-alkyl)aminocarbonyl-C₁₋₄-alkoxy group, as defined above, attached to a C₂₋₄-alkyl group as defined above. Exemplary di-(C₁₋₂-alkyl)aminocarbonyl-C₁₋₄-alkoxy-C₂₋₄-alkyl groups include 2-[2-(N,N-dimethylaminocarbonyl)ethoxy]ethyl and 3-[2-(N,N-dimethylaminocarbonyl)ethoxy]propyl.

Unless otherwise stated or indicated, the term “C₁₋₄-alkylaminocarbonyl-C₁₋₄-alkoxy” denotes a C₁₋₄-alkoxy group as defined above wherein the alkyl portion is substituted with a C₁₋₄-alkylaminocarbonyl group as defined above.

Unless otherwise stated or indicated, the term “C₁₋₄-alkylaminocarbonyl-C₁₋₄-alkoxy-C₂₋₄-alkyl” refers to a C₁₋₄-alkylaminocarbonyl-C₁₋₄-alkoxy group, as defined above, attached to a C₂₋₄-alkyl group as defined above. Exemplary C₁₋₄-alkylaminocarbonyl-C₁₋₄-alkoxy-C₂₋₄-alkyl groups include 2-[2-(methylaminocarbonyl)ethoxy]ethyl and 3-[2-(methyl-aminocarbonyl)ethoxy]propyl.

Unless otherwise stated or indicated, the term “hydroxy-C₁₋₄-alkylcarbonylamino” refers to a group C₁₋₄-alkyl(C═O)NH— wherein the alkyl portion is substituted with a hydroxy group.

Unless otherwise stated or indicated, the term “hydroxy-C₁₋₄-alkylcarbonylamino-C₂₋₄-alkyl” refers to a hydroxy-C₁₋₄-alkylcarbonylamino group, as defined above, attached to a C₂₋₄-alkyl group as defined above. Exemplary hydroxy-C₁₋₄-alkylcarbonylamino-C₂₋₄-alkyl groups include 2-[(hydroxymethyl)carbonylamino]ethyl and 2-[(2-hydroxyethyl)-carbonylamino]ethyl. Further, said hydroxy-C₁₋₄-alkylcarbonylamino-C₂₋₄-alkyl groups may be optionally N-substituted with C₁₋₃-alkyl, preferably methyl.

Unless otherwise stated or indicated, the term “C₂₋₄-alkynyl” denotes a straight or branched hydrocarbon chain radical containing at least one carbon-carbon triple bond and having from 2 to 4 carbon atoms. Examples of said C₂₋₄-alkynyl groups include ethynyl, 1-propynyl, 2-propynyl, 1-butynyl, 2-butynyl, and 1-methylprop-2-yn-1-yl.

Unless otherwise stated or indicated, the term “C₂₋₄-alkynylcarbonylamino” refers to a group C₂₋₄-alkynyl(C═O)NH—.

Unless otherwise stated or indicated, the term “C₂₋₄-alkynylcarbonylamino-C₂₋₄-alkyl” refers to a C₂₋₄-alkynylcarbonylamino group, as defined above, attached to a C₂₋₄-alkyl group as defined above. Exemplary C₂₋₄-alkynylcarbonylamino-C₂₋₄-alkyl groups include 2-(ethynylcarbonylamino)ethyl and 3-(ethynylcarbonylamino)propyl. Further, said C₂₋₄-alkynylcarbonylamino-C₂₋₄-alkyl groups may be optionally N-substituted with C₁₋₃-alkyl, preferably methyl.

Unless otherwise stated or indicated, the term “hydroxy-C₂₋₄-alkoxy” refers to a C₂₋₄-alkoxy group as defined above in which the alkyl portion is substituted with a hydroxy group.

Unless otherwise stated or indicated, the term “hydroxy-C₂₋₄-alkoxy-C₂₋₄-alkyl” refers to a hydroxy-C₂₋₄-alkoxy group, as defined above, attached to a C₂₋₄-alkyl group as defined above. Exemplary hydroxy-C₂₋₄-alkoxy-C₂₋₄-alkyl groups include 2-(2-hydroxyethoxy)-ethyl, 3-(2-hydroxyethoxy)propyl and 2-(2-hydroxy-2-methylpropoxy)ethyl.

Unless otherwise stated or indicated, the term “C₁₋₄-alkoxy-C₂₋₄-alkoxy-C₂₋₄-alkyl” refers to the group C₁₋₄-alkyl-O—C₂₋₄-alkyl-O—C₂₋₄-alkyl. Exemplary C₁₋₄-alkoxy-C₂₋₄-alkoxy-C₂₋₄-alkyl groups include 2-(2-methoxyethoxy)ethyl and 3-(2-methoxyethoxy)propyl.

Unless otherwise stated or indicated, the term “hydroxy-C₂₋₄-alkoxy-C₂₋₄-alkoxy-C₂₋₄-alkyl” refers to the group HO—(C₂₋₄-alkyl)-O—(C₂₋₄-alkyl)-O—(C₂₋₄-alkyl)-. Exemplary hydroxy-C₂₋₄-alkoxy-C₂₋₄-alkoxy-C₂₋₄-alkyl groups include 2-[2-(2-hydroxyethoxy)-ethoxy]ethyl and 3-[2-(2-hydroxyethoxy)ethoxy]propyl.

Unless otherwise stated or indicated, the term “oxo” denotes ═O (i.e., an oxygen atom joined to a carbon atom through a double bond).

Unless otherwise stated or indicated, the term “C₁₋₅-acylamino” refers to the radical R^{b}(C═O)NH—, wherein R^{b} is selected from hydrogen and C₁₋₄-alkyl.

Unless otherwise stated or indicated, the term “halogen” means fluorine, chlorine, bromine or iodine.

Unless otherwise stated or indicated, the term “hydroxy” refers to the radical —OH.

Unless otherwise stated or indicated, the term “cyano” refers to the radical —CN.

The term “modulate” refers to an increase or decrease in an effect or function. In one aspect, the term “modulate” refers to an increase or decrease, e.g., in the ability of a cell to proliferate in response to exposure to a compound of the invention, e.g., the inhibition of proliferation of at least a sub-population of cells in an animal such that a desired end result is achieved, e.g., a therapeutic result. A “modulator” is a compound that can modulate an effect, function, or response.

The term “metabolic syndrome” refers to a cluster or collection of risk factors that predisposes to cardiovascular disease, including but not restricted to atherosclerosis, coronary artery disease, type 2 diabetes, obesity, hypertension, elevated blood glucose levels or impaired glucose tolerance, high triglycerides and/or LDL levels, hyperlipidemia, hypercholesterolemia, dyslipidemia and hepatic steatosis, including both alcoholic and non-alcoholic steatohepatitis.

“Optional” or “optionally” means that the subsequently described event or circumstance may but need not occur, and that the description includes instances where the event or circumstance occurs and instances in which it does not.

Unless otherwise stated or indicated, the term “attached” is used herein when two chemical groups, as defined above, are joined by a covalent bond.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect).

“Prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2^{nd} Ed., (2004), pp. 498-549, Elsevier Academic Press). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

“Stereoisomer” refers to a compound made up of exactly the same atoms bonded by the same bonds, but having different three-dimensional structures, which are not interchangeable. The present invention includes various stereoisomers and mixtures thereof and includes “enantiomers”, which refers to two stereoisomers which are nonsuperimposable mirror images of one another.

“Tautomer” refers to a shift of a proton from one atom in a molecule to another atom in the same molecule. The present invention includes tautomers of any said compounds.

“Protective groups” include methyl esters, tert-butyl esters, p-nitrobenzyl esters, allyl esters and the like. The protective groups are added to and removed from the intermediate compound according to standard protocols, which are well known to those skilled in the art.

Throughout the specification and the appended claims, a given chemical formula or name shall also encompass all salts, hydrates, solvates, N-oxides and prodrug forms thereof.

Further, a given chemical formula or name shall encompass all tautomeric and stereoisomeric forms thereof. Stereoisomers include enantiomers and diastereomers. Enantiomers can be present in their pure forms, or as racemic (equal) or unequal mixtures of two enantiomers. Diastereomers can be present in their pure forms, or as mixtures of diastereomers. Diastereomers also include geometrical isomers, which can be present in their pure cis or trans forms or as mixtures of those.

The compounds of formula (I) may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

### COMPOSITIONS

For clinical use, the compounds of the invention are formulated into pharmaceutical formulations for various modes of administration. It will be appreciated that compounds of the invention may be administered together with a physiologically acceptable carrier, excipient, or diluent. The pharmaceutical compositions of the invention include those suitable for oral, rectal, nasal, topical (including buccal and sublingual), vaginal, sublingual, intrathecal, transmucosal or parenteral (including subcutaneous, intramuscular, intravenous and intradermal) administration. In certain embodiments, the compounds of the formulae herein are administered transdermally (e.g., using a transdermal patch or iontophoretic techniques). For the treatment of skin diseases, they can also be administered topically. The amount of drug administered will typically be higher when administered orally than when administered, say, intravenously.

Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutical acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the invention may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The compounds of formulae herein may be administered with other active compounds (e.g., therapeutic agents) for the treatment of medical conditions in which the modulation of SCD activity is beneficial, such as cardiovascular diseases, obesity, non-insulin-dependent diabetes mellitus, hypertension, neurological diseases, immune disorders, and cancer; including e.g., type 2 diabetes, coronary artery disease, atherosclerosis, heart disease, cerebrovascular disease, eczema, acne and psoriasis. Such agents are known in the art and include those delineated in the references cited herein, as well as, e.g., insulin and insulin analogs, DPP-IV inhibitors, sulfonyl ureas, biguanides, α2 agonists, glitazones, PPAR-γ agonists, mixed PPAR-α/γ agonists, RXR agonists, α-glucosidase inhibitors, PTP1B inhibitors, 11-β-hydroxy steroid dehydrogenase Type 1 inhibitors, phosphodiesterase inhibitors, glycogen phosphorylase inhibitors, MCH-I antagonists, CB-I antagonists (or inverse agonists), amylin antagonists, CCK receptor agonists, β₃-agonists, leptin and leptin mimetics, serotonergic/dopaminergic antiobesity drugs, gastric lipase inhibitors, pancreatic lipase inhibitors, fatty acid oxidation inhibitors, lipid lowering agents and thyromimetics.

### PREPARATION OF COMPOUNDS OF THE INVENTION

The compounds of formula (I) may be prepared by, or in analogy with, conventional methods. The preparation of intermediates and compounds according to the examples of the present invention may in particular be illuminated by the following Schemes 1-4. Definitions of variables in the structures in schemes herein are commensurate with those of corresponding positions in the formulae delineated herein.

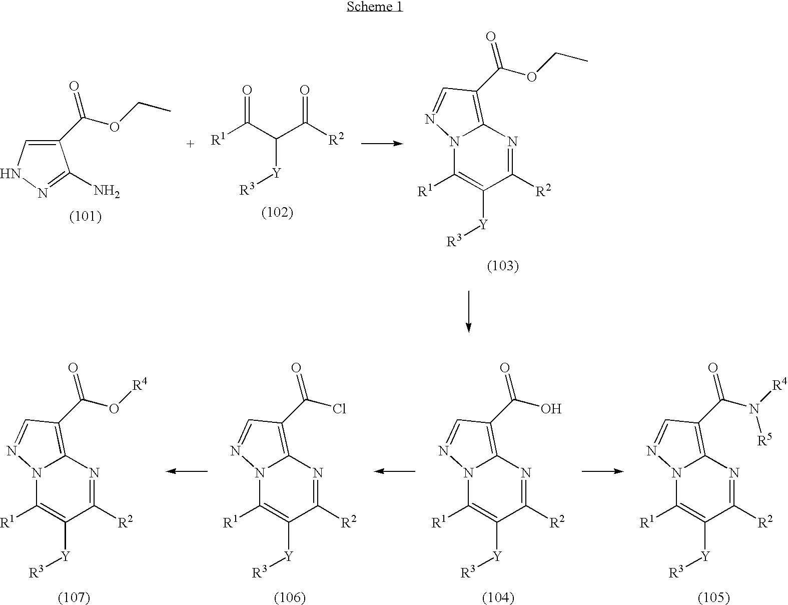

wherein Y═CH₂; and

R¹-R⁵ are as defined in formula (I).

The synthesis of compounds of formula (I), wherein x=0 and W═ —C(O)N(R⁵)— or —C(O)O—, is shown in Scheme 1. Aminopyrazole 101 is reacted with a 1,3-dicarbonyl derivative 102 in the presence of an acid (such as hydrochloric acid) to form the intermediate ester 103, which is subsequently hydrolyzed to the corresponding carboxylic acid 104. Conversion to the corresponding amide 105 can then easily be performed by treating 104 with the appropriate amine in the presence of a suitable coupling reagent (such as 1-propanephosphonic acid cyclic anhydride or TBTU). Alternatively, 104 can be transformed into the corresponding acid chloride 106, which is then treated with the appropriate alcohol to afford ester 107.

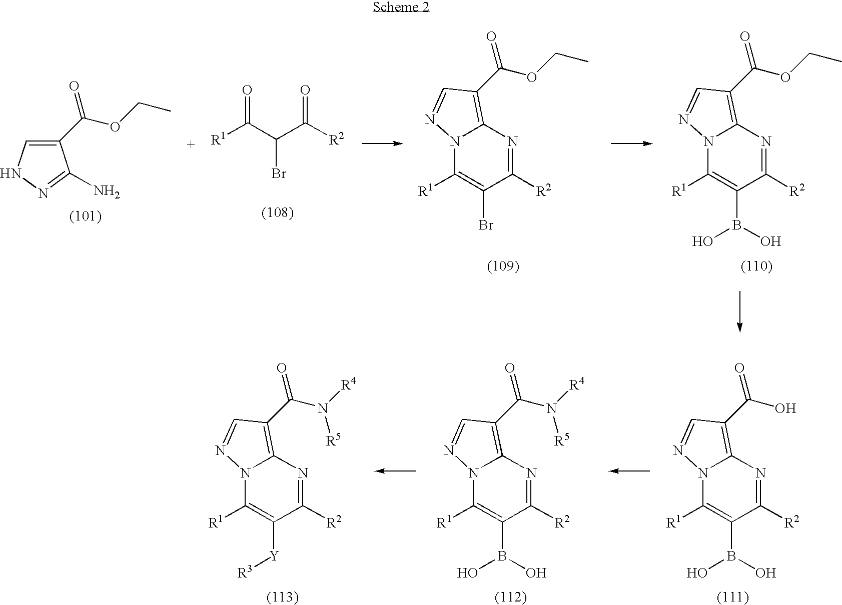

wherein Y═CH₂; and

R¹-R⁵ are as defined in formula (I).

The synthesis of compounds of formula (I), wherein x=0 and W═ —C(O)N(R⁵)—, is depicted in Scheme 2. Condensation of aminopyrazole 101 with a 1,3-dicarbonyl derivative 108 results in the formation of ester 109. Treatment of 109 with bis(pinacolato)diboron transforms the bromide into the corresponding boronic acid 110. Following hydrolysis of the ester group, 111 is then treated with the appropriate amine in the presence of a suitable coupling reagent (such as 1-propanephosphonic acid cyclic anhydride or TBTU) to give the intermediate amide 112. A palladium-catalyzed Suzuki cross-coupling between boronic acid 112 and the appropriate benzylic halide ultimately results in the formation of compound 113.

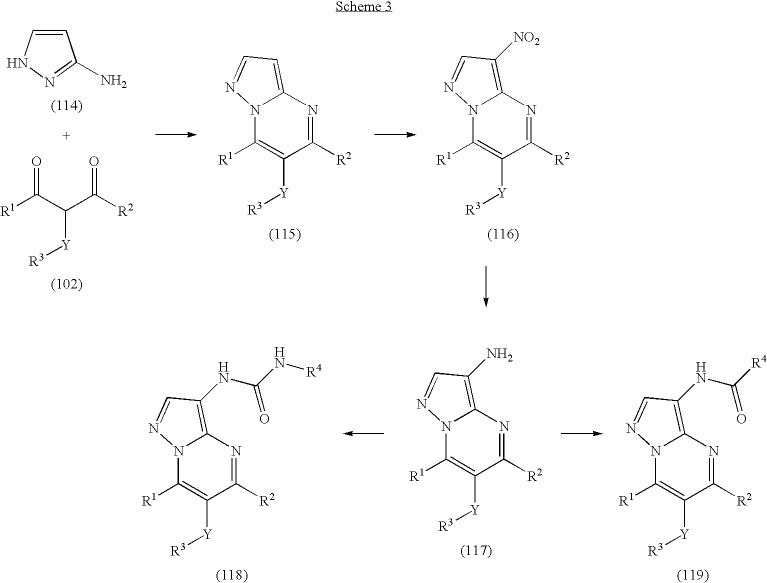

wherein Y═CH₂; and

R¹-R⁴ are as defined in formula (I).

Scheme 3 shows the synthesis of compounds of formula (I), wherein x=0 and W═ —NHC(O)N(H)— or —NHC(O)—. Condensation of aminopyrazole 114 with a 1,3-dicarbonyl derivative 102 results in the formation of the pyrazolo[1,5-α]pyrimidine 115, which is nitrated to give intermediate 116. After reduction of the nitro group, amine 117 is then treated with the appropriate isocyanate to afford the urea compound 118. Alternatively, amine 117 can be treated with the appropriate carboxylic acid in the presence of a suitable coupling agent (such as 1-propanephosphonic acid cyclic anhydride or TBTU) to afford the amide compound 119.

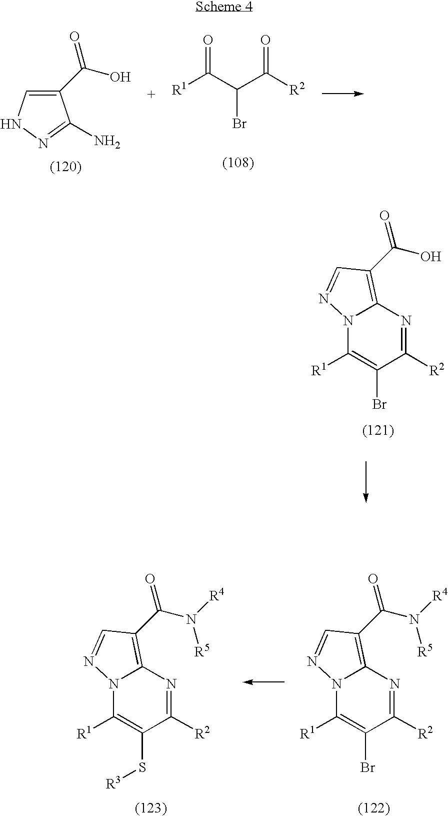

wherein Y═S; and

R¹-R⁵ are as defined in formula (I).

Compounds of formula (I), wherein x=0, Y═S and W═ —C(O)N(R⁵)— can be prepared as shown in Scheme 4. Condensation of aminopyrazole 120 with a 1,3-dicarbonyl derivative 108 results in the formation of carboxylic acid 121, which is treated with the appropriate amine in the presence of a suitable coupling reagent (such as 1-propanephosphonic acid cyclic anhydride or TBTU) to give the intermediate amide 122. A substitution reaction with the appropriate benzenethiol results in the formation of the thio-ether 123.

The necessary starting materials for preparing the compounds of formula (I) are either commercially available, or may be prepared by methods known in the art.

The processes described below in the experimental section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl (triphenylmethyl). The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M.

Fieser, Fieser and Fieser's Reagents for Organic Synthesis, John Wiley and Sons (1994);

L. Paquette, ed., Encyclopedia of Reagents for Organic Synthesis, John Wiley and Sons (1995); and P. J. Kocieński, Protecting Groups, Corrected Edition, Georg Thieme Verlag, Stuttgart (2000), and subsequent editions thereof.

The following abbreviations have been used:
Boc tert-butyloxycarbonyl
CH₃CN acetonitrile
DCM dichloromethane
DMAP 4-(dimethylamino)pyridine
DMF N,N-dimethylformamide
DMSO dimethyl sulfoxide
ESI electrospray ionization
Et₂O diethyl ether
EtOAc ethyl acetate
EtOH ethanol
GC-MS Gas Chromatography Mass Spectroscopy
h hour(s)
HPLC High Performance Liquid Chromatography
HPLC/MS High Performance Liquid Chromatography Mass Spectroscopy
min minute(s)
MS Mass spectroscopy
NMR Nuclear Magnetic Resonance
r.t. room temperature
TBTU O-(benzotriazol-1-yl)-N,N,N′,N′-tetramethyluronium tetrafluoroborate
TFA trifluoroacetic acid
THF tetrahydrofuran

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The invention will now be further illustrated by the following non-limiting Examples. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

### EXAMPLES AND INTERMEDIATE COMPOUNDS

Experimental Methods

¹H nuclear magnetic resonance (NMR) and ¹³C NMR were recorded on a Bruker Advance DPX 400 spectrometer at 400.1 and 100.6 MHz, respectively. All spectra were recorded using residual solvent or tetramethylsilane (TMS) as internal standard. Preparative HPLC/MS was performed on a Waters/Micromass Platform ZQ system and preparative HPLC/UV was performed on a Gilson system in accordance to the experimental details specified in the examples. Analytical HPLC/MS was performed using an Agilent 1100/1200 Series Liquid Chromatograph/Mass Selective Detector (MSD) (Single Quadrupole) (1946A/1946C/1956C/6110) equipped with an electrospray interface. GC-MS was performed on a Hewlett-Packard 5890/6890 gas chromatograph equipped with a HP-5MS crosslinked 5% PhMe Siloxane column (30 m×0.25 mm×0.25 μm film thickness) with a Hewlett-Packard 5971A/5972A mass selective detector using EI. Preparative flash chromatography was performed on Merck silica gel 60 (230-400 mesh). The compounds were named using ACD Name 6.0 or ACD 7.0 or ACD 8.0. Microwave reactions were performed with a Personal Chemistry Smith Creator or Optimizer using 0.5-2 mL or 2-5 mL Smith Process Vials fitted with aluminum caps and septa. Accurate masses are measured using an Agilent MSD-TOF connected to an Agilent 1100 HPLC system. During the analyses the calibration is checked by two masses and automatically corrected when needed. Spectra are acquired in positive electrospray mode. The acquired mass range is m/z 100-1100. Profile detection of the mass peaks is used.

### Intermediate 1

### Dimethyl 2-(3,4-dichlorobenzyl)malonate

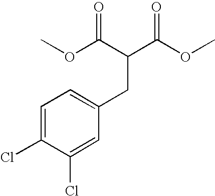

Dimethyl malonate (2.5 g, 19 mmol) was dissolved in dry THF (15 mL) and the solution cooled on an ice-bath. NaH (0.302 g, 7.60 mmol, 60% in mineral oil) was added followed by 1,2-dichloro-4-(chloromethyl)benzene (1.2 g, 6.3 mmol) and it was stirred at r.t. for 30 min. Et₂O (5 mL) and hexane (2 mL) were added to the reaction mixture and the resulting solution was washed with sat NH₄Cl (3x5 mL). The organic phase was evaporated overnight and then dried in a vacuum oven at 60° C. to give the crude title compound (0.70 g, 39%) as a light yellow solid.

### Intermediate 2

### Dimethyl (4-bromobenzyl)malonate

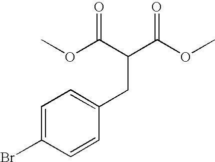

According to the experimental procedure for INTERMEDIATE 1, dimethyl malonate (2.5 g, 19 mmol), NaH (0.302 g, 7.60 mmol, 60% in mineral oil) and 1-bromo-4-(bromo-methyl)benzene (1.6 g, 6.3 mmol) were reacted to give the crude title compound (0.78 g, 41%) as an off-white solid.

### Intermediate 3

### Dimethyl (3-chloro-4-fluorobenzyl)malonate

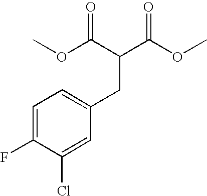

According to the experimental procedure for INTERMEDIATE 1, dimethyl malonate (2.5 g, 19 mmol), NaH (0.302 g, 7.60 mmol, 60% in mineral oil) and 4-(bromomethyl)-2-chloro-1-fluorobenzene (1.6 g, 6.3 mmol) were reacted to give the crude title compound (1.3 g, 75%) as a light yellow solid.

### Intermediate 4

### Dimethyl[4-chloro-3-(trifluoromethoxy)benzyl]malonate

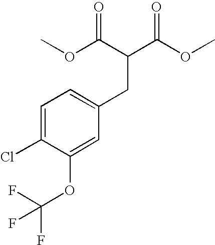

Sodium hydride (264 mg, 6.60 mmol, 60% in mineral oil) was suspended in dry THF (20 mL) and cooled on an ice-bath. Dimethyl malonate (0.79 g, 6.0 mmol) was added dropwise under hydrogen evolution and the reaction mixture left to stir for 30 min. 4-(Bromomethyl)-1-chloro-2-(trifluoromethoxy)benzene (0.82 g, 3.0 mmol) was added and the mixture stirred on the thawing ice-bath overnight. The reaction mixture was worked up by pouring on 1M HCl (100 mL) and diethyl ether (100 mL). Shaking, separating, washing with sat NH₄Cl, drying of the organic phase (Na2SO₄), filtration and evaporation gave the crude product as a clear oil containing excess dimethyl malonate. The oil was put under a gentle nitrogen flow overnight at rt, which removed dimethyl malonate effectively to give the title compound (0.73 g, 61%). The crude product was used in following reaction steps without purification.

### Intermediate 5

### Dimethyl [4-chloro-3-(trifluoromethyl)benzyl]malonate

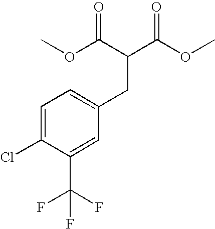

According to the experimental procedure for INTERMEDIATE 4, sodium hydride (264 mg, 6.60 mmol, 60% in mineral oil), dimethyl malonate (0.79 g, 6.0 mmol) and 4-(bromomethyl)-1-chloro-2-(trifluoromethyl)benzene (0.82 g, 3.0 mmol) were reacted to give the crude title compound (1.07 g, 99%).

### Intermediate 6

### Dimethyl[4-fluoro-3-(trifluoromethyl)benzyl]malonate

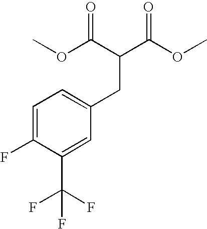

According to the experimental procedure for INTERMEDIATE 1, dimethyl malonate (2.5 g, 19 mmol), NaH (0.302 g, 7.60 mmol, 60% in mineral oil) and 4-(bromomethyl)-1-fluoro-2-(trifluoromethyl)benzene (1.6 g, 6.3 mmol) were reacted to give the crude title compound (0.76 g, 39%).

### Intermediate 7

### Dimethyl {1-[3-(trifluoromethyl)phenyl]ethyl}malonate

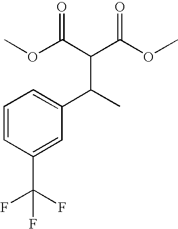

Dimethyl malonate (1.6 g, 12 mmol) was dissolved in dry THF (15 mL) and the solution cooled on an ice-bath. NaH (0.187 g, 4.70 mmol, 60% in mineral oil) was added followed by 1-(1-bromoethyl)-3-(trifluoromethyl)benzene (1.0 g, 3.9 mmol) and it was stirred at r.t. overnight. Et₂O (5 mL) and hexane (2 mL) were added to the reaction mixture and the resulting solution was washed with sat NH₄Cl (3×5 mL). The organic phase was evaporated overnight and then dried in a vacuum oven at 60° C. to give the crude title compound (0.90 g, 76%) as a light yellow gum.

### Intermediate 8

### 6-(3,4-Dichlorobenzyl)pyrazolo[1,5-α]pyrimidine-3-carboxylic acid

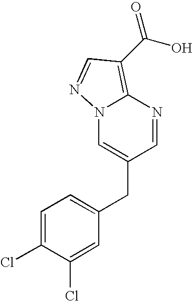

Dimethyl 2-(3,4-dichlorobenzyl)malonate (INTERMEDIATE 1, 1.07 g, 3.29 mmol) was dissolved in dry DCM (20 mL) and the solution was cooled to −78° C. Diisobutyl-aluminium hydride (40 mL, 1M in hexanes) was added to the pre-cooled solution during 3.5 h. After completed addition the reaction was quenched by addition of a solution of ethyl 3-amino-1H-pyrazole-4-carboxylate (3.06 g, 19.7 mmol) in MeOH (10 mL) during 20 min. After completed addition conc. HCl (1.9 mL) was added and the solvents were evaporated. The remaining solids were suspended in EtOH (10 mL) and after pH check more HCl (1 mL) was added to obtain an acidic reaction mixture. The reaction mixture was stirred at r.t. overnight, transferred to a separatory funnel and 1 M HCl (150 mL) was added. The suspension was extracted with Et₂O (200+150 mL), the combined etheral phases dried (MgSO₄) and evaporated. The residual solids (1.58 g) were suspended in EtOH (5 mL), 1M KOH (5 mL) was added and the mixture stirred at 90° C. for 30 min. After cooling to ambient temperature the reaction mixture was washed with toluene. The aqueous phase was acidified followed by extraction with Et₂O (3×50 mL). The combined organic layers were dried (MgSO₄) and concentrated to give the title compound (0.97 g, 73% purity, 67%). The product was used without further purification.

### Intermediate 9

### 6-(4-Bromobenzyl)pyrazolo[1,5-α]pyrimidine-3-carboxylic acid

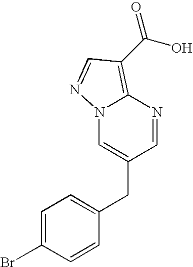

Crude dimethyl (4-bromophenyl)malonate (INTERMEDIATE 2, 0.78 g, 2.6 mmol) was dissolved in dry DCM (7 mL) and the solution cooled to −78° C. Diisobutylaluminium hydride (7 mL, 1M in hexanes) was added to the pre-cooled solution during 2 h. After completed addition ethyl 3-amino-1H-pyrazole-4-carboxylate (2.6 mmol, 5.1 mL of a 0.50M solution in EtOH) was added dropwise. A gentle stream of N₂ was applied and the reaction mixture was heated at 40° C. to evaporate the solvents. After completed evaporation EtOH (7 mL) and concentrated HCl (0.5 mL) were added and the reaction mixture was heated at 110° C. overnight. After 17 h additional concentrated HCl (440 μL) was added and heating was continued at 110° C. After additional 6 h the reaction was complete and 1M KOH (7 mL) was added and the reaction stirred at 90° C. overnight. More 1M KOH was added after 20 h to adjust the pH to 9 and heating was continued. After 5 h the pH was further adjusted to 14 and the reaction stirred at 90° C. overnight. The reaction mixture was cooled and acidified and the suspension was subjected to centrifugation to isolate the solid product. The solids were washed with toluene and water and centrifuged after each washing. Remaining water was co-evaporated with toluene and the obtained solid dried over night in a vacuum oven to give the title compound (0.83 g, 73% purity, 73%) as an off-white powder. ¹H NMR (400 MHz, DMSO-d₆) δ ppm 4.05-4.07 (m, 1 H) 7.30-7.33 (m, 1 H) 7.48-7.52 (m, 1 H) 8.52-8.53 (m, 1 H) 8.72 (d, 1 H) 9.21 (d, 1 H).

### Intermediate 10

### 6-(3-Chloro-4-fluorobenzyl)pyrazolo[1,5-α]pyrimidine-3-carboxylic acid

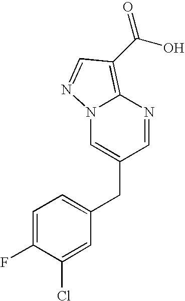

Dimethyl 2-(3-chloro-4-fluorobenzyl) malonate (INTERMEDIATE 3, 1.8 g, 6.6 mmol) was dissolved in dry DCM (20 mL) and the solution was cooled to −78° C. Diisobutylaluminium hydride (20 mL, 1M in hexanes) was added to the pre-cooled solution during 2 h. After completed addition ethyl 3-amino-1H-pyrazole-4-carboxylate (14 mL, 0.50M in EtOH) was added dropwise. A gentle stream of N₂ was applied and the reaction mixture was heated at 40° C. to evaporate the solvents. After completed evaporation EtOH (20 mL) and concentrated HCl (3 mL) were added and the reaction mixture was stirred overnight at room temperature. 1M KOH (30 mL) was added and the reaction stirred at 90° C. overnight. More 1M KOH was added after 20 h to adjust the pH to 9 and heating was continued. After 5 h the pH was further adjusted to 14 and the reaction stirred at 90° C. overnight. The reaction was cooled and acidified and the suspension was subjected to centrifugation to isolate the solid product. The solids were washed with toluene and water and centrifuged after each washing. Remaining water was co-evaporated with toluene and the obtained solid dried overnight in a vacuum oven to give the title compound (2.82 g, 65% purity) as an off-white powder.

### Intermediate 11

### 6-[4-Chloro-3-(trifluoromethoxy)benzyl]pyrazolo[1,5-α]pyrimidine-3-carboxylic acid

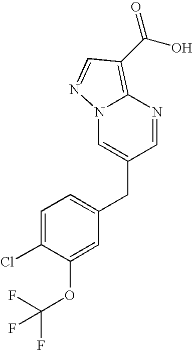

Crude dimethyl[4-chloro-3-(trifluoromethoxy)benzyl]malonate (INTERMEDIATE 4, 0.73 g, 2.1 mmol) was dissolved in dry DCM (5 mL) and the solution cooled to −78° C. Diisobutylaluminium hydride (5 mL, 1M in hexanes) was added to the pre-cooled solution during 1 h. 30 min after completed addition ethyl 3-amino-1H-pyrazole-4-carboxylate (0.33 g, 2.1 mmol) in MeOH (4 mL) was added dropwise during 20 min. After completed addition concentrated HCl (0.2 mL) was added and the solvents were evaporated. The remaining solids were suspended in EtOH (6 mL) and after pH check another amount of HCl (0.2 mL) was added to obtain an acidic reaction mixture. The reaction mixture was heated at 110° C. for 2 days, transferred to a separatory funnel and 1M HCl was added. The suspension was extracted with Et₂O (2×150+50 mL), the combined etheral phases dried (MgSO₄) and evaporated. The residual solids were suspended in EtOH (3 mL), 1M KOH (3 mL) was added and the mixture stirred at 90° C. for 30 min. After cooling to ambient temperature the reaction mixture was washed with toluene. The aqueous phase was acidified followed by extraction with Et₂O (3×25 mL). The combined organic layers were dried (MgSO₄) and concentrated to give the title compound (0.44 g, 60% purity). The product was used without further purification.

### Intermediate 12

### 6-14-Chloro-3-(trifluoromethyl)benzyl]pyrazolo[1,5-α]pyrimidine-3-carboxylic acid

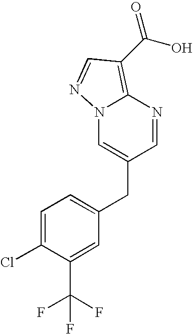

Dimethyl[4-chloro-3-(trifluoromethyl)benzyl]malonate (INTERMEDIATE 5, 0.96 g, 3.0 mmol) was dissolved in dry DCM (5 mL) and the solution cooled to −78° C. Diisobutylaluminium hydride (5 mL, 1M in hexanes) was added to the pre-cooled solution during 1 h. 30 min after completed addition ethyl 3-amino-1H-pyrazole-4-carboxylate (0.33 g, 2.1 mmol) in MeOH (4 mL) was added dropwise during 20 min. After completed addition concentrated HCl (0.25 mL) was added and the solvents were evaporated. The remaining solids were suspended in EtOH (6 mL) and after pH check another amount of HCl (0.25 mL) was added to obtain an acidic reaction mixture. The reaction mixture was heated at 110° C. for 2 days, transferred to a separatory funnel and 1M HCl was added. The suspension was extracted with Et₂O (180+60 mL), the combined etheral phases dried (MgSO₄) and evaporated. The residual solids were suspended in EtOH (3 mL), 1M KOH (3 mL) was added and the mixture stirred at 90° C. for 30 min. After cooling to ambient temperature the reaction mixture was washed with toluene. The aqueous phase was acidified followed by extraction with Et₂O (3×25 mL). The combined organic layers were dried (MgSO₄) and concentrated to give the title compound (0.86 g, 66% purity). The product was used without further purification.

### Intermediate 13

### Ethyl 6-{1-[3-(trifluoromethyl)phenyl]ethyl}pyrazolo[1,5-α]pyrimidine-3-carboxylate

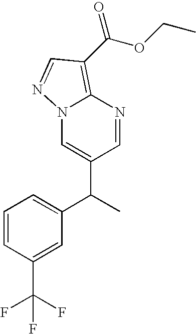

Dimethyl {1-[3-(trifluoromethyl)phenyl]ethyl}malonate (INTERMEDIATE 7, 0.90 g, 3.0 mmol) was dissolved in dry Et₂O (6 mL) and the solution cooled to −78° C. Diisobutylaluminium hydride (10 mL, 1M in hexanes) was added to the pre-cooled solution during 2 h. The reaction was quenched with MeOH and left to warm to 0° C. A saturated aqueous solution of Rochelle salt was added and the mixture diluted with Et₂O and MeOH. The formed precipitate was filtered off and the filtrate evaporated. The thus obtained crude dialdehyde (0.10 g, 0.40 mmol) was suspended in EtOH (7 mL), treated with ethyl 3-amino-1H-pyrazole-4-carboxylate (64 mg, 0.40 mmol) and concentrated hydrochloric acid (30 μL) and the reaction stirred at 50° C. for one hour. The solvents were removed in vacuo, the residue diluted with 1M HCL (5 mL) and extracted with Et₂O (2×25 mL). The combined etheral phases were filtered through a pad of MgSO₄ and evaporated to give the crude title compound (27 mg).

### Intermediate 14

### 6-{1-[3-(Trifluoromethyl)phenyl]ethyl}pyrazolo[1,5-α]pyrimidine-3-carboxylic acid

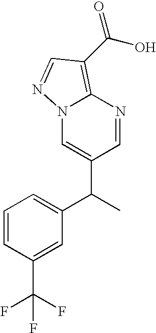

A solution of crude ethyl 6-{1-[3-(trifluoromethyl)phenyl]ethyl}pyrazolo[1,5-α]-pyrimidine-3-carboxylate (INTERMEDIATE 13, 27 mg, 0.074 mmol) in EtOH (2 mL) was treated with 1M KOH (0.2 mL) and stirred at r.t. for 50 min. The reaction mixture was washed with toluene, the aqueous phase acidified with 1M HCl and extracted with Et₂O. The combined etheral phases were filtrated through a pad of MgSO₄ and evaporated to give the title compound (13.6 mg, 90% purity).

### Example 1

### tert-Butyl[2-({[6-(3,4-dichlorobenzyl)pyrazolo[1,5-α]pyrimidin-3-yl]carbonyl}-amino)ethyl]carbamate

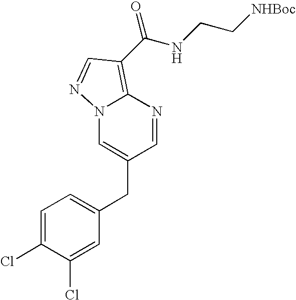

A solution of 6-(3,4-dichlorobenzyl)pyrazolo[1,5-α]pyrimidine-3-carboxylic acid (INTERMEDIATE 8, 45 mg, 0.15 mmol) in DMF (6 mL) was treated with tert-butyl (2-aminoethyl)carbamate (27 mg, 0.17 mmol) followed by TBTU (57 mg, 0.18 mmol) and triethylamine (18 mg, 0.18 mmol). The mixture was stirred at r.t. overnight and purified by preparative HPLC (XTerra C18, 50 mM NH₄HCO₃ pH 10—CH₃CN) to give the title compound as a brown solid (60 mg, 86%). A part of the product (5 mg) was repurified by preparative HPLC (ACE C_{8, 0.1}% TFA—CH₃CN) to give the title compound as a white solid (0.2 mg). MS (ESI+) calcd for C₂₁H₂₃Cl₂N₅O₃ 463.1178, found 463.1177.

### Example 2

### 6-(3,4-dichlorobenzyl)-N-{2-[(pyrazin-2-ylcarbonyl)amino]ethyl}pyrazolo[1,5-α]-pyrimidine-3-carboxamide

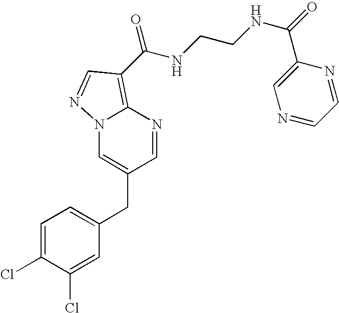

tert-Butyl[2-({[6-(3,4-dichlorobenzyl)pyrazolo[1,5-α]pyrimidin-3-yl]carbonyl}amino)-ethyl]carbamate (EXAMPLE 1, 60 mg, 0.13 mmol)) was dissolved in a mixture of DCM/TFA (4 mL, 50:50) and stirred at r.t. for 30 min. The reaction mixture was concentrated to give 2-({[6-(3,4-dichlorobenzyl)pyrazolo[1,5-α]pyrimidin-3-yl]carbonyl}-amino)ethanaminium trifluoroacetate (45 mg, 95%) as a yellow gum.

The crude trifluoroacetate (15 mg, 0.040 mmol) was dissolved in DMF (2 mL) and treated with 2-pyrazinecarboxylic acid (6.5 mg, 0.050 mmol) followed by TBTU (17 mg, 0.054 mmol) and triethylamine (5.4 mg, 0.054 mmol). The mixture was stirred at r.t. for 3 h and then purified using preparative HPLC (ACE C_{8, 0.1}% TFA—CH₃CN) to give the title compound (0.9 mg, 5%) as a white solid. MS (ESI+) calcd for C₂₁H₁₇Cl₂N₇O₂ 469.0821, found 469.0821.

### Intermediate 15

### 6-(3,4-dichlorobenzyl)-N-[2-(methylthio)ethyl]pyrazolo[1,5-α]pyrimidine-3-carboxamide

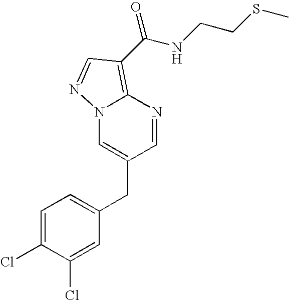

A solution of 6-(3,4-dichlorobenzyl)pyrazolo[1,5-α]pyrimidine-3-carboxylic acid (INTERMEDIATE 8, 30 mg, 0.090 mmol) in DMF (1.5 mL) was treated with TBTU (15 mg, 0.050 mmol) and triethylamine (6 μL, 0.05 mmol) followed by 2-(methylsulfanyl)ethanamine (10.2 mg, 0.110 mmol). The mixture was stirred at r.t. overnight and purified by preparative HPLC (XTerra C18, 50 mM NH₄HCO₃ pH 10—CH₃CN) to give the title compound (12 mg, 34%).

### Example 3

### 6-(3,4-Dichlorobenzyl)-N-[2-(methylsulfinyl)ethyl]pyrazolo[1,5-α]pyrimidine-3-carboxamide

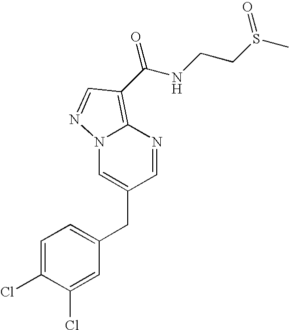

6-(3,4-Dichlorobenzyl)-N-[2-(methylthio)ethyl]pyrazolo[1,5-α]pyrimidine-3-carboxamide (INTERMEDIATE 15, 4 mg, 0.01 mmol) was dissolved in phenol (0.5 mL) and treated with 30% (w/w) hydrogen peroxide (0.6 μL, 0.02 mmol) at r.t. for 1 min. The reaction mixture was purified by preparative HPLC (XTerra C18, 50 mM NH4HCO₃ pH 10—CH₃CN) to give the title compound (1 mg) as a white solid. MS (ESI+) calcd for C₁₇H₁₆Cl₂N₄O₂S 410.0371, found 410.0366.

### Example 4

### 6-(3,4-Dichlorobenzyl)-N-[2-(methylsulfonyl)ethyl]pyrazolo[1,5-α]pyrimidine-3-carboxamide

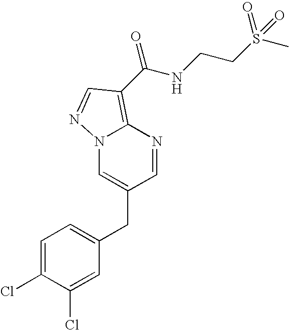

6-(3,4-Dichlorobenzyl)-N-[2-(methylthio)ethyl]pyrazolo[1,5-α]pyrimidine-3-carboxamide (INTERMEDIATE 15, 4 mg, 0.01 mmol) was dissolved in DCM (0.5 mL) and treated with mCPBA (15 mg, 0.090 mmol) in portions and stirred at r.t. for 5 days. More mCPBA was added and after one additional day the reaction was purified by preparative HPLC (XTerra C18, 50 mM NH₄HCO₃ pH 10—CH₃CN) to give the title compound (0.6 mg). MS (ESI+) calcd for C₁₇H₁₆Cl₂N₄O₃S 426.032, found 426.0314.

### Example 5

General Procedure A

### 6-(3,4-Dichlorobenzyl)-N-[2-(dimethylamino)-2-oxoethyl]pyrazolo[1,5-α]pyrimidine-3-carboxamide

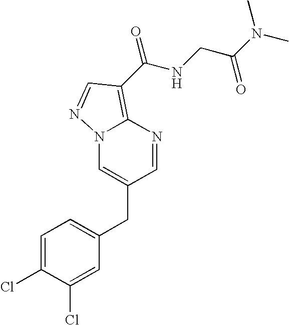

A solution of 6-(3,4-dichlorobenzyl)pyrazolo[1,5-α]pyrimidine-3-carboxylic acid (INTERMEDIATE 8, 10 mg, 0.030 mmol) in DMF (1 mL) was treated with TBTU (15 mg, 0.050 mmol), triethylamine (6 μL, 0.05 mmol) and 2-(dimethylamino)-2-oxoethanaminium acetate (6.0 mg, 0.038 mmol). The mixture was stirred at r.t. overnight. The crude product was purified by preparative HPLC (XTerra C18, 50 mM NH₄HCO₃ pH 10—CH₃CN). MS (ESI+) calcd for C₁₈H₁₇Cl₂N₅O₂ 405.0759, found 405.0750.

### Example 6

### 6-(3,4-Dichlorobenzyl)-N-[2-(methylamino)-2-oxoethyl]pyrazolo[1,5-α]pyrimidine-3-carboxamide

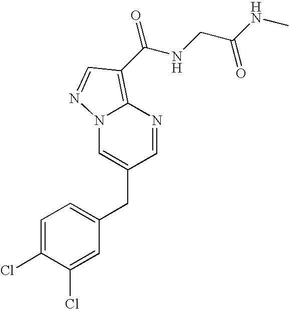

The title product was prepared according to General procedure A, using 2-amino-N-methylacetamide (4.6 mg, 0.038 mmol) as the amine. The crude product was purified by preparative HPLC (XTerra C18, 50 mM NH₄HCO₃ pH 10—CH₃CN). MS (ESI+) calcd for C₁₇H₁₅Cl₂N₅O₂ 391.0603, found 391.0606.

### Example 7

### N-[2-(Benzyloxy)ethyl]-6-(3,4-dichlorobenzyl)pyrazolo[1,5-α]pyrimidine-3-carboxamide

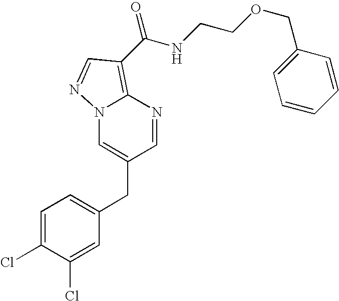

The title product was prepared according to General procedure A, using 2-(benzyloxy)-ethanamine (5.6 mg, 0.038 mmol) as the amine. The crude product was purified by preparative HPLC (XTerra C18, 50 mM NH₄HCO₃ pH 10—CH₃CN). MS (ESI+) calcd for C₂₃H₂₀Cl₂N₄O₂ 454.0963, found 454.0954.

### Example 8

### 6-(3,4-Dichlorobenzyl)-N-(3-methoxypropyl)pyrazolo[1,5-α]pyrimidine-3-carboxamide

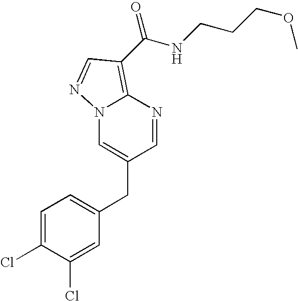

The title product was prepared according to General procedure A, using 3-methoxypropan-1-amine (3.3 mg, 0.038 mmol) as the amine. The crude product was purified by preparative HPLC (XTerra C18, 50 mM NH₄HCO₃ pH 10—CH₃CN). MS (ESI+) calcd for C₁₈H₁₈Cl₂N₄O₂ 392.0807, found 392.0798.

### Example 9

### 6-(3,4-Dichlorobenzyl)-N-(3-hydroxypropyl)pyrazolo[1,5-α]pyrimidine-3-carboxamide

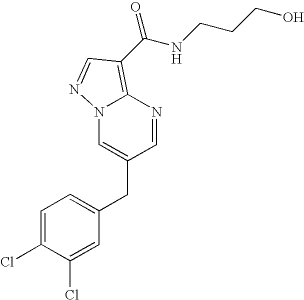

The title product was prepared according to General procedure A, using 3-aminopropan-1-ol (2.8 mg, 0.038 mmol) as the amine. The crude product was purified by preparative HPLC (XTerra C18, 50 mM NH₄HCO₃ pH 10—CH₃CN). MS (ESI+) calcd for C₁₇H₁₆Cl₂N₄O₂ 378.065, found 378.0649.

### Example 10

### 6-(3,4-Dichlorobenzyl)-N-(tetrahydrofuran-2-ylmethyl)pyrazolo[1,5-α]pyrimidine-3-carboxamide

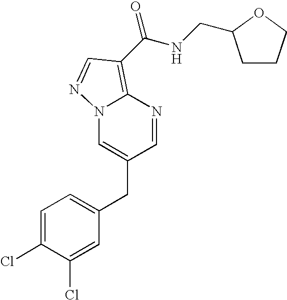

The title product was prepared according to General procedure A, using 1-(tetrahydro-furan-2-yl)methanamine (3.8 mg, 0.038 mmol) as the amine. The crude product was purified by preparative HPLC (XTerra C18, 50 mM NH₄HCO₃ pH 10—CH₃CN). MS (ESI+) calcd for C₁₉H₁₈Cl₂N₄O₂ 404.0807, found 404.0800.

### Example 11

General Procedure B

### 6-(3,4-Dichlorobenzyl)-N-[2-(isonicotinoylamino)ethyl]pyrazolo[1,5-α]pyrimidine-3-carboxamide

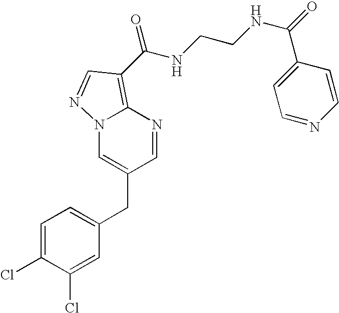

A solution of 6-(3,4-dichlorobenzyl)pyrazolo[1,5-α]pyrimidine-3-carboxylic acid (INTERMEDIATE 8, 15 mg, 0.047 mmol) in DMF (1 mL) was treated with a solution of TBTU (23 mg, 0.075 mmol) and triethylamine (10 μL, 0.075 mmol) in DMF (1 mL) followed by N-(2-aminoethyl)pyridine-4-carboxamide (9.3 mg, 0.056 mmol). The mixture was stirred at r.t. for 45 min. The crude product was purified by preparative HPLC (XTerra C18, 50 mM NH₄HCO₃ pH 10—CH₃CN). MS (ESI+) calcd for C₂₂H₁₈Cl₂N₆O₂ 468.0868, found 468.0872.

### Example 12

### 6-(3,4-Dichlorobenzyl)-N-12-(pyridin-2-ylamino)ethyl]pyrazolo[1,5-α]pyrimidine-3-carboxamide

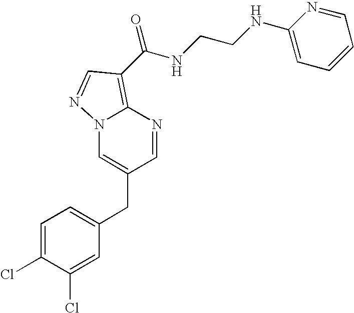

The title product was prepared according to General procedure B, using N-pyridin-2-yl-ethane-1,2-diamine (7.7 mg, 0.056 mmol) as the amine. The crude product was purified by preparative HPLC (XTerra C18, 50 mM NH₄HCO₃ pH 10—CH₃CN). MS (ESI+) calcd for C₂₁H₁₈Cl₂N₆O 440.0919, found 440.0932.

### Example 13

### 6-(3,4-Dichlorobenzyl)-N-12-(2-furyl)ethyl]pyrazolo[1,5-α]pyrimidine-3-carboxamide

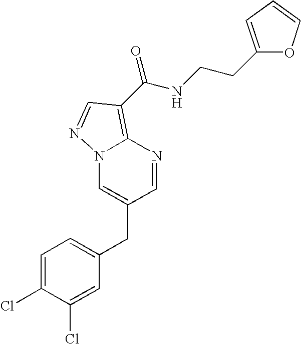

The title product was prepared according to General procedure B, using 2-furan-2-yl-ethanamine (6.2 mg, 0.056 mmol) as the amine. The crude product was purified by preparative HPLC (XTerra C18, 50 mM NH₄HCO₃ pH 10—CH₃CN). MS (ESI+) calcd for C₂₀H₁₆Cl₂N₄O₂ 414.0650, found 414.0658.

### Example 14

### 6-(3,4-Dichlorobenzyl)-N-{2-[(2-furylmethyl)thio]ethyl}pyrazolo[1,5-α]pyrimidine-3-carboxamide

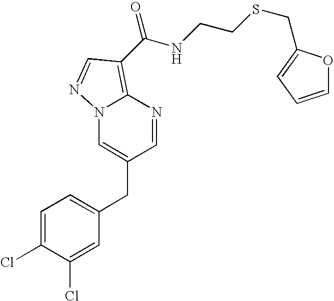

The title product was prepared according to General procedure B, using 2-[(furan-2-yl-methyl)sulfanyl]ethanamine (8.8 mg, 0.056 mmol) as the amine. The crude product was purified by preparative HPLC (XTerra C18, 50 mM NH₄HCO₃ pH 10—CH₃CN). MS (ESI+) calcd for C₂₁H₁₈Cl₂N₄O₂S 460.0528, found 460.0544.

### Example 15

### N-(3-Amino-3-oxopropyl)-6-(3,4-dichlorobenzyl)pyrazolo[1,5-α]pyrimidine-3-carboxamide

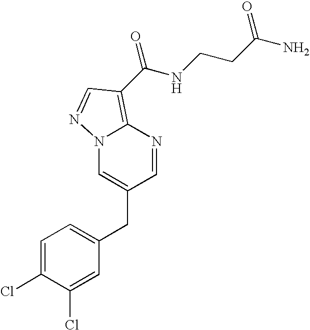

A solution of β-alanine hydrochloride (10 mg, 0.11 mmol) in DMF (0.5 mL) was added to solid 6-(3,4-dichlorobenzyl)pyrazolo[1,5-α]pyrimidine-3-carboxylic acid (INTERMEDIATE 8, 32.2 mg, 0.100 mmol) in a tube and the suspension was stirred at r.t. Triethylamine (21 μL, 0.15 mmol) and a solution of TBTU (48 mg, 0.15 mmol) in DMF (1 mL) were added to the suspension and stirring continued for 30 min at r.t. The reaction mixture was concentrated and purified by preparative HPLC (XTerra C18, 50 mM NH₄HCO₃ pH 10—CH₃CN) to give the title compound (1 mg, 3%) as a white solid. MS (ESI+) calcd for C₁₇H₁₅Cl₂N₅O₂ 391.0603, found 391.0603.

### Example 16

### N-(2-Amino-2-oxoethyl)-6-(3,4-dichlorobenzyl)pyrazolo[1,5-α]pyrimidine-3-carboxamide

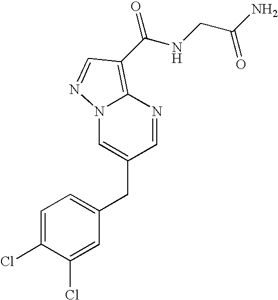

A solution of glycineamide hydrochloride (8 mg, 0.08 mmol) in DMF (0.5 mL) was added to solid 6-(3,4-dichlorobenzyl)pyrazolo[1,5-α]pyrimidine-3-carboxylic acid (INTERMEDIATE 8, 32.2 mg, 0.100 mmol) in a tube and the suspension was stirred at r.t. Triethylamine (21 μL, 0.15 mmol) and a solution of TBTU (48 mg, 0.15 mmol) in DMF (1 mL) were added to the suspension and stirring continued for 30 min at r.t. The reaction mixture was concentrated and purified by preparative HPLC (XTerra C18, 50 mM NH₄HCO₃ pH 10—CH₃CN) to give the title compound (2 mg, 7%) as a white solid. MS (ESI+) calcd for C₁₆H₁₃Cl₂N₅O₂ 377.0446, found 377.0439.

### Intermediate 16

### tert-Butyl[2-({[6-(4-bromobenzyl)pyrazolo[1,5-α]pyrimidin-3-yl]carbonyl}amino)-ethyl]carbamate

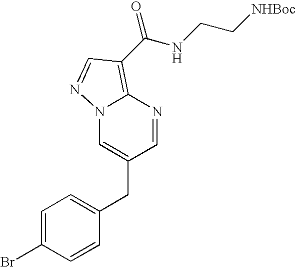

A solution of 6-(4-bromobenzyl)pyrazolo[1,5-α]pyrimidine-3-carboxylic acid (INTERMEDIATE 9, 250 mg, 0.750 mmol) in DMF (15 mL) was treated with tert-butyl (2-aminoethyl)carbamate (140 mg, 0.900 mmol) followed by TBTU (310 mg, 0.0980 mmol) and triethylamine (100 mg, 0.0980 mmol). The mixture was stirred at r.t. for 45 min and then purified by preparative HPLC (XTerra C18, 50 mM NH₄HCO₃ pH 10—CH₃CN) to give the title compound (315 mg, 89%) as a white solid.

### Intermediate 17

### 2-({[6-(4-Bromobenzyl)pyrazolo[1,5-α]pyrimidin-3-yl]carbonyl}amino)ethanaminium trifluoroacetate

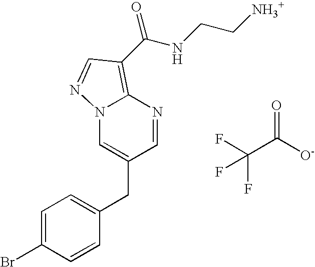

tert-Butyl[2-({[6-(4-bromobenzyl)pyrazolo[1,5-α]pyrimidin-3-yl]carbonyl}amino)ethyl]-carbamate (INTERMEDIATE 16, 315 mg, 0.660 mmol) was dissolved in a mixture of DCM/TFA (5 mL, 50:50) and stirred at r.t. for 30 min. The reaction mixture was concentrated to give the title compound (250 mg, 78%) as a yellow gum. ¹H NMR (400 MHz, CDCl₃) δ ppm 3.37-3.43(m, 2H); 3.80-3.85(m, 2H); 4.05(s, 2H);7.11(d, J=8.53Hz, 2H); 7.50(d, J=8.28Hz, 2H); 8.49-8.54(m, 3H).

### Example 17

### 6-(4-Bromobenzyl)-N-[2-(propionylamino)ethyl]pyrazolo[1,5-α]pyrimidine-3-carboxamide

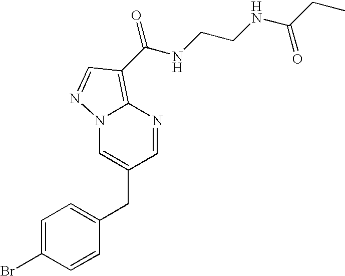

A solution of 2-({[6-(4-bromobenzyl)pyrazolo[1,5-α]pyrimidin-3-yl]carbonyl}amino)-ethanaminium trifluoroacetate (INTERMEDIATE 17, 35 mg 0.094 mmol) in DMF (2 mL) and was treated with propionic acid (8.0 mg, 0.11 mmol) followed by TBTU (39 mg, 0.12 mmol) and triethylamine (12 mg, 0.12 mmol). The mixture was stirred at r.t. overnight and then purified by preparative HPLC (XTerra C18, 50 mM NH₄HCO₃ pH 10—CH₃CN) to give the title compound (9.0 mg, 22%) as a white solid. MS (ESI+) calcd for C₁₉H₂₀BrN₅O₂ 429.0800, found 429.0790.

### Example 18

### 6-(4-Bromobenzyl)-N-{2-[(1H-pyrrol-2-ylcarbonyl)amino]ethyl}pyrazolo[1,5-α]-pyrimidine-3-carboxamide

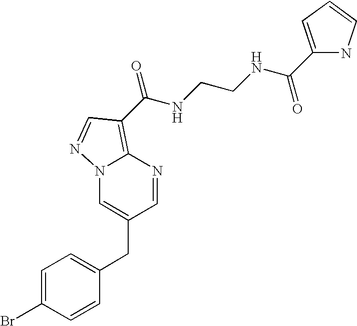

A solution of 2-({[6-(4-bromobenzyl)pyrazolo[1,5-α]pyrimidin-3-yl]carbonyl}amino)-ethanaminium trifluoroacetate (INTERMEDIATE 17, 35 mg 0.094 mmol) in DMF (2 mL) was treated with pyrrole-2-carboxylic acid (12 mg, 0.11 mmol) followed by TBTU (39 mg, 0.12 mmol) and triethylamine (12 mg, 0.12 mmol). The mixture was stirred at r.t. overnight and then purified by preparative HPLC (XTerra C18, 50 mM NH₄HCO₃ pH 10—CH₃CN) to give the title compound (1.6 mg, 4%) as a white solid. MS (ESI+) calcd for C₂₁H₁₉BrN₆O₂ 466.0753, found 466.0753.

### Example 19

### 6-(4-Bromobenzyl)-N-(2-hydroxyethyl)pyrazolo[1,5-α]pyrimidine-3-carboxamide

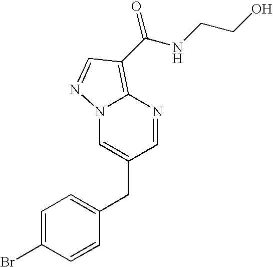

A solution of 6-(4-bromobenzyl)pyrazolo[1,5-α]pyrimidine-3-carboxylic acid (INTERMEDIATE 9, 250 mg, 0.750 mmol) in DMF (15 mL) was treated with 2-aminoethanol (55 mg, 0.90 mmol) followed by TBTU (310 mg, 0.0980 mmol) and triethylamine (100 mg, 0.0980 mmol). The mixture was stirred at r.t. for 45 min and then purified by preparative HPLC (XTerra C18, 50 mM NH4HCO₃ pH 10—CH₃CN) to give the title compound (125 mg, 44%) as a white solid. ¹H NMR (400 MHz, MeOH-d₄) δ ppm 3.59(t, J=10.80 Hz, 2H); 3.74(t, J=11.05 Hz, 2H); 4.13(s, 2H); 7.28(d, J=8.28 Hz, 2H); 7.52(d, J=8.53 Hz, 2H); 8.54(s, 1H); 8.69(s, 1H); 8.92(s, 1H).

### Example 20

### 6-(4-Bromobenzyl)-N-[2-(2,3-dihydroxypropoxy)ethyl]pyrazolo[1,5-α]pyrimidine-3-carboxamide

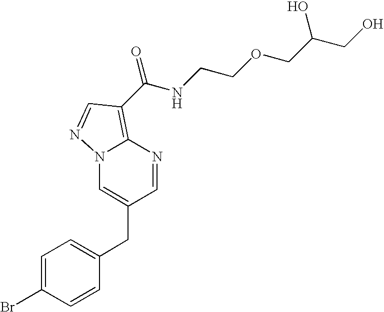

A solution of 6-(4-bromobenzyl)-N-(2-hydroxyethyl)pyrazolo[1,5-α]pyrimidine-3-carboxamide (EXAMPLE 19, 21 mg, 0.057 mmol) in EtOH (2 mL) was treated with KOtBu (12 mg, 0.10 mmol) at r.t. for 15 min upon which oxiran-2-ylmethanol (15 mg, 0.20 mmol) was added. The reaction was heated at 125° C. for 1 h in a microwave reactor and then purified by preparative HPLC (XTerra C18, 50 mM NH₄HCO₃ pH 10—CH₃CN) to give the title compound (4.4 mg, 17%) as a white solid. MS (ESI+) calcd for C₁₉H₂₁BrN₄O₄ 448.0746, found 448.0743.

### Example 21

### 6-(4-Bromobenzyl)-N-(2-methoxyethyl)pyrazolo[1,5-α]pyrimidine-3-carboxamide

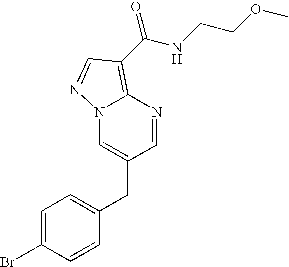

A suspension of 6-(4-bromobenzyl)pyrazolo[1,5-α]pyrimidine-3-carboxylic acid (INTERMEDIATE 9, 30 mg, 0.090 mmol), 2-methoxyethylamine (9.3 μL, 0.11 mmol), N,N-diisopropylethylamine (35 mg, 0.27 mmol) and 1-propanephosphonic acid cyclic anhydride (40 μL, 0.14 mmol) was stirred at 40° C. over night, then at 80° C. for 3 h and purified by preparative HPLC (XTerra C18, 50 mM NH₄HCO₃ pH 10—CH₃CN) to give the title compound as an off-white solid (0.9 mg). MS (ESI+) calcd for C₁₇H₁₇BrN₄O₂ 388.0535, found 388.0532.

### Example 22

### N-[2-(3-Amino-3-oxopropoxy)ethyl]-6-(4-bromobenzyl)pyrazolo[1,5-α]pyrimidine-3-carboxamide

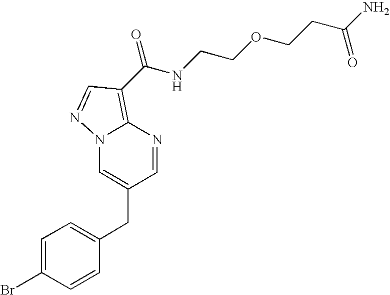

A solution of 6-(4-bromobenzyl)-N-(2-hydroxyethyl)pyrazolo[1,5-α]pyrimidine-3-carboxamide (EXAMPLE 19, 21 mg, 0.057 mmol) in dioxane (2 mL) was cooled on an ice-bath and subsequently treated with 1M KOH (0.12 mL) and acrylamide (7 mg, 0.1 mmol). The mixture was stirred at r.t. overnight and then purified by preparative HPLC (XTerra C18, 50 mM NH4HCO₃ pH 10—CH₃CN) to give the title compound (5 mg, 20%) as a white solid. MS (ESI+) calcd for C₁₉H₂₀BrN₅O₃ 445.0750, found 445.0759.

### Example 23

### 6-(4-Bromobenzyl)-N-[2-(2-cyanoethoxy)ethyl]pyrazolo[1,5-α]pyrimidine-3-carboxamide

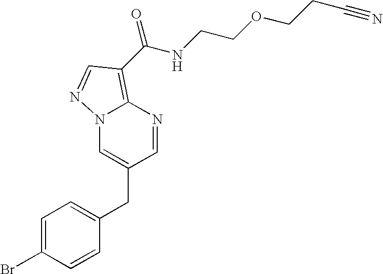

A solution of 6-(4-bromobenzyl)-N-(2-hydroxyethyl)pyrazolo[1,5-α]pyrimidine-3-carboxamide (EXAMPLE 19, 21 mg, 0.057 mmol) in dioxane (2 mL) was cooled on an ice-bath and subsequently treated with 1M KOH (0.06 mL) and acrylnitrile (6 mg, 0.1 mmol). The mixture was stirred at r.t. overnight and then purified by preparative HPLC (XTerra C18, 50 mM NH₄HCO₃ pH 10—CH₃CN) to give the title compound (9.3 mg, 38%) as a beige solid. MS (ESI+) calcd for C₁₉H₁₈BrN₅O₂ 427.0644, found 427.0649.

### Example 24

### 6-(4-Bromobenzyl)-N-[2-(2-hydroxy-2-methylpropoxy)ethyl]pyrazolo[1,5-α]-pyrimidine-3-carboxamide

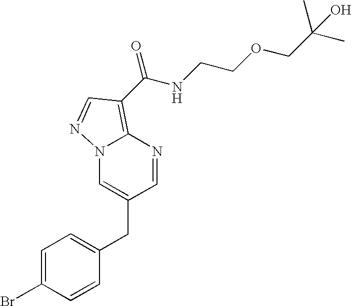

A solution of 6-(4-bromobenzyl)-N-(2-hydroxyethyl)pyrazolo[1,5-α]pyrimidine-3-carboxamide (EXAMPLE 19, 21 mg, 0.057 mmol) in EtOH (2 mL) was treated with KOtBu (12 mg, 0.10 mmol) at r.t. for 15 min upon which 2,2-dimethyloxiran (15 mg, 0.20 mmol) was added. The reaction was heated at 125° C. for 1 h using a microwave reactor and then purified by preparative HPLC (XTerra C18, 50 mM NH₄HCO₃ pH 10—CH₃CN) to give the title compound (4.3 mg, 17%) as a light yellow gum. MS (ESI+) calcd for C₂₀H₂₃BrN₄O₃ 446.0954, found 446.0957.

### Example 25

### 6-(4-Bromobenzyl)-N-[2-(formylamino)ethyl]pyrazolo[1,5-α]pyrimidine-3-carboxamide

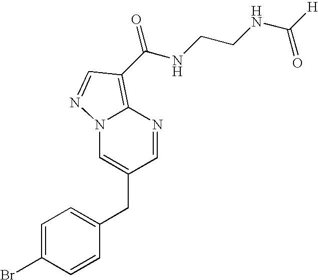

A solution of 2-({[6-(4-bromobenzyl)pyrazolo[1,5-α]pyrimidin-3-yl]carbonyl}amino)-ethanaminium trifluoroacetate (INTERMEDIATE 17, 35 mg 0.094 mmol) in DMF (2 mL) was treated with formic acid (5.0 mg, 0.11 mmol) followed by TBTU (39 mg, 0.12 mmol) and triethylamine (12 mg, 0.12 mmol). The mixture was stirred at r.t. overnight and then purified by preparative HPLC (XTerra C18, 50 mM NH₄HCO₃ pH 10—CH₃CN) to give the title compound (11.4 mg, 30%) as a white solid. Calcd for C₁₇H₁₆BrN₅O₂ 401.0487, found 401.0479.

### EXAMPLE 26

### 6-(4-Bromobenzyl)-N-[2-(glycoloylamino)ethyl]pyrazolo[1,5-α]pyrimidine-3-carboxamide

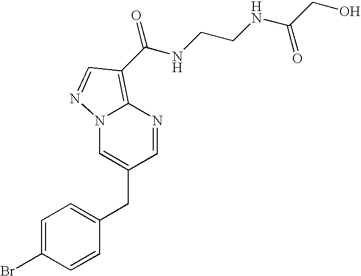

A solution of 2-({[6-(4-bromobenzyl)pyrazolo[1,5-α]pyrimidin-3-yl]carbonyl}amino)-ethanaminium trifluoroacetate (INTERMEDIATE 17, 35 mg 0.094 mmol) in DMF (2 mL) was treated with glycolic acid (8.0 mg, 0.11 mmol) followed by TBTU (39 mg, 0.12 mmol) and triethylamine (12 mg, 0.12 mmol). The mixture was stirred at r.t. overnight and then purified by preparative HPLC (XTerra C18, 50 mM NH₄HCO₃ pH 10—CH₃CN) to give the title compound (18.2 mg, 45%) as a white solid. (ESI+) calcd for C₁₈H₁₈BrN₅O₃ 431.0593, found 431.0592.

### Example 27

### 6-(3-Chloro-4-fluorobenzyl)-N-{[6-(hydroxymethyl)pyridin-2-yl]methyl}pyrazolo[1,5-α]pyrimidine-3-carboxamide

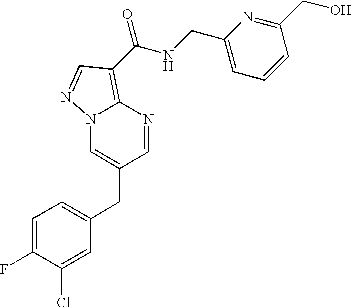

A solution of 6-(3-chloro-4-fluorobenzyl)pyrazolo[1,5-α]pyrimidine-3-carboxylic acid (INTERMEDIATE -10, 25 mg, 0.080 mmol) in DMF (2 mL) was treated with [6-(aminomethyl)pyridin-2-yl]methanol (14 mg, 0.10 mmol) followed by TBTU (34 mg, 0.11 mmol) and triethylamine (11 mg, 0.11 mmol). The reaction was stirred at r.t. overnight and then purified by preparative HPLC (ACE C_{8, 0.1}% TFA—CH₃CN, repurified on XTerra C18, 50 mM NH₄HCO₃ pH 10—CH₃CN) to give the title compound (3.7mg, 11%) as a white solid. MS (ESI+) calcd for C₂₁H₁₇ClFN₅O₂ 425.1055, found 425.1053.

### Example 28

### N-(2-Amino-2-oxoethyl)-6-(3-chloro-4-fluorobenzyl)pyrazolo[1,5-α]pyrimidine-3-carboxamide

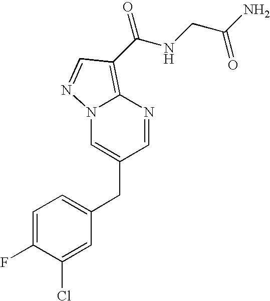

A solution of 6-(3-chloro-4-fluorobenzyl)pyrazolo[1,5-α]pyrimidine-3-carboxylic acid (INTERMEDIATE 10, 31 mg, 0.10 mmol) in DMF (0.5 mL) was treated with TBTU (48 mg, 0.15 mmol) and triethylamine (21 μL, 0.15 mmol) followed by a solution of glycinamide (8.1 mg, 0.070 mmol) in DMF (0.5 mL). The reaction was stirred at r.t. overnight and then purified by preparative HPLC (XTerra C18, 50 mM NH₄HCO₃ pH 10—CH₃CN) to give the title compound (4.5 mg, 12%). MS (ESI+) calcd for C₁₆H₁₃ClFN₅O₂ 361.0742, found 361.0739.

### Example 29

### N-(3-Amino-3-oxopropyl)-6-(3-chloro4-fluorobenzyl)pyrazolo[1,5-α]pyrimidine-3-carboxamide

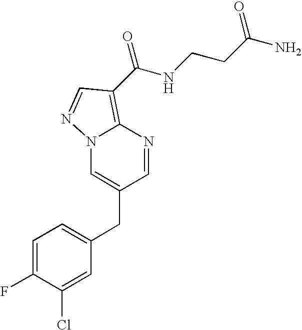

A solution of 6-(3-chloro-4-fluorobenzyl)pyrazolo[1,5-α]pyrimidine-3-carboxylic acid (INTERMEDIATE 10, 31 mg, 0.10 mmol) in DMF (0.5 mL) was treated with TBTU (48 mg, 0.15 mmol) and triethylamine (21 μL, 0.15 mmol) followed by a solution of β-alaninamide (9.7 mg, 0.080 mmol) in DMF (0.5 mL). The reaction was stirred at r.t. overnight and then purified by preparative HPLC (XTerra C18, 50 mM NH₄HCO₃ pH 10—CH₃CN) to give the title compound (3.6 mg, 10%). MS (ESI+) calcd for C₁₇H₁₅ClFN₅O₂ 375.0898, found 375.0891.

### Intermediate 18

### 6-(3-Chloro-4-fluorobenzyl)-N-(2-hydroxyethyl)pyrazolo[1,5-α]pyrimidine-3-carboxamide

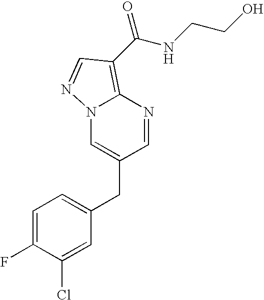

A solution of 6-(3-chloro-4-fluorobenzyl)pyrazolo[1,5-α]pyrimidine-3-carboxylic acid (INTERMEDIATE 10, 1.0 g, 3.3 mmol) in DMF (15 mL) was treated with 2-aminoethanol (242 mg, 4.00 mmol) followed by TBTU (1.3 g, 4.2 mmol) and triethylamine (430 mg, 4.20 mmol). The reaction was stirred at r.t. overnight and then purified by preparative HPLC (ACE C_{8, 0.1}% TFA—CH₃CN) to give the title compound as a light yellow gum (390 mg, 34%).

### Example 30

### 6-(3-Chloro-4-fluorobenzyl)-N-[2-(2-cyanoethoxy)ethyl]pyrazolo 11,5-α]pyrimidine-3-carboxamide

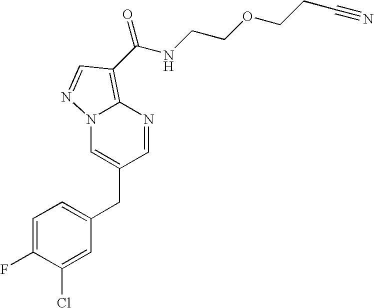

A solution of 6-(3-chloro-4-fluorobenzyl)-N-(2-hydroxyethyl)pyrazolo[1,5-α]pyrimidine-3-carboxamide (INTERMEDIATE 18, 20 mg, 0.057 mmol) in dioxane (2 mL) was cooled on an ice-bath and subsequently treated with 1M KOH (0.06 mL) and acrylnitrile (5 mg, 0.1 mmol). The mixture was stirred at r.t. overnight and then purified by preparative HPLC 1o (ACE C8, 0.1% TFA—CH₃CN, repurified on XTerra C18, 50 mM NH4HCO₃ pH 10—CH₃CN) to give the title compound (0.8 mg, 4%) as an off-white solid. MS (ESI+) calcd for C₁₉H₁₇ClFN₅O₂ 401.1055, found 401.1053.

### Example 31

### 6-[4-Chloro-3-(trifluoromethoxy)benzyl]-N-(2-hydroxyethyl)pyrazolo[1,5-α]-pyrimidine-3-carboxamide

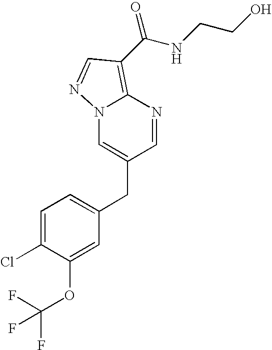

A solution of 6-[4-chloro-3-(trifluoromethoxy)benzyl]pyrazolo[1,5-α]pyrimidine-3-carboxylic acid (INTERMEDIATE 11, 30 mg, 0.072 mmol) in DMF (0.5 mL) was treated with TBTU (39 mg, 0.12 mmol) and N,N-diisopropylethylamine (21 μL, 0.12 mmol) followed by 2-aminoethanol (5.9 mg, 0.096 mmol). The mixture was stirred at r.t. for 3.5 h and purified by preparative HPLC (XTerra C18, 50 mM NH₄HCO₃ pH 10—CH₃CN) to give the title compound (5.6 mg, 17%). MS (ESI+) calcd for C₁₇H₁₄ClF₃N₄O₃ 414.0707, found 414.0707.

### Example 32

### N-(3-Amino-3-oxopropyl)-6-[4-chloro-3-(trifluoromethoxy)benzyl]pyrazolo[1,5-α]-pyrimidine-3-carboxamide

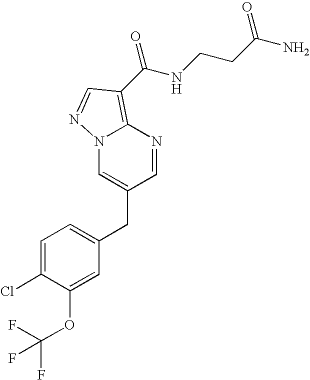

A solution of 6-[4-chloro-3-(trifluoromethoxy)benzyl]pyrazolo[1,5-α]pyrimidine-3-carboxylic acid (INTERMEDIATE 11, 30 mg, 0.072 mmol) in DMF (0.5 mL) was treated with TBTU (39 mg, 0.12 mmol) and N,N-diisopropylethylamine (21 μL, 0.12 mmol) followed by 3-amino-3-oxopropan-1-aminium chloride (12 mg, 0.096 mmol). The mixture was stirred at r.t. for 3.5 h and purified by preparative HPLC (XTerra C18, 50 mM NH₄HCO₃ pH 10—CH₃CN) to give the title compound (5.0 mg, 14%). MS (ESI+) calcd for C₁₈H₁₅ClF₃N₅O₃ 441.0816, found 441.0819.

### Example 33

### N-(2-Amino-2-oxoethyl)-6-[4-chloro-3-(trifluoromethyl)benzyl]pyrazolo[1,5-α]-pyrimidine-3-carboxamide

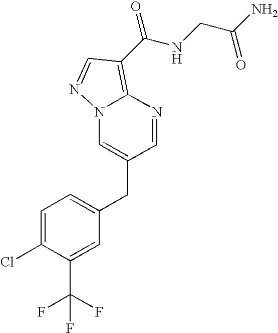

A solution of 6-[4-chloro-3-(trifluoromethyl)benzyl]pyrazolo[1,5-α]pyrimidine-3-carboxylic acid (INTERMEDIATE 12, 28 mg, 0.068 mmol) in DMF (0.5 mL) was treated with TBTU (39 mg, 0.12 mmol) and N,N-diisopropylethylamine (21 μL, 0.12 mmol) followed by glycinamide (11 mg, 0.096 mmol). The mixture was stirred at r.t. for 3.5 h and purified by preparative HPLC (XTerra C18, 50 mM NH₄HCO₃ pH 10—CH₃CN) to give the title compound (3.9 mg, 12%). MS (ESI+) calcd for C₁₇H₁₃ClF₃N₅O₂ 411.0710, found 411.0696.

### Intermediate 19

### 6-[4-Fluoro-3-(trifluoromethyl)benzyl]pyrazolo[1,5-α]pyrimidine-3-carboxylic acid

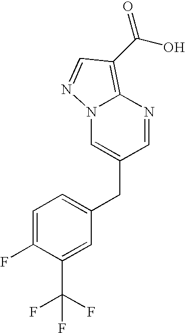

Crude dimethyl[4-fluoro-3-(trifluoromethyl)benzyl]malonate (INTERMEDIATE 6, 0.66 g, 2.1 mmol) was dissolved in dry DCM (7 mL) and the solution cooled to −78° C. Diisobutylaluminium hydride (7 mL, 1M in hexanes) was added to the pre-cooled solution during 2 h. After completed addition ethyl 3-amino-1H-pyrazole-4-carboxylate (0.37 g, 2.4 mmol, 4.8 mL of a 0.50M solution in EtOH) was added dropwise. A gentle stream of N₂ was applied and the reaction mixture was heated at 40° C. to evaporate the solvents. After completed evaporation EtOH (7 mL) and concentrated HCl (0.5 mL) were added and the reaction mixture was heated at 110° C. overnight. After 17 h additional concentrated HCl (440 μL) was added and heating was continued at 110° C. After additional 6 h the reaction was complete and 1M KOH (7 mL) was added and the reaction stirred at 90° C. overnight. More 1M KOH was added after 20 h to adjust the pH to 9 and heating was continued. After 5 h the pH was further adjusted to 14 and the reaction stirred at 90° C. overnight. The reaction mixture was cooled and acidified and the suspension was subjected to centrifugation to isolate the solid product. The solids were washed with toluene and water and centrifuged after each washing. Remaining water was co-evaporated with toluene and the obtained solid dried over night in a vacuum oven to give the title compound (0.83 g, 73% purity, 73%) as an off-white powder.

### Example 34

### N-[2-(Acetylamino)ethyl]-6-[4-fluoro-3-(trifluoromethyl)benzyl]pyrazolo[1,5-α]-pyrimidine-3-carboxamide

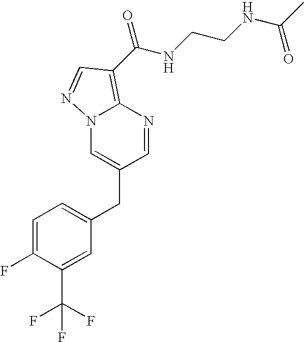

A solution of N-(2-aminoethyl)acetamide (11 mg, 0.11 mmol) in DMF (0.5 mL) was added to solid 6-[4-fluoro-3-(trifluoromethyl)benzyl]pyrazolo[1,5-α]pyrimidine-3-carboxylic acid (INTERMEDIATE 19, 33.9 mg, 0.100 mmol) in a tube and the suspension was stirred at r.t. Triethylamine (21 μL, 0.15 mmol) and a solution of TBTU (48 mg, 0.15 mmol) in DMF (1 mL) were added to the suspension and stirring continued for 30 min at r.t. The reaction mixture was concentrated and purified by preparative HPLC (XTerra C18, 50 mM NH₄HCO₃ pH 10—CH₃CN) to give the title compound (1.1 mg, 3%) as a white solid. MS (ESI+) calcd for C₁₉H₁₇F₄N₅O₂ 423.1318, found 423.1314.

### Example 35

### N-(2-Amino-2-oxoethyl)-6-[4-fluoro-3-(trifluoromethyl)benzyl]pyrazolo[1,5-α]-pyrimidine-3-carboxamide

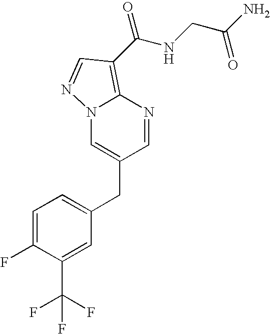

A solution of 6-[4-fluoro-3-(trifluoromethyl)benzyl]pyrazolo[1,5-α]pyrimidine-3-carboxylic acid (INTERMEDIATE 19, 400 mg, 1.18 mmol) and glycinamide hydrochloride (143 mg, 1.30 mmol) in DMF (4 mL) was treated with TBTU (454 mg, 1.42 mmol) and N,N-diisopropylethylamine (315 mg, 425 μL, 2.85 mmol) and the mixture was stirred at r.t. for 30 min. The reaction mixture was then poured on 1M HCl (100 mL) and EtOAc (150 mL), shaked and separated. The organic phase was washed with 1 M HCl (100 mL) and sat. Na₂CO₃ (3×100 mL), dried (Na₂SO₄) and concentrated under reduced pressure. The crude product was purified on silica (10-30% MeOH in EtOAc). Pure fractions were evaporated to give the title compound (404 mg, 87%) as a white solid. MS (ESI+) calcd for C₁₇H₁₃F₄N₅O₂ 395.1005, found 395.1002.

### Example 36

### N-(3-Amino-3-oxopropyl)-6-{1-[3-(trifluoromethyl)phenyl]ethyl}pyrazolo[1,5-α]-pyrimidine-3-carboxamide

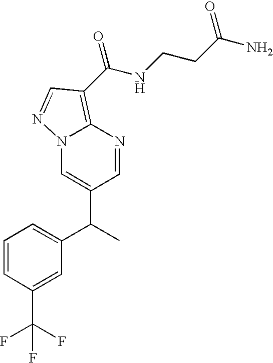

A solution of 6-{1-[3-(trifluoromethyl)phenyl]ethyl}pyrazolo[1,5-α]pyrimidine-3-carboxylic acid (INTERMEDIATE 14, 13.6 mg, 0.0406 mmol) in DMF (0.25 mL) was treated with TBTU (19.5 mg, 0.0607 mmol) and N,N-diisopropylethylamine (10.5 μL, 0.0603 mmol) followed by 3-amino-3-oxopropan-1-aminium chloride (5.4 mg, 0.043 mmol). The mixture was stirred at r.t. overnight and purified by preparative HPLC (XTerra C18, 50 mM NH₄HCO₃ pH 10—CH₃CN) to give the title compound (6.4 mg, 40%). MS (ESI+) calcd for C₁₉H₁₈F₃N₅O₂ 405.1413, found 405.1409.

### Intermediate 20

### Ethyl 6-benzyl-7-hydroxy-5-methylpyrazolo[1,5-α]pyrimidine-3-carboxylate

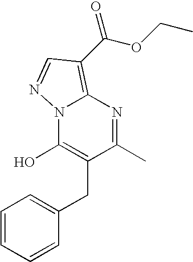

A solution of ethyl 2-benzyl-3-oxobutanoate (220 mg, 1.00 mmol) and ethyl 3-amino-1H-pyrazole-4-carboxylate (155 mg, 1.00 mmol) in HOAc (5 mL) was heated at reflux for 30 min, cooled to r.t. and diluted with water. The formed precipitate was isolated by filtration to give the title compound (165 mg, 53%) as a light yellow solid.

### Intermediate 21

### Ethyl 6-benzyl-7-chloro-5-methylpyrazolo[1,5-α]pyrimidine-3-carboxylate

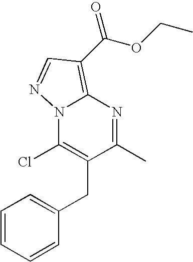

A solution of crude ethyl 6-benzyl-7-hydroxy-5-methylpyrazolo[1,5-α]pyrimidine-3-carboxylate (INTERMEDIATE 20, 150 mg, 0.500 mmol) in phosphoryl chloride (10 mL) was treated with N,N-dimethylaniline (236 mg, 1.90 mmol) and the mixture heated at reflux for 5 h, then cooled and slowly poured on ice-water. The aqueous phase was extracted with CHCl₃, the phases separated and the organic phase concentrated to give the title compound (160 mg, 97%) as a blue-violet gum.

### Intermediate 22

### Ethyl 6-benzyl-5-methylpyrazolo[1,5-α]pyrimidine-3-carboxylate

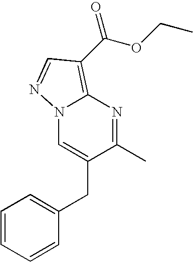

A solution of crude ethyl 6-benzyl-7-chloro-5-methylpyrazolo[1,5-α]pyrimidine-3-carboxylate (INTERMEDIATE 21, 160 mg, 0.480 mmol) in HOAc (10 mL) was treated with NaOAc (205 mg, 2.50 mmol) and Pd/C (5%, 10 mg) and the solution stirred under an H₂ atmosphere at r.t. for 4 h. The crude mixture was filtrated through celite and concentrated, the obtained residue redissolved in EtOH (5 mL) and rearomatized with DDQ (10 mg) during 30 min at r.t. The crude product mixture was purified by preparative HPLC (ACE C_{8, 0.1}% TFA—CH₃CN) to give the title compound (35 mg, 25%) as a blue solid.

### Intermediate 23

### 6-benzyl-5-methylpyrazolo[1,5-α]pyrimidine-3-carboxylic acid

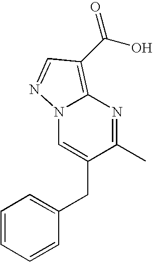

A solution of ethyl 6-benzyl-5-methylpyrazolo[1,5-α]pyrimidine-3-carboxylate (INTERMEDIATE 22, 35 mg, 0.12 mmol) in EtOH (7 mL) was treated with 1M KOH (5 mL) and heated at 70° C. for 15 min. The reaction mixture was concentrated to give the title compound (along with some salts, 30 mg) as a light yellow gum.

### Example 37

### 6-Benzyl-N-[2-(2-hydroxyethoxy)ethyl]-5-methylpyrazolo[1,5-α]pyrimidine-3-carboxamide

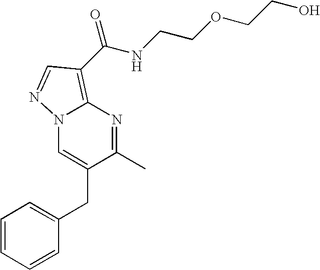

A solution of crude 6-benzyl-5-methylpyrazolo[1,5-α]pyrimidine-3-carboxylic acid (INTERMEDIATE 23, 15 mg, ca. 0.056 mmol) in DMF (2 mL) was treated with 2-(2-aminoethoxy)ethanol (7.0 mg, 0.067 mmol) followed by TBTU (23 mg, 0.073 mmol) and triethylamine (7.4 mg, 0.073 mmol). The mixture was stirred at r.t. overnight and purified by preparative HPLC (XTerra C18, 50 mM NH₄HCO₃ pH 10—CH₃CN) to give the title compound (1.2 mg) as a light yellow gum. MS (ESI+) calcd for C₁₉H₂₂N₄O₃ 354.1692, found 354.1692.

### Intermediate 24

### Ethyl 2-[4-fluoro-3-(trifluoromethoxy)benzyl]-3-oxobutanoate

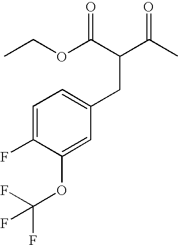

A solution of ethyl 3-oxobutanoate (390 mg, 3.00 mmol) in dry THF (15 mL) was cooled on an ice-bath and carefully treated with NaH (144 mg, 3.60 mmol, 60% in mineral oil). The reaction mixture was warmed to r.t. and stirred for 45 min. 4-(Bromomethyl)-1-fluoro-2-(trifluoromethoxy)benzene (983 mg, 3.60 mmol) was added, it was warmed to 60° C. and stirring continued for 1.5 h. The mixture was cooled to r.t. and quenched with sat. NH₄Cl (100 mL). It was extracted with Et₂O (3×50 mL), the combined org. phases dried (Na₂SO₄) and concentrated to give the title compound (1.16 g, quant.) as a clear oil.

### Intermediate 25

### Ethyl 6-[4-fluoro-3-(trifluoromethoxy)benzyl]-7-hydroxy-5-methylpyrazolo[1,5-α]-pyrimidine-3-carboxylate

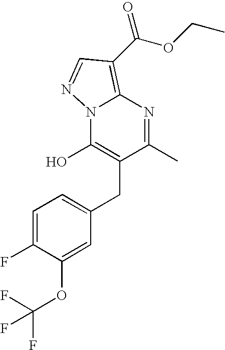

A solution of ethyl 2-[4-fluoro-3-(trifluoromethoxy)benzyl]-3-oxobutanoate (INTERMEDIATE 24, 290 mg, 0.900 mmol) and ethyl 3-amino-1H-pyrazole-4-carboxylate (140 mg, 0.900 mmol) in HOAc (5 mL) was heated at reflux for 1 h, cooled to r.t. and diluted with water. The formed precipitate was isolated by filtration to give the title compound (175 mg, 47%) as a light yellow solid.

### Intermediate 26

### Ethyl 7-chloro-6-[4-fluoro-3-(trifluoromethoxy)benzyl]-5-methylpyrazolo[1,5-α]-pyrimidine-3-carboxylate

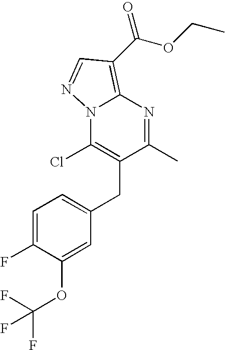

A solution of crude ethyl 6-[4-fluoro-3-(trifluoromethoxy)benzyl]-7-hydroxy-5-methyl-pyrazolo[1,5-α]pyrimidine-3-carboxylate (INTERMEDIATE 25, 175 mg, 0.420 mmol) in phosphoryl chloride (10 mL) was treated with N,N-dimethylaniline (236 mg, 1.90 mmol) and the mixture heated at reflux overnight, then cooled and concentrated under reduced pressure to give the title compound as a blue-violet gum which was directly taken to the next step.

### Intermediate 27

### Ethyl 6-14-fluoro-3-(trifluoromethoxy)benzyl]-5-methylpyrazolo[11,5-α]pyrimidine-3-carboxylate

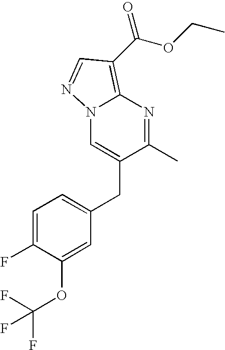

A solution of crude ethyl 7-chloro-6-[4-fluoro-3-(trifluoromethoxy)benzyl]-5-methyl-pyrazolo[1,5-α]pyrimidine-3-carboxylate (INTERMEDIATE 26, ca. 0.420 mmol) in HOAc (15 mL) was treated with NaOAc (75 mg, 0.90 mmol) and Pd/C (5%, 10 mg) and the solution stirred under an H₂ atmosphere at r.t. overnight. The crude mixture was filtrated through celite and concentrated, the obtained residue redissolved in EtOH (5 mL) and rearomatized with DDQ (10 mg) during 30 min at r.t. The crude product mixture was purified by preparative HPLC (ACE C8, 0.1% TFA—CH₃CN) to give the title compound (32 mg, 19% over two steps) as a purple solid.

### Intermediate 28

### 6-[4-Fluoro-3-(trifluoromethoxy)benzyl]-5-methylpyrazolo[1,5-α]pyrimidine-3-carboxylic acid

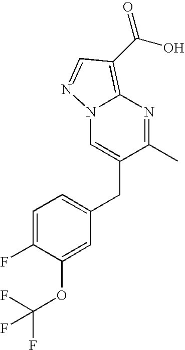

A solution of ethyl 6-[4-fluoro-3-(trifluoromethoxy)benzyl]-5-methylpyrazolo[1,5-α]-pyrimidine-3-carboxylate (INTERMEDIATE 27, 32 mg, 0.080 mmol) in EtOH (7 mL) was treated with 1M KOH (5 mL) and heated at 70° C. for 15 min. The reaction mixture was concentrated to give the title compound (along with some salts, 28 mg) as an orange gum.

### Example 38

### 6-[4-Fluoro-3-(trifluoromethoxy)benzyl]-N-[2-(2-hydroxyethoxy)ethyl]-5-methyl-pyrazolo[1,5-α]pyrimidine-3-carboxamide

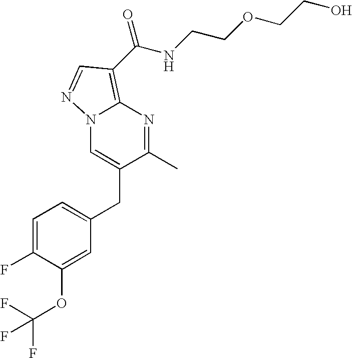

A solution of crude 6-[4-fluoro-3-(trifluoromethoxy)benzyl]-5-methylpyrazolo[1,5-α]-pyrimidine-3-carboxylic acid (INTERMEDIATE 28, 14 mg, ca. 0.037 mmol) in DMF (2 mL) was treated with 2-(2-aminoethoxy)ethanol (3.4 mg, 0.032 mmol) followed by TBTU (15 mg, 0.048 mmol) and triethylamine (4.9 mg, 0.048 mmol). The mixture was stirred at r.t. overnight and purified by preparative HPLC (XTerra C18, 50 mM NH₄HCO₃ pH 10—CH₃CN) to give the title compound (0.7 mg) as a light yellow gum. MS (ESI+) calcd for C₂₀H₂₀F₄N₄O₄ 456.1421, found 456.1420.

### Intermediate 29

### 2-Benzylbutane-1,3-diol

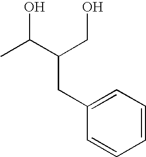

A solution of ethyl 2-benzyl-3-oxobutanoate (220 mg, 1.00 mmol) in EtOH (10 mL) was treated with NaBH₄ (500 mg, 13.2 mmol) and stirred at r.t. overnight. The reaction mixture was poured on sat. NaCl, extracted with EtOAc (3×50 mL), the combined organic phases dried (Na₂SO₄) and concentrated to give the title compound (together with a white solid salt residue, 304 mg) as a clear oil which was directly used in the next step.

### Intermediate 30

### Ethyl 6-benzyl-7-methylpyrazolo[1,5-α]pyrimidine-3-carboxylate

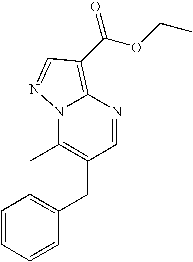

A solution of oxalyl chloride (471 mg, 3.71 mmol) in dry DCM (5 mL) was cooled on an acetone-CO₂₍ₛ₎ bath. DMSO (633 mg, 8.10 mmol) was added over 5 min with evolution of gas. The Swern reagent was allowed to form during 10 min stirring upon which a solution of crude 2-benzylbutane-1,3-diol (INTERMEDIATE 29, ca. 1.0 mmol) in dry DCM/THF (5 mL, 1:1) was added over 5 min and stirring continued for 15 min. Triethylamine (1.71 g, 16.9 mmol) was added over 5 min, the cooling bath removed and water (2 mL) added. After 5 min a solution of ethyl 3-amino-1H-pyrazole-4-carboxylate (410 mg, 2.64 mmol) in EtOH (6 mL) was added and the reaction mixture concentrated. The obtained residue was taken up with EtOH (20 mL) and sat. HCl was added until a pH of 2 was reached (300 μL). The reaction mixture was heated at reflux for 30 min, cooled to r.t. and purified by preparative HPLC (ACE C8, 0.1% TFA—CH₃CN) to give the title compound (140 mg, 47% over two steps) as a slightly brown oil. ¹H NMR (400 MHz, CDCl₃) δ ppm 1.41 (t, J=7.08 Hz, 3 H) 2.80 (s, 3 H) 4.14 (s, 2 H) 4.44 (q, J=7.08 Hz, 2 H) 7.12 (d, J=6.84 Hz, 2 H) 7.23 (t, 1 H) 7.30 (t, J=7.45 Hz, 2 H) 8.57 (s, 1 H) 8.66 (s, 1 H)

### Intermediate 31

### 6-Benzyl-7-methylpyrazolo[1,5-α]pyrimidine-3-carboxylic acid

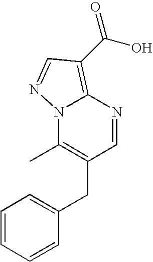

A solution of ethyl 6-benzyl-7-methylpyrazolo[1,5-α]pyrimidine-3-carboxylate (INTERMEDIATE 30, 140 mg, 0.474 mmol) in EtOH (5 mL) was treated with 1M KOH (5 mL) and heated at 75° C. for 30 min. The reaction mixture was poured into 1M HCl (20 mL) and cooled to 15° C. The formed precipitate was isolated by filtration and dried to give the title compound (99 mg, 81%) as a slightly pink solid.

### Example 39

### 6-Benzyl-N-[2-(2-hydroxyethoxy)ethyl]-7-methylpyrazolo[1,5-α]pyrimidine-3-carboxamide

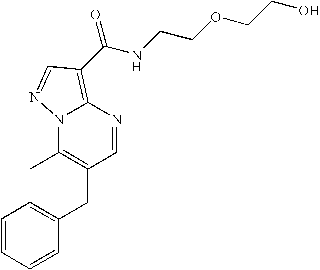

A solution of 6-benzyl-7-methylpyrazolo[1,5-α]pyrimidine-3-carboxylic acid (INTERMEDIATE 31, 10.7 mg, ca. 0.0400 mmol) in DMF (0.3 mL) was treated with 1-propanephosphonic acid cyclic anhydride (48 mg, 0.075 mmol, 50% in EtOAc) and N,N-diisopropylethylamine (19 mg, 0.150 mmol) for a few minutes followed by a solution of 2-(2-aminoethoxy)ethanol (6.3 mg, 0.060 mmol) CH₃CN (0.3 mL). The reaction mixture was stirred at r.t. over the weekend and purified by preparative HPLC (XTerra C18, 50 mM NH₄HCO₃ pH 10—CH₃CN) to give the title compound (1.2 mg, 8% yield). MS (ESI+) calcd for C₁₉H₂₂N₄O₃ 354.1692, found 354.1693.

### Intermediate 32

### Ethyl 3-oxo-2-[3-(trifluoromethyl)benzyl]butanoate

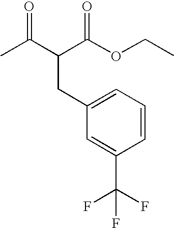

A solution of ethyl 3-oxobutanoate (390 mg, 3.00 mmol) in dry THF (15 mL) was cooled on an ice-bath and carefully treated with NaH (144 mg, 3.60 mmol, 60% in mineral oil). The reaction mixture was warmed to r.t. and stirred for 45 min. 1-(Bromomethyl)-3-(trifluoromethyl)benzene (861 mg, 3.60 mmol) was added, it was warmed to 60° C. and stirring continued for 1.5 h. The mixture was cooled to r.t. and quenched with sat. NH₄Cl (100 mL). It was extracted with Et₂O (3×50 mL), the combined org. phases dried (Na₂SO₄) and concentrated to give the title compound (865 mg, quant.) as a clear oil.

### Intermediate 33

### 2-[3-(Trifluoromethyl)benzyl]butane-1,3-diol

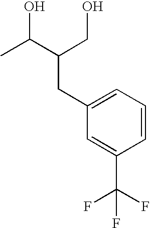

A solution of crude ethyl 3-oxo-2-[3-(trifluoromethyl)benzyl]butanoate (INTERMEDIATE 32, 865 mg, 3.00 mmol) in EtOH (10 mL)was treated with NaBH₄ (300 mg, 7.93 mmol) and stirred at r.t. overnight. The reaction mixture was poured on sat. NaCl, extracted with EtOAc (3×50 mL), the combined organic phases dried (Na₂SO₄) and concentrated to give the title compound (together with a solid salt residue, 388 mg) as a clear oil which was directly used in the next step.

### Intermediate 34

### Ethyl 7-methyl-6-[3-(trifluoromethyl)benzyl]pyrazolo[1,5-α]pyrimidine-3-carboxylate

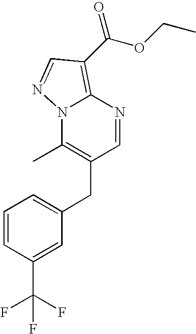

A solution of oxalyl chloride (436 mg, 3.40 mmol) in dry DCM (5 mL) was cooled on an EtOH-CO₂₍ₛ₎ bath. A solution of dry DMSO (586 mg, 7.50 mmol) in DCM (2 mL) was added over 5 min. The Swern reagent was allowed to form during 10 min stirring upon which a solution of crude 2-[3-(trifluoromethyl)benzyl]butane-1,3-diol (INTERMEDIATE 33, 388mg, ca. 1.56mmol) in dry DCM/THF (5mL, 1:1) was added over 5min and stirring continued for 15 min. Triethylamine (1.58 g, 16.0 mmol) was added over 5 min, the cooling bath removed and water (2 mL) added. After 5 min ethyl 3-amino-1H-pyrazole-4-carboxylate (267 mg, 1.72 mmol) was added and the reaction mixture concentrated. The obtained yellow solid was taken up with EtOH (20 mL) and sat. HCl was added in portions of 100 μL until a pH of 2 was reached. The reaction mixture was stirred at r.t. over weekend and purified by preparative HPLC (ACE C8, 0.1% TFA—CH₃CN) to give the title compound (58 mg, 10%) as an off-white solid.

### Intermediate 35

### 7-Methyl-6-[3-(trifluoromethyl)benzyl]pyrazolo[1,5-α]pyrimidine-3-carboxylic acid

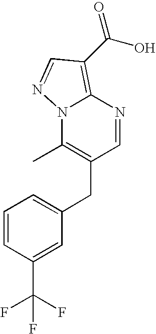

A solution of ethyl 7-methyl-6-[3-(trifluoromethyl)benzyl]pyrazolo[1,5-α pyrimidine-3-carboxylate (INTERMEDIATE 34, 58 mg, 0.16 mmol) in EtOH (3 mL) was treated with 1M KOH (3 mL) and heated at 75° C. for 2 h. The reaction mixture was treated with 1M HCl, the formed precipitate isolated by filtration and dried to give the title compound (45 mg, 84%) as a white solid.

### Example 40

### N-12-(2-Hydroxyethoxy)ethyl]-7-methyl-6-[3-(trifluoromethyl)benzyl]pyrazolo[1,5-α]-pyrimidine-3-carboxamide

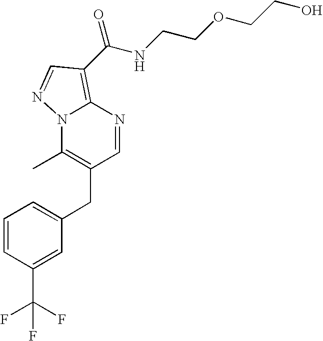

A solution of 7-methyl-6-[3-(trifluoromethyl)benzyl]pyrazolo[1,5-α]pyrimidine-3-carboxylic acid (INTERMEDIATE 35, 13.4 mg, ca. 0.0400 mmol) in DMF (0.3 mL) was treated with 1-propanephosphonic acid cyclic anhydride (48 mg, 0.075 mmol, 50% in EtOAc) and N,N-diisopropylethylamine (19 mg, 0.150 mmol) for a few minutes followed by a solution of 2-(2-aminoethoxy)ethanol (6.3 mg, 0.060 mmol) CH₃CN (0.3 mL). The reaction mixture was stirred at r.t. over weekend and purified by preparative HPLC (XTerra C18, 50 mM NH₄HCO₃ pH 10—CH₃CN) to give the title compound (1.6 mg, 9%) as a white solid. MS (ESI+) calcd for C₂₀H₂₁F₃N₄O₃ 422.1566, found 422.1575.

### Intermediate 36

### Ethyl 3-oxo-2-[3-(trifluoromethoxy)benzyl]butanoate

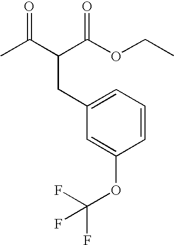

NaH (0.19 g, 4.8 mmol) was weighed into a large Stem-block tube and washed with dry hexane (25 mL). Dry THF (15 mL) was added and the suspension cooled on an ice-bath. Ethyl 3-oxobutanoate (0.52 g, 4.0 mmol) was added slowly under hydrogen evolution and the reaction mixture allowed to stir for a few minutes until a clear solution was obtained. 1-(Bromomethyl)-3-(trifluoromethoxy)benzene (1.02 g, 4.00 mmol) was added and the reaction mixture heated at 65° C. for 1 h. The reaction mixture was poured on sat. NH₄Cl (100 mL) and EtOAc (100 mL), shaked and the phases allowed to separate. The aqueous phase was extracted with EtOAc (2×75 mL), the combined organic phases dried (Na₂SO₄) and concentrated to give the title compound as a clear oil which was directly used in the following step.

### Intermediate 37

### 2-[3-(Trifluoromethoxy)benzyl]butane-1,3-diol

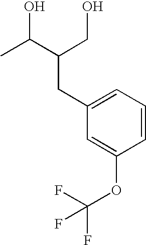

Pellets of LiAlH₄ (0.767 g, 20.2 mmol) were ground into a fine grey suspension by stirring in dry THF (15 mL) for 1 h. The suspension was transferred to a large Stem-block tube. The vessel was cooled on an ice-bath and treated dropwise with a solution of ethyl 3-oxo-2-[3-(trifluoromethoxy)benzyl]butanoate (INTERMEDIATE 36, ca. 4 mmol) in dry THF (5 mL). The reaction mixture was stirred at r.t. overnight and then cooled on an ice-bath. 1M KOH was added dropwise until a white suspension had been obtained. The quenched reaction mixture was diluted with sat. NaCl (100 mL) and extracted with DCM (5×75 mL). The combined organic phases were dried (Na₂SO₄) and concentrated to give the title compound (903 mg, 85% over two steps) as a clear oil.

### Intermediate 38

### Ethyl 7-methyl-6-[3-(trifluoromethoxy)benzyl]pyrazolo[1,5-α]pyrimidine-3-carboxylate

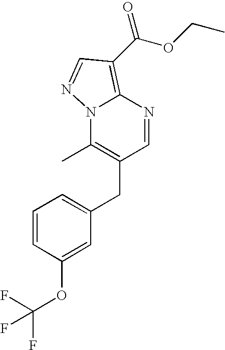

A solution of oxalyl chloride (1.1 g, 8.5 mmol) in dry DCM (15 mL) was cooled on a acetone-CO₂₍ₛ₎ bath. Dry DMSO (1.3 g, 16 mmol) in dry DCM (5 mL) was added over 5 min and left to stir for 10 min. A solution of 2-[3-(trifluoromethoxy)benzyl]butane-1,3-diol (INTERMEDIATE 37, 903 mg, 3.42 mmol) in dry DCM/THF (20 mL, 1:1) was added to the Swern reagent over 5 min and left to stir for 15 min. Dry triethylamine (3.8 g, 38 mmol) was added over 5 min and the reaction mixture removed from the cooling bath. At rt, water (2 ml) was added to quench any remaining Swern reagent, giving a clear two-phasic solution. Ethyl 3-amino-1H-pyrazole-4-carboxylate (0.58 g, 3.7 mmol) was added and the solvents evaporated (not completely, to avoid polymerization of the dicarbonyl compound). The residue was dissolved in EtOH (25 mL) and sat. HCl added until pH 1 was reached (˜0.5 mL). The reaction mixture was stirred at r.t. for 1 h, heated at 75° C. overnight and then diluted with EtOAc (100 mL). The organic phase was washed with 1M HCl (3×100 mL), dried (Na₂SO₄) and concentrated to an orange-brown oil. The crude product was purified on silica (50-70% EtOAc in hexane) to give the title compound as a pale yellow, slowly solidifying oil which was directly used in the next step.

### Intermediate 39

### 7-Methyl-6-[3-(trifluoromethoxy)benzyl]pyrazolo 11,5-α]pyrimidine-3-carboxylic acid

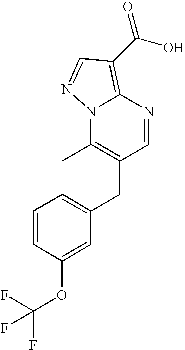

A solution of ethyl 7-methyl-6-[3-(trifluoromethoxy)benzyl]pyrazolo[1,5-α]pyrimidine-3-carboxylate (INTERMEDIATE 38, ca. 3.42 mmol) in EtOH (5 mL) was treated with 1M KOH (5 mL), darkening the reaction mixture. The reaction mixture was heated to reflux for 1 h and then poured on 1M HCl (100 mL) and EtOAc (100 mL). The phases were separated, the organic phase washed with 1M HCl (2×100 mL), dried (Na₂SO₄) and concentrated to give the title compound (160 mg, 13% over 3 steps) as crystallizing needles from yellow oil which were used in following step (EXAMPLE 41) without further purification.

### Example 41

### N-(3-Amino-3-oxopropyl)-7-methyl-6-[3-(trifluoromethoxy)benzyl]pyrazolo[1,5-α]-pyrimidine-3-carboxamide

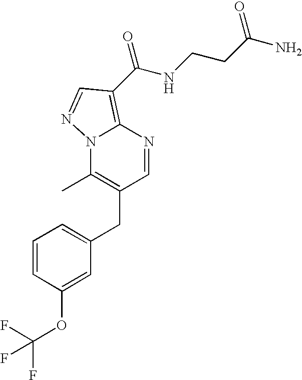

7-Methyl-6-[3-(trifluoromethoxy)benzyl]pyrazolo[1,5-α]pyrimidine-3-carboxylic acid (INTERMEDIATE 39, 14 mg, 0.040 mmol), 3-amino-3-oxopropan-1-aminium chloride (7.5 mg, 0.060 mmol), TBTU (15 mg, 0.048 mmol) and N,N-diisopropylethylamine (16 mg, 0.12 mmol) were dissolved in dry DMF (0.3 mL) and left to stand overnight. The reaction mixture was diluted with MeOH (1.2 mL), filtered and purified by preparative HPLC (Xbridge C18, 50 mM NH₄HCO₃ pH 10—CH₃CN) to give the title compound (6.4 mg, 38%) as a white solid. (ESI+) calcd for C₁₉H₁₈F₃N₅O₃ 421.1362, found 421.1358.

### Intermediate 40

### Ethyl 2-[4-chloro-3-(trifluoromethoxy)benzyl]-3-oxobutanoate

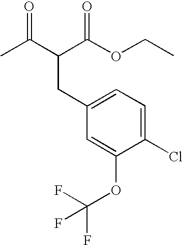

NaH (0.19 g, 4.8 mmol) was weighed into a large Stem-block tube and washed with dry hexane (25 mL). Dry THF (15 mL) was added and the suspension cooled on an ice-bath. Ethyl 3-oxobutanoate (0.52 g, 4.0 mmol) was added slowly under hydrogen evolution and the reaction mixture allowed to stir for a few minutes until a clear solution was obtained. 4-(Bromomethyl)-1-chloro-2-(trifluoromethoxy)benzene (1.16 g, 4.00 mmol) was added and the reaction mixture heated at 65° C. for 1 h. The reaction mixture was poured on sat. NH₄Cl (100 mL) and EtOAc (100 mL), shaked and the phases allowed to separate. The aqueous phase was extracted with EtOAc (2×75 mL), the combined organic phases dried (Na₂SO₄) and concentrated to give the title compound as a clear oil which was directly used in the following step.

### Intermediate 41

### 2-14-Chloro-3-(trifluoromethoxy)benzyl]butane-1,3-diol

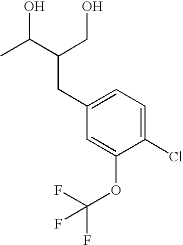

Pellets of LiAlH₄ (0.767 g, 20.2 mmol) were ground into a fine grey suspension by stirring in dry THF (15 mL) for 1 h. The suspension was transferred to a large Stem-block tube. The vessel was cooled on an ice-bath and treated dropwise with a solution of ethyl 2-[4-chloro-3-(trifluoromethoxy)benzyl]-3-oxobutanoate (INTERMEDIATE 40, ca. 4 mmol) in dry THF (5 mL). The reaction mixture was stirred at r.t. overnight and then cooled on an ice-bath. 1M KOH was added dropwise until a white suspension had been obtained. The quenched reaction mixture was diluted with sat. NaCl (100 mL) and extracted with DCM (5×75 mL). The combined organic phases were dried (Na₂SO₄) and concentrated to give the title compound (1.10 g, 92% over two steps) as a clear oil.

### Intermediate 42

### Ethyl 6-[4-chloro-3-(trifluoromethoxy)benzyl]-7-methylpyrazolo[1,5-α]pyrimidine-3-carboxylate

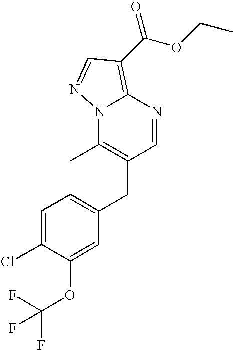

A solution of oxalyl chloride (1.1 g, 8.5 mmol) in dry DCM (15 mL) was cooled on a acetone-CO₂₍ₛ₎ bath. Dry DMSO (1.3 g, 16 mmol) in dry DCM (5 mL) was added over 5 min and left to stir for 10 min. A solution of 2-[4-chloro-3-(trifluoromethoxy)benzyl]butane-1,3-diol (INTERMEDIATE 41, 1.10 g, 3.68 mmol) in dry DCM/THF (20 mL, 1:1) was added to the Swern reagent over 5 min and left to stir for 15 min. Dry triethylamine (3.8 g, 38 mmol) was added over 5 min and the reaction mixture removed from the cooling bath. At rt, water (2 ml) was added to quench any remaining Swern reagent, giving a clear two-phasic solution. Ethyl 3-amino-1H-pyrazole4-carboxylate (0.58 g, 3.7 mmol) was added and the solvents evaporated (not completely, to avoid polymerization of the dicarbonyl compound). The residue was dissolved in EtOH (25 mL) and sat. HCl added until pH 1 was reached (˜0.5 mL). The reaction mixture was stirred at r.t. for 1 h, heated at 75° C. overnight and then diluted with EtOAc (100 mL). The organic phase was washed with 1M HCl (3×100 mL), dried (Na₂SO₄) and concentrated to an orange-brown oil. The crude product was purified on silica (50-70% EtOAc in hexane) to give the title compound as a pale yellow, slowly solidifying oil which was directly used in the next step.

### Intermediate 43

### 6-[4-Chloro-3-(trifluoromethoxy)benzyl]-7-methylpyrazolo[1,5-α]pyrimidine-3-carboxylic acid

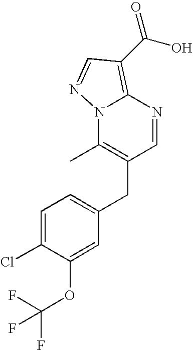

A solution of ethyl 6-[4-chloro-3-(trifluoromethoxy)benzyl]-7-methylpyrazolo[1,5-α]-pyrimidine-3-carboxylate (INTERMEDIATE 42, ca. 3.68 mmol) in EtOH (5 mL) was treated with 1M KOH (5 mL), darkening the reaction mixture. The reaction mixture was heated to reflux for 1 h and then poured on 1M HCl (100 mL) and EtOAc (100 mL). The phases were separated, the organic phase washed with 1M HCl (2×100 mL), dried (Na₂SO₄) and concentrated to give the title compound (155 mg, 12% over 3 steps) as a crystallizing yellow oil which was used in following steps without further purification.

### Example 42

### N-[2-(Acetylamino)ethyl]-6-[4-chloro-3-(trifluoromethoxy)benzyl]-7-methylpyrazolo-[1,5-α]pyrimidine-3-carboxamide

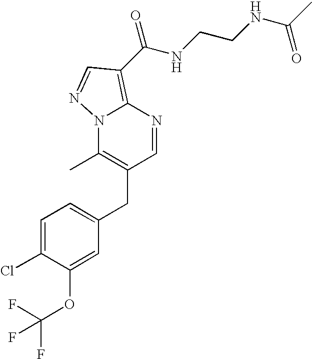

6-[4-Chloro-3-(trifluoromethoxy)benzyl]-7-methylpyrazolo[1,5-α]pyrimidine-3-carboxylic acid (INTERMEDIATE 43, 15 mg, 0.040 mmol), N-(2-aminoethyl)acetamide (6.1 mg, 0.060 mmol), TBTU (15 mg, 0.048 mmol) and N,N-diisopropylethylamine (16 mg, 0.12 mmol) were dissolved in dry DMF (0.3 mL) and left to stand overnight. The reaction mixture was diluted with MeOH (1.2 mL), filtered and purified by preparative HPLC (Xbridge C18, 50 mM NH₄HCO₃ pH 10—CH₃CN) to give the title compound (4.6 mg, 24%) as a white solid. (ESI+) calcd for C₂₀H₁₉ClF₃N₅O₃ 469.1129, found 469.112-4.

### Example 43

### 6-[4-Chloro-3-(trifluoromethoxy)benzyl]-N-12-(2-hydroxyethoxy)ethyl]-7-methyl-pyrazolo[1,5-α]pyrimidine-3-carboxamide

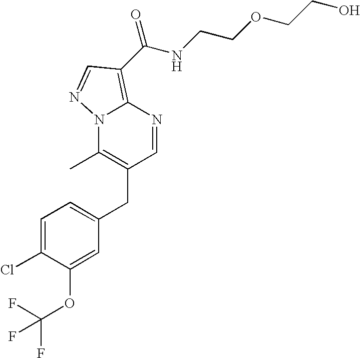

6-[4-Chloro-3-(trifluoromethoxy)benzyl]-7-methylpyrazolo[1,5-α]pyrimidine-3-carboxylic acid (INTERMEDIATE 43, 15 mg, 0.040 mmol), 2-(2-aminoethoxy)ethanol (6.3 mg, 0.060 mmol), TBTU (15 mg, 0.048 mmol) and N,N-diisopropylethylamine (16 mg, 0.12 mmol) were dissolved in dry DMF (0.3 mL) and left to stand overnight. The reaction mixture was diluted with MeOH (1.2 mL), filtered and purified by preparative HPLC (Xbridge C18, 50 mM NH₄HCO₃ pH 10—CH₃CN) to give the title compound (2.0 mg, 11%) as a white solid. (ESI+) calcd for C₂₀H₂₀ClF₃N₄O₄ 472.1125, found 472.1123.

### Intermediate 44

### Ethyl 2-[4-fluoro-3-(trifluoromethoxy)benzyl]-3-oxobutanoate

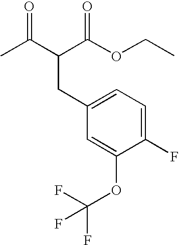

A solution of ethyl 3-oxobutanoate (390 mg, 3.00 mmol) in dry THF (15 mL) was cooled on an ice-bath and carefully treated with NaH (144 mg, 3.60 mmol, 60% in mineral oil). The reaction mixture was stirred for 45 min upon which 4-(bromomethyl)-1-fluoro-2-(trifluoromethyl)benzene (983 mg, 3.60 mmol) was added. It was warmed to 60° C. and stirring continued for 1.5 h. The mixture was cooled to r.t. and quenched with sat. NH₄Cl (100 mL). It was extracted with Et₂O (3×50 mL), the combined org. phases dried (Na₂SO₄) and concentrated to give the title compound (1.16 g, quant.) as a clear oil which was directly used in the next step.

### Intermediate 45

### 2-14-Fluoro-3-(trifluoromethoxy)benzyl]butane-1,3-diol

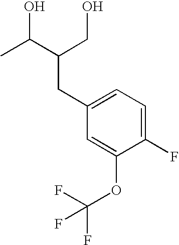

A solution of crude ethyl 2-[4-fluoro-3-(trifluoromethoxy)benzyl]-3-oxobutanoate (INTERMEDIATE 44, ca. 3.00 mmol) in EtOH (10 mL) was treated with NaBH₄ (300 mg, 7.93 mmol) and stirred at r.t. over weekend. The reaction mixture was poured on sat. NaCl, extracted with EtOAc (3×50 mL), the combined organic phases dried (Na₂SO₄) and concentrated to give the title compound (together with a solid residue, 267 mg) as a clear oil which was directly used in the next step.

### Intermediate 46

### Ethyl 6-14-fluoro-3-(trifluoromethoxy)benzyl]-7-methylpyrazolo[1,5-α]pyrimidine-3-carboxylate

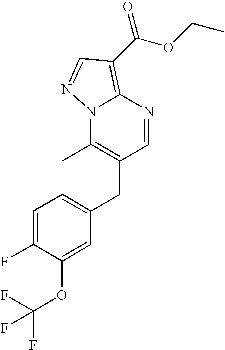

A solution of oxalyl chloride (264 mg, 2.08 mmol) in dry DCM (5 mL) was cooled on an EtOH-CO₂₍ₛ₎ bath. A solution of dry DMSO (355 mg, 4.54 mmol) in DCM (2 mL) was added over 5 min. The Swern reagent was allowed to form during 10 min stirring upon which a solution of crude 2-[4-fluoro-3-(trifluoromethoxy)benzyl]butane-1,3-diol (INTERMEDIATE 45, 267 mg, ca. 0.946 mmol) in dry DCM/THF (5 mL, 1:1) was added over 5 min and stirring continued for 15 min. Triethylamine (0.96 g, 9.6 mmol) was added over 5 min and the cooling bath removed. At rt, water (2 mL) was added to quench any remaining Swern reagent, giving a clear two-phasic solution. Ethyl 3-amino-1H-pyrazole-4-carboxylate (161 mg, 1.04 mmol) was added and the reaction mixture concentrated. The obtained yellow solid was taken up with EtOH (20 mL) and sat. HCl was added in portions of 100 μL until a pH of 2 was reached. The reaction mixture was stirred at r.t. over weekend and purified by preparative HPLC (ACE C8, 0.1% TFA—CH₃CN) to give the title compound (21 mg, 1.8% over 4 steps) as an off-white solid.

### Intermediate 47

### 6-[4-Fluoro-3-(trifluoromethoxy)benzyl]-7-methylpyrazolo[1,5-α]pyrimidine-3-carboxylic acid

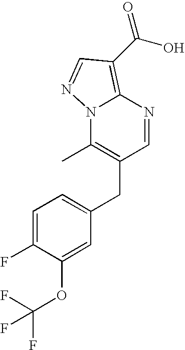

A solution of ethyl 6-[4-fluoro-3-(trifluoromethoxy)benzyl]-7-methylpyrazolo[1,5-α]pyrimidine-3-carboxylate (INTERMEDIATE 46, 21 mg, 0.053 mmol) in EtOH (3 mL) was treated with 1M KOH (3 mL) and heated at 75° C. for 2 h. The reaction mixture was treated with 1M HCl, the formed precipitate isolated by filtration and dried to give the title compound (15 mg, 81%) as a white solid.

### Example 44

### N-[2-(Acetylamino)ethyl]-6-[4-fluoro-3-(trifluoromethoxy)benzyl]-7-methylpyrazolo-[1,5-α]pyrimidine-3-carboxamide

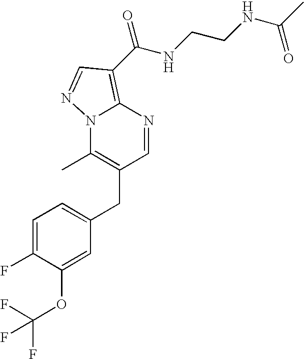

A solution of 6-[4-fluoro-3-(trifluoromethoxy)benzyl]-7-methylpyrazolo[1,5-α]pyrimidine-3-carboxylic acid (INTERMEDIATE 47, 15 mg, ca. 0.040 mmol) in DMF (0.3 mL) was treated with 1-propanephosphonic acid cyclic anhydride (48 mg, 0.075 mmol, 50% in EtOAc) and N,N-diisopropylethylamine (19 mg, 0.15 mmol) for a few minutes followed by a solution of N-(2-aminoethyl)acetamide (6.1 mg, 0.060 mmol) CH₃CN (0.3 mL). The reaction mixture was stirred at r.t. over the weekend and purified by preparative HPLC (XTerra C18, 50 mM NH₄HCO₃ pH 10—CH₃CN) to give the title compound (2.2 mg, 12%) as a white solid. MS (ESI+) calcd for C₂₀H₁₉F₄N₅O₃ 453.142-4, found 453.1427.

### Example 45

### 6-[4-Fluoro-3-(trifluoromethyl)benzyl]-N-(6-methoxypyridin-3-yl)pyrazolo[1,5-α]-pyrimidine-3-carboxamide

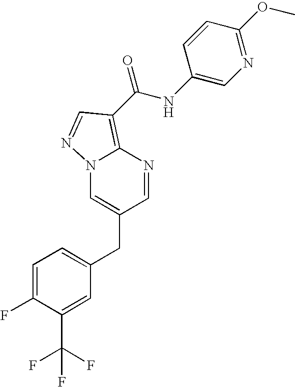

A mixture of 6-[4-fluoro-3-(trifluoromethyl)benzyl]pyrazolo[1,5-α]pyrimidine-3-carboxylic acid (INTERMEDIATE 19, 25.3 mg, 0.075 mmol), 5-amino-2-methoxypyridine (18.5 mg, 0.149 mmol), TBTU (28.7 mg, 0.090 mmol) and N,N-diisopropylethylamine (0.019 ml, 0.112 mmol) in DMF (1 ml) was stirred at r.t. overnight. The crude product was purified by preparative HPLC (XTerra C18, 50 mM NH₄HCO₃ pH 10—CH₃CN) to give the title compound (7.9 mg, 24%) as a white solid. MS (ESI+) calcd for C₂₁H₁₅F₄N₅O₂ 445.1161, found 445.1173.

### Example 46

### 6-[4-Fluoro-3-(trifluoromethyl)benzyl]-N-pyridin-3-ylpyrazolo[1,5-α]pyrimidine-3-carboxamide

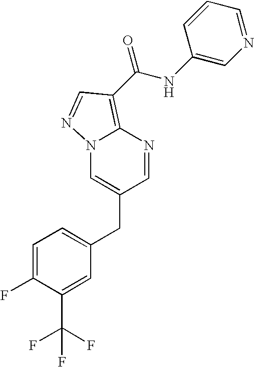

A solution of 6-[4-fluoro-3-(trifluoromethyl)benzyl]pyrazolo[1,5-α]pyrimidine-3-carboxylic acid (INTERMEDIATE 19, 20.0 mg, 0.059 mmol), 3-aminopyridine (22 mg, 0.24 mmol) and triethylamine (32 μL, 0.24 mmol) in DMF (2 ml) was treated with TBTU (76 mg, 0.24 mmol). The reaction mixture was heated at 50° C. overnight and the crude product purified by preparative HPLC (ACE C8, 0.1% TFA—CH₃CN) to give the title compound as a white solid with 70% purity. The white solid was dissolved in DCM and washed with 1M KOH (1×), the organic phase was dried (MgSO₄) and evaporated to give the title compound (1.9 mg, 7.8%) as a white solid. MS (ESI+) calcd for C₂₀H₁₃F₄N₅O 415.1056, found 415.1074.

### Intermediate 48

### 6-[4-Fluoro-3-(trifluoromethyl)benzyl]pyrazolo[1,5-α]pyrimidine-3-carbonyl chloride

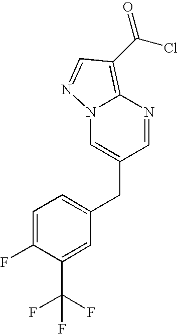

A solution of 6-[4-fluoro-3-(trifluoromethyl)benzyl]pyrazolo[1,5-α]pyrimidine-3-carboxylic acid (INTERMEDIATE 19, 214 mg, 0.63 mmol) in DCM was treated with a solution of oxalyl chloride (160 mg, 1.26 mmol) in DCM and stirred at r.t. for 30 min. The reaction mixture was concentrated to give the title compound (226 mg, quant.) as a yellow solid, which was directly used in the next steps. ¹H NMR (400 MHz, CDCl₃) δ ppm 4.18 (s, 2 H) 7.21-7.28 (m, 1 H) 7.42 (td, J=5.37, 2.20 Hz, 1 H) 7.49 (dd, J=6.35,1.95 Hz, 1 H) 8.53 (s, 1 H) 8.66 (s, 1 H) 8.78 (d, J=2.20 Hz, 1 H)

### Example 47

### 2-(Acetylamino)ethyl 6-[4-fluoro-3-(trifluoromethyl)benzyl]pyrazolo[1,5-α]-pyrimidine-3-carboxylate

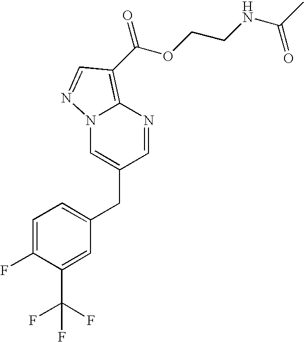

A mixture of 6-[4-fluoro-3-(trifluoromethyl)benzyl]pyrazolo[1,5-α]pyrimidine-3-carbonyl chloride (INTERMEDIATE 48, 20 mg, 0.056 mmol), N-(2-hydroxyethyl)acetamide (11.8 mg, 0.119 mmol) and DMAP (8.2 mg, 0.067 mmol) in DCM was stirred at r.t. overnight. The crude product was purified by preparative HPLC (XTerra C18, 50 mM NH₄HCO₃ pH 10—CH₃CN) to give the title compound (2.7 mg, 11%) as a solid. MS (ESI+) calcd for C₁₉H₁₆F₄N₄O₃ 424.1158, found 424.1161.

### Example 48

### 2-Amino-2-oxoethyl 6-[4-fluoro-3-(trifluoromethyl)benzyl]pyrazolo[1,5-α]pyrimidine-3-carboxylate

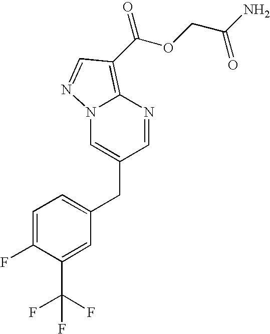

A mixture of 6-[4-fluoro-3-(trifluoromethyl)benzyl]pyrazolo[1,5-α]pyrimidine-3-carbonyl chloride (INTERMEDIATE 48, 20 mg, 0.056 mmol), 2-hydroxyacetamide (8.39 mg, 0.119 mmol) and DMAP (8.2 mg, 0.067 mmol) in DCM was stirred at r.t. overnight. The crude product was purified by preparative HPLC (XTerra C18, 50 mM NH₄HCO₃ pH 10—CH₃CN) to give the title compound (12.4 mg, 55%) as a solid. MS (ESI+) calcd for C₁₇H₁₂F₄N₄O₃ 396.0845, found 396.0845.

### Example 49

### 2-(2-Hydroxyethoxy)ethyl 6-14-fluoro-3-(trifluoromethyl)benzyl]pyrazolo[1,5-α]-pyrimidine-3-carboxylate

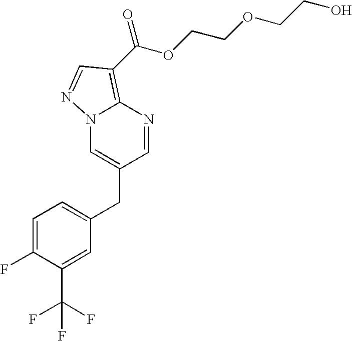

A mixture of 6-[4-fluoro-3-(trifluoromethyl)benzyl]pyrazolo[1,5-α]pyrimidine-3-carbonyl chloride (INTERMEDIATE 48, 30 mg, 0.084 mmol), 2,2′-oxydiethanol (11.9 mg, 0.119 mmol) and DMAP (8.2 mg, 0.067 mmol) in DCM was stirred at r.t. overnight. The crude product was purified by preparative HPLC (XTerra C18, 50 mM NH₄HCO₃ pH 10—CH₃CN) to give the title compound (30.2 mg, 84%) as a solid. MS (ESI+) calcd for Cl₉H₁₇F₄N₃O₄ 427.1155, found 427.1155.

### Intermediate 49

### 6-Bromopyrazolo[1,5-α]pyrimidine-3-carboxylic acid

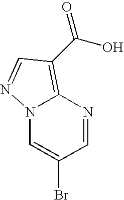

3-Amino-1H-pyrazole-4-carboxylic acid (5.00 g, 39.3 mmol) was mixed with HOAc (30 mL) and bromomalonaldehyde (5.94 g, 39.3 mmol) in ethanol (10 mL). The mixture was heated at 70° C. for 80 min. The reaction mixture was cooled to rt, the formed precipitate filtered off, washed with ethanol and dried to give the title compound (5.89 g, 62%). MS (ESI+) 242, 244 (M+H)⁺.

### Intermediate 50

### N-(2-Amino-2-oxoethyl)-6-bromopyrazolo[1,5-α]pyrimidine-3-carboxamide

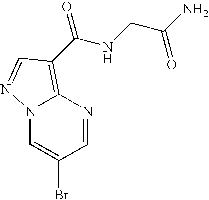

A solution of 6-bromopyrazolo[1,5-α]pyrimidine-3-carboxylic acid (INTERMEDIATE 49, 1.00 g, 4.13 mmol) in DMF (10 ml) was treated with triethylamine (1.9 ml, 14 mmol), TBTU (1.59 g, 4.96 mmol) and glycinamide hydrochloride (0.550 g, 4.96 mmol) and stirred for 2 h at r.t. The formed precipitate was filtered off and washed with acetonitrile to give the title compound (1.31 g, quant.). MS (ESI+) 298, 300 (M+H)⁺.

### Example 50

### N-(2-Amino-2-oxoethyl)-6-{[3-(trifluoromethyl)phenyl]thio}pyrazolo[1,5-α]-pyrimidine-3-carboxamide

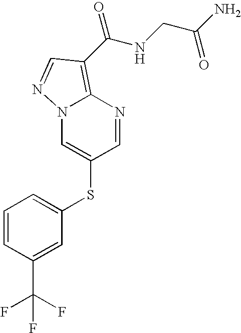

A solution of CuI (0.08 mg) and benzotriazole (0.1 mg) in DMSO (1 mL) was treated with N-(2-amino-2-oxoethyl)-6-bromopyrazolo[1,5-α]pyrimidine-3-carboxamide (INTERMEDIATE 50, 25 mg, 0.084 mmol) and stirred at r.t. for 10 min. 3-(Trifluoromethyl)benzenethiol (15 mg, 0.084 mmol) and KOtBu (13 mg, 0.12 mmol) were added, the reaction mixture warmed to 40° C. and stirred overnight. The crude product was purified by preparative HPLC (ACE C8, 0.1% TFA—CH₃CN) to give the title compound (5 mg, 15%). MS (ESI+) calcd for C₁₆H₁₂F₃N₅O₂S 395.0663, found 395.0668.

### Intermediate 51

### 6-(3,4-Dichlorobenzyl)pyrazolo[1,5-α]pyrimidine

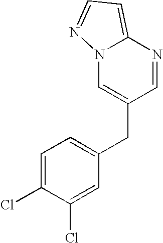

Dimethyl 2-(3,4-dichlorobenzyl)malonate (INTERMEDIATE 1, 3.5 g, 12.0 mmol) was dissolved in dry DCM (70 mL) and cooled to −78° C. Diisobutylaluminium hydride (30 mL, 1M in hexanes) was added dropwise over 2 h. After completed addition the reaction was quenched by dropwise addition (over a period of 20 min) of a solution of 3-aminopyrazole (1.0 g, 12.0 mmol) in MeOH (10 mL) upon which conc. HCl (2 mL) was added. The mixture was allowed to warm to r.t. and concentrated to give a solid. The solid was redissolved in EtOH (100 mL), treated with additional conc. HCl (2 mL) and the mixture stirred at 75° C. for 1 h. The reaction mixture was concentrated and the residue taken up with EtOAc. It was washed with brine, dried (Na₂SO₄) and concentrated to give the crude product as an oil. This material was purified by column chromatography (SiO₂, Hexanes/EtOAc 2:1) to give the title compound (1.5 g, 45%) as a yellow solid. ¹H NMR (400 MHz, CDCl₃) δ ppm 3.99 (s, 2 H) 6.70 (dd, J=2.44, 0.98 Hz, 1 H) 7.07 (dd, J=8.18, 2.08 Hz, 1 H) 7.33 (d, J=2.20 Hz, 1 H) 7.43 (d, J=8.30 Hz, 1 H) 8.10 (d, J=2.44 Hz, 1 H) 8.35 (d, J=2.20 Hz, 1 H) 8.43 (dd, J=2.20, 0.98 Hz, 1 H).

### Intermediate 52

### 6-(3,4-Dichlorobenzyl)-3-nitropyrazolo[1,5-α]pyrimidine

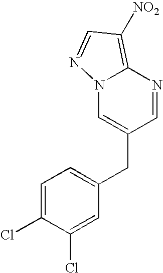

TFFA (0.153 ml, 1.10 mmol) was added dropwise to a solution of 6-(3,4-dichlorobenzyl)pyrazolo[1,5-α]pyrimidine (INTERMEDIATE 51, 153 mg, 0.550 mmol) and tetrabutylammonium nitrate (184 mg, 0.605 mmol) in DCM (5 mL) at 0° C. The mixture was stirred at 0° C. for 30 min and subsequently concentrated to ca 1 mL. This residue was subjected to flash column chromatography (SiO₂, 0-1% MeOH in DCM) to give the title compound (46.1 mg, 26%) as a yellow oil. ¹H NMR (400 MHz, CDCl₃) δ ppm 4.12 (s, 2 H) 7.05 (dd, J=8.18, 2.08 Hz, 1 H) 7.30 (d, J=2.20 Hz, 1 H) 7.43 (d, J=8.30 Hz, 1 H) 8.45-8.54 (m, 1 H) 8.72 (s, 1 H) 8.79 (d, J=2.20 Hz, 1 H).

### Intermediate 53

### 6-(3,4-Dichlorobenzyl)pyrazolo[1,5-α]pyrimidin-3-amine

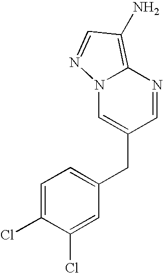

A suspension of 6-(3,4-dichlorobenzyl)-3-nitropyrazolo[1,5-α]pyrimidine (INTERMEDIATE 52, 24 mg, 0.059 mmol) in EtOH (2 mL) and water (0.75 mL) was treated with Fe powder (60 mg) and conc. HCl (20 μL) and heated at 60° C. for 30 min. 2M NaOH (0.105 mL) was added and the mixture filtered through a pad of Celite. The solids were washed several times with THF. The filtrate was concentrated and the crude product purified by preparative HPLC (XTerra C18, 50 mM NH₄HCO₃ pH 10—CH₃CN) to give the title compound (9.7 mg, 56%) as a yellow solid. ¹H NMR (400 MHz, CDCl₃) δ ppm 3.36 (br. s, 2 H) 3.92 (s, 2 H) 7.05 (dd, 1 H) 7.32 (d, J=2.20 Hz, 1 H) 7.42 (d, J=8.30 Hz, 1 H) 7.78 (s, 1 H) 8.10 (d, J=2.20 Hz, 1 H) 8.21 (d, J=2.20 Hz, 1 H).

### Example 51

### 2-cyano-N-[6-(3,4-dichlorobenzyl)pyrazolo[1,5-α]pyrimidin-3-yl]acetamide

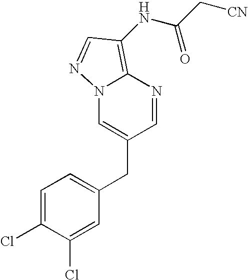

A solution of crude 6-(3,4-dichlorobenzyl)pyrazolo[1,5-α]pyrimidin-3-amine (ca 60% pure; INTERMEDIATE 53, 57.5 mg, 0.196 mmol), cyanoacetic acid (20.0 mg, 0.235 mmol) and 1,3-diisopropylcarbodiimid (29.7 mg, 0.235 mmol) in THF (2 mL) was heated at reflux for 1 h. The crude product was purified by preparative HPLC (XTerra C18, 50 mM NH₄HCO₃ pH 10—CH₃CN) to give the title compound (7.4 mg) as a yellow solid. MS (ESI+) calcd for C₁₆H₁₁Cl₂N₅O 359.0340, found 359.0337.

### Example 52

### N-[6-(3,4-Dichlorobenzyl)pyrazolo[1,5-α]pyrimidin-3-yl]-N′-(2-furylmethyl)urea

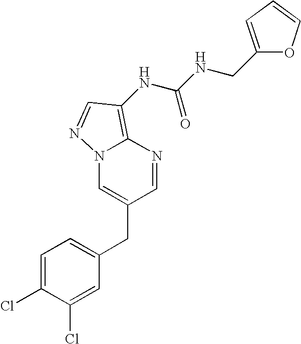

A solution of 6-(3,4-dichlorobenzyl)pyrazolo[1,5-α]pyrimidin-3-amine (INTERMEDIATE 53, 9.9 mg, 0.034 mmol) in DCM (1 mL) was treated with furfuryl isocyanate (4.16 mg, 0.034 mmol) and stirred at r.t. overnight. The crude product was purified by preparative HPLC (XTerra C18, 50 mM NH₄HCO₃ pH 10—CH₃CN) to give the title compound (4.4 mg, 31%) as a yellow solid. MS (ESI+) calcd for C₁₉H₁₅Cl₂N₅O₂ 415.0602, found 415.0604.

### Intermediate 54

### Ethyl 6-bromopyrazolo[1,5-α]pyrimidine-3-carboxylate

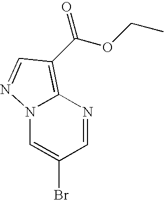

A solution of bromomalonaldehyde (1.00 g, 6.66 mmol) in EtOH (15 mL) at 70° C. was treated with ethyl 3-amino-1H-pyrazole-4-carboxylate (1.04 g, 6.66 mmol) and HOAc (5 mL) and the mixture was stirred at 70° C. for 30 min. DCM (150 mL) and 1M NaOH (30 mL) were added and the phases separated. The aqueous layer was extracted with DCM, the combined organic phases dried and concentrated to give the title compound (1.66 g, 92%) as an off-white solid. ¹H NMR (400 MHz, CDCl₃) δ ppm 1.42 (t, J=7.08 Hz, 3 H) 4.45 (q, J=7.08 Hz, 2 H) 8.55 (s, 1 H) 8.76 (d, 1 H) 8.91 (d, J=2.20 Hz, 1 H). MS (ESI+) m/z=270/272.

### Intermediate 55

### [3-(Ethoxycarbonyl)pyrazolo[1,5-α]pyrimidin-6-yl]boronic acid

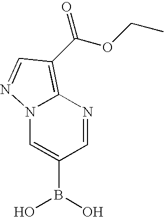

A solution of ethyl 6-bromopyrazolo[1,5-α]pyrimidine-3-carboxylate (INTERMEDIATE 54, 143 mg, 0.529 mmol) in toluene/H₂O 4:1 (5 mL) was degassed by bubbling N₂ through the solution. Bis(pinacolato)diboron (162 mg, 0.640 mmol), KOAc (156 mg, 1.60 mmol) and bis(triphenylphosphine)palladium(II) chloride (18.4 mg, 0.0265 mmol) were added and the mixture stirred at 90° C. under N₂ overnight. The reaction mixture was acidified with 1M HCl and extracted with EtOAc. The organic layer was concentrated, the residue dissolved in EtOAc and extracted with 1M NaOH. The aqueous layer was acidified and re-extracted with EtOAc. The organic layer was concentrated to give the title compound (70.3 mg, 57%) as a brown solid. The material was used without further purifications. ¹H NMR (400 MHz, CDCl₃) δ ppm 1.36 (t, J=7.08 Hz, 3 H) 4.39 (q, J=7.08 Hz, 2 H) 8.53 (s, 1 H) 8.92 (d, J=1.71 Hz, 1 H) 8.99 (d, J=1.71 Hz, 1 H).

### Intermediate 56

### 6-(Dihydroxyboryl)pyrazolo[1,5-α]pyrimidine-3-carboxylic acid

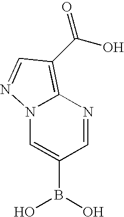

[3-(ethoxycarbonyl)pyrazolo[1,5-α]pyrimidin-6-yl]boronic acid (INTERMEDIATE 55, 1.0 g, 4.3 mmol) was treated with 1M LiOH (12.7 mL) and the solution heated at 65° C. for 1 h. The reaction mixture was cooled to r.t. and 1M HCl (12.7 mL) was added. The precipitated product was filtered off, washed with 1M HCl and H₂O and dried to give the title compound (0.74 g, 83%), which was directly used without further purification.

### Intermediate 57

### (3-{[(2-Amino-2-oxoethyl)amino]carbonyl}pyrazolo[1,5-α]pyrimidin-6-yl)boronic acid

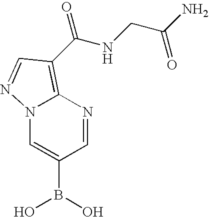

A solution of 6-(dihydroxyboryl)pyrazolo[1,5-α]pyrimidine-3-carboxylic acid (INTERMEDIATE 56, 730 mg, 3.5 mmol) in DMF (10 mL) was treated with triethylamine (2.09 ml, 14.4 mmol), TBTU (1.37 g, 4.20 mmol) and glycinamide hydrochloride (0.47 g, 4.2 mmol) and the reaction mixture was stirred at r.t. for 1.5 h. CH₃CN (40 mL) was added, the precipitated product filtered off, washed with CH₃CN and dried to give the title compound (861 mg, 94%). MS (ESI+) for C₉H₁₀BN₅O₄ m/z 264 (M+H)⁺.

### Example 53

General Procedure C

### N-(2-Amino-2-oxoethyl)-6-(2,5-dichlorobenzyl)pyrazolo[1,5-α]pyrimidine-3-carboxamide

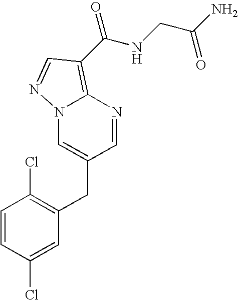

A mixture of (3-{[(2-amino-2-oxoethyl)amino]carbonyl}pyrazolo[1,5-α]pyrimidin-6-yl)boronic acid (INTERMEDIATE 57, 25 mg, 0.095 mmol), 2-(bromomethyl)-1,4-dichlorobenzene (25 mg, 0.10 mmol) and Pd(PPh₃)₄ (11 mg, 0.10 mmol) in dioxane (1 mL) was treated with a solution of K₂CO₃ (29 mg, 0.21 mmol) in H₂O (250 μL). The mixture was stirred at 90° C. for 3 h, cooled to r.t. and treated with HOAc (12 μl, 0.21 mmol). The reaction mixture was filtered and purified by preparative HPLC (ACE C8, 0.1% TFA—CH₃CN). Yield: 4.3 mg, 12%. MS (ESI+) calcd for C₁₆H₁₃Cl₂N₅O₂ 377.0446, found 377.0446.

### Example 54

### N-(2-Amino-2-oxoethyl)-6-15-chloro-2-(trifluoromethyl)benzyl]pyrazolo[1,5-α]-pyrimidine-3-carboxamide

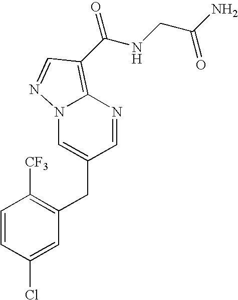

The title product was prepared according to General procedure C, using 2-(bromomethyl)-4-chloro-1-(trifluoromethyl)benzene (29 mg, 0.10mmol) as the benzylic halide. Yield: 3.2 mg, 8%. MS (ESI+) calcd for C₁₇H₁₃ClF₃N₅O₂ 411.0709, found 411.0708.

### Example 55

### N-(2-Amino-2-oxoethyl)-6-[2-chloro-5-(trifluoromethyl)benzyl]pyrazolo[1,5-α]-pyrimidine-3-carboxamide

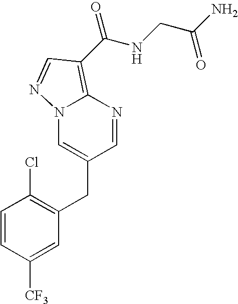

The title product was prepared according to General procedure C, using 2-(bromomethyl)-1-chloro-4-(trifluoromethyl)benzene (29 mg, 0.10 mmol) as the benzylic halide. (Yield: 10 mg, 25%. MS (ESI+) calcd for C₁₇H₁₃CIF₃N₅O₂ 411.0709, found 411.0711.

### Example 56

### N-(2-Amino-2-oxoethyl)-6-(2,3-dichlorobenzyl)pyrazolo[1,5-α]pyrimidine-3-carboxamide

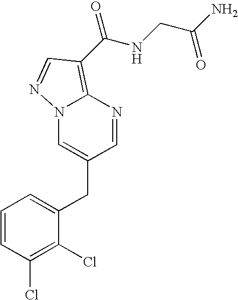

The title product was prepared according to General procedure C, using 1-(bromomethyl)-2,3-dichlorobenzene (25 mg, 0.10 mmol) as the benzylic halide. Yield: 5.5 mg, 15%. MS (ESI+) calcd for C₁₆H₁₃Cl₂N₅O₂ 377.0446, found 377.0449.

### Example 57

### N-(2-Amino-2-oxoethyl)-6-(4-chloro-2-fluorobenzyl)pyrazolo[1,5-α]pyrimidine-3-carboxamide

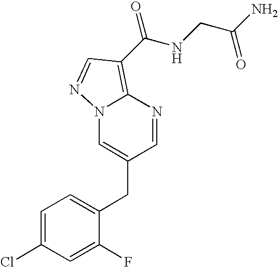

The title product was prepared according to General procedure C, using 1-(bromomethyl)-4-chloro-2-fluorobenzene (23 mg, 0.10 mmol) as the benzylic halide. Yield: 7.7 mg, 22%. MS (ESI+) calcd for C₁₆H₁₃ClFN₅O₂ 361.0741, found 361.0746.

### Example 58

### N-(2-Amino-2-oxoethyl)-6-(5-chloro-2-fluorobenzyl)pyrazolo[1,5-α]pyrimidine-3-carboxamide

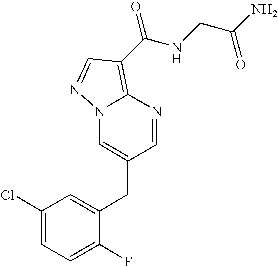

The title product was prepared according to General procedure C, using 2-(bromomethyl)-4-chloro-1-fluorobenzene (23 mg, 0.10 mmol) as the benzylic halide. Yield: 10.7 mg, 31%. MS (ESI+) calcd for C₁₆H₁₃ClFN₅O₂ 361.0741, found 361.0744.

### Example 59

### N-(2-Amino-2-oxoethyl)-6-[2-methyl-5-(trifluoromethyl)benzyl]pyrazolo[1,5-α]-pyrimidine-3-carboxamide

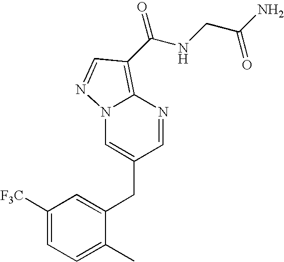

The title product was prepared according to General procedure C, using 2-(chloromethyl)-1-methyl-4-(trifluoromethyl)benzene (22 mg, 0.10 mmol) as the benzylic halide. Yield: 9.4 mg, 25%. MS (ESI+) calcd for C₁₈H₁₆F₃N₅O₂ 391.1256, found 391.1256.

### Example 60

### N-(2-Amino-2-oxoethyl)-6-(2,4-dichlorobenzyl)pyrazolo[1,5-α]pyrimidine-3-carboxamide

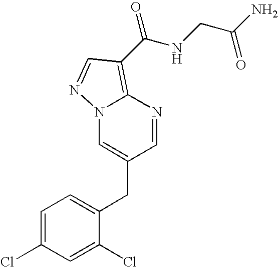

The title product was prepared according to General procedure C, using 2,4-dichloro-1-(chloromethyl)benzene (20 mg, 0.10 mmol) as the benzylic halide. Yield: 14 mg, 39%. MS (ESI+) calcd for C₁₆H₁₃Cl₂N₅O₂ 377.0446, found 377.0450.

### Biological Examples

Background to Assay Methodology

Several assay methods for measuring stearoyl-CoA desaturase activity have been described in the literature. Thin layer chromatography, gas chromatography or HPLC methods are commonly used for separation of substrates and products, e.g. stearoyl-CoA and oleyl-CoA, following the enzymatic reaction [see e.g. Henderson & Henderson (1992) In Lipid analysis: A practical approach. Oxford University Press, New York and Tokyo, editor S. Hamilton, pages 65-111]. However, these assays are time-consuming and not amenable to higher throughputs. Spectrophotometric assays in which the SCD activity is followed indirectly by measuring the reoxidation of reduced cytochrome B5 could be applied [Strittmatter (1978) Purification of cytochrome B5. Meth. Enzymol. 52, 97-101]although the fast reoxidation rate complicates the automation of such assays. It may be possible to achieve a reasonable throughput given auto-injectors and fast readers or alternative systems that allow parallel processing of multiple samples, but spectroscopic assays based on near-UV wavelength measurements also have the added disadvantage of being prone to artifacts by colored and autofluorescent compounds.

Another measure of SCD activity was introduced by Talamo and Bloch in 1969 [Talamo & Bloch (1969) Anal. Biochem. 29, 300-304]. This method is based on the quantification of a second product of the desaturase reaction, i.e. the water molecule that is released in the desaturase reaction. The quantification is based on the use of a long chain acyl-CoA substrate, e.g. stearoyl-CoA, that is specifically labeled with tritium in positions 9 and 10 of the carbon chain such that the released water is also tritiated ([³H]—H₂O). The remaining [³H]-stearoyl-CoA as well as the product [³H]-oleyl-CoA must then be separated from the solution before the tritiated water content can be measured by means of liquid scintillation. Talamo and Bloch acid precipitated the long chain acyl-CoAs followed by filtration to achieve this separation, but this separation can also be achieved by means of centrifugation instead of filtration [Johnson & Guhr (1971) Lipids 6, 78-84]. An alternative procedure that involves precipitation by ethanol and activated charcoal followed by centrifugation have also been described [Shanklin and Somerville (1991) Proc. Natl. Acad. Sci. USA 88, 2510-2514]. Based on these studies it is clear that near perfect separation is required for optimal assay performance. When applying this assay it is important to recognize that the apparent desaturation rate is impacted by isotope effects as described by Johnson and Gurr in 1971 [Johnson & Guhr (1971) Lipids 6, 78-84]. Thus whereas the assay serves as an excellent measure of relative SCD activity it must be calibrated using other methods when absolute measures of enzyme activity are needed. The pros and cons of this assay have also been summarized in the literature [Gurr & Robinson (1972) Anal. Biochem. 47, 146-156].

An abundant source of stearoyl-CoA desaturase activity can be found in microsomal preparations from the liver of rats that have been subjected to a fasting-refeeding procedure on a low fat/high carbohydrate diet [reviewed in Ntambi (1999) J. Lipid Res. 40, 1549-1558]. However, microsomal preparations are not a pure source of SCD activity and this means that the added stearoyl-CoA substrate is subject also to other enzymatic processes. It is therefore essential to include reagents that allow regeneration of the stearoyl-CoA substrate as described by Bertram and Erwin [Bertram & Erwin (1981 J. Protozool. 8, 127-131].

The tritium release assay for the measurement of SCD activity is thus well documented in the literature. Descriptions on how these finding have been used to produce standard screening assays in 96-well plates are also available [Brownlie, Hayden, Attie, Ntambi, Gray-Keller, & Miyazaki (2001) WO 01/62954; Wu, Gallipoli, Gallagher, & Gardell (2004) WO 2004/04776]. We have adopted the tritium release assay to a 384-well format to improve throughput even further. The assay is based on the findings made decades ago and hence is available to anyone skilled in the art of assay automation and high throughput screening.

Description of Screening Assay for the Identification and Characterization of Test Compounds that Inhibit Stearoyl-CoA Desaturase Activity

Microsomal preparations were prepared from the livers of Male Sprague-Dawley rats that had been fasted and then refed a low fat/high carbohydrate diet. The preparation of microsomes was adopted from Seifried and Gaylor [Seifried & Gaylor (1976) J. Biol. Chem. 251, 7468-7473]. Confirmation of compound activity on human material was made based on microsomal preparations from HepG2 cells. All other reagents were purchased from commercial sources. The assay was run in 96 or 384-well microtiter plates by consecutive additions of a test compound solution, a microsomal preparation solution and a substrate containing solution. The final concentrations of all reagents in a total assay volume of 40 μl per well (in the 384-well plate format) were:
0.11 μM [³H]-stearoyl-CoA
50 nM stearoyl-CoA
0.032 mg/ml rat liver microsomes (total protein content)
2 mM NADH
220 mM sucrose
44 mM NaH₂PO₄ pH adjusted to 6.8
130 mM KCl
1.3 mM GSH
0.05 mM CoA
0.1% BSA
0.29 mM nicotine amide
15 mM NaF
1.1 mM ATP
4.9 mM MgCl2
0.002% Tween-20
A test compound at various concentrations (which also adds 0.5-2% DMSO to the final solution)

The test compounds were pre-incubated for 20 minutes with the microsomal preparation prior to starting the reaction by the addition of substrate. The enzymatic reaction was allowed to proceed for 20 minutes and then optionally slowed by an addition of 40 μl of a 2% DMSO solution in water containing a known inhibitor of SCD activity. The solutions were mixed and then 70 μl of the total 80 μl were transferred to a filter plate containing predispensed activated charcoal. The plate was then centrifuged and the filtrate collected in a collector plate to which 40 μl of Optiphase Supermix was added per well. Following an 18 h equilibration time at room temperature the plate was read in a Trilux MicroBeta (two minutes counting time per well). On all assay occasions controls were included on each plate to define the values for uninhibited and fully inhibited reactions and these values were used to calculate the % inhibition of the enzymatic reaction at any given compound concentration. The inhibitory potency or IC₅₀ values of test compounds on SCD activity were defined by applying the same assay in the presence of sub-nM to sub-mM compound concentrations. Examples included herein have IC₅₀ values in the range of 1 nM to 1 μM (see Table I for exemplary data) as measured using the above described assay or in the equivalent assay in a 96-well microtiter plate format.

TABLE I
IC₅₀ values for SCD inhibition

| Example | IC₅₀(μM) |
|---|---|
| 25 | 0.027 |
| 38 | 0.14 |
| 39 | 0.61 |
| 48 | 0.091 |
| 50 | 0.48 |
| 56 | 0.022 |

1. A compound of formula (I), 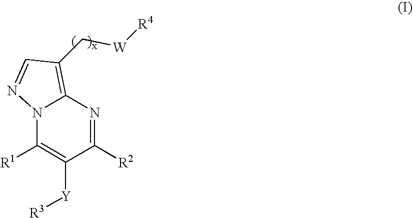
and pharmaceutically acceptable salts, solvates, hydrates, geometrical isomers, racemates, tautomers, optical isomers, or N-oxides thereof, wherein:
x is 0 or 1;
W is selected from the group consisting of a direct bond, —C(O)N(R⁵)—, —N(R⁵)C(O)—, —C(O)O—, —OC(O)—, —O—, —N(R⁵)C(O)N(R⁵)—, and —N(R⁵)—, wherein each R⁵ is independently hydrogen, C₁₋₃-alkyl, or C₁₋₄-alkoxy-C₂₋₄-alkyl;
R¹ and R² are independently selected from the group consisting of hydrogen, C₁₋₃-alkyl and C₁₋₃-fluoroalkyl, provided that at least one of R¹ and R² is hydrogen;
Y is selected from the group consisting of —S—, —O—, —N— and C₁₋₃-alkylene, wherein C₁₋₃-alkylene is optionally monosubstituted with hydroxy or oxo, or is partly or fully fluorinated;
R³ is aryl or heteroaryl, said aryl or heteroaryl residue being optionally substituted in one or more positions with a substituent independently selected from:
(a) halogen,
(b) C ₁₋₆-alkyl,
(c) C₁₋₆-alkoxy,
(d) fluoro-C₁₋₃-alkyl,
(e) fluoro-C₁₋₃-alkoxy,
(f) C₃₋₇-cycloalkyl,
(g) C₃₋₇-cycloalkoxy,
(h) methylenedioxy,
(i) hydroxy-C₁₋₃-alkyl,
(j) cyano,
(k) hydroxy,
(l) C₁₋₆-alkylthio,
(m) fluoro-C₁₋₆-alkylthio,
(n) C₁₋₆-alkylsulfonyl,
(o) aryl-C₁₋₃-alkoxy, wherein aryl is optionally substituted in one or two positions with a substituent selected from halogen, methoxy, ethoxy, methyl, ethyl and trifluororomethyl;
R⁴ is selected from the group consisting of C₁₋₄-alkoxy-C₂₋₆-alkyl, hydroxy-C₁₋₆-alkyl, C₁₋₄-alkylthio-C₂₋₆-alkyl, cyano-C₁₋₆-alkyl, heteroarylamino-C₂₋₆-alkyl, heterocyclylamino-C₂₋₆-alkyl, heterocyclyl-C₁₋₆-alkyl, aryl-C₁₋₄-alkoxy-C₂₋₄-alkyl, dihydroxy-C₃₋₄-alkoxy-C₂₋₄-alkyl, cyano-C₁₋₄-alkoxy-C₂₋₄-alkyl, hydroxy-C₂₋₄-alkoxy-C₂₋₄-alkyl, aminocarbonyl-C₁₋₄-alkoxy-C₂₋₄-alkyl, C₁ ₄-alkoxy-C₂₋₄-alkoxy-C₂₋₄-alkyl, hydroxy-C₂₋₄-alkoxy-C₂₋₄-alkoxy-C₂₋₄-alkyl, C₂₋₄-alkenyloxy-C₂₋₄-alkyl, C_{1 4}-alkylaminocarbonyl-C₁₋₄-alkoxy-C₂₋₄-alkyl, di-(C₁₋₂-alkyl)aminocarbonyl-C₁₋₄-alkoxy-C₂₋₄-alkyl, aryl, aryl-C₁₋₆-alkyl, heteroaryl and heteroaryl-C₁₋₆-alkyl, wherein any aryl or heteroaryl residue can be optionally substituted with one or more substituents R⁸; or
R⁴ is C₁₋₆-alkylene-V—R⁶;
V is selected from the group consisting of —C(O)N(R⁷)—, —N(R⁷)C(O)—, —C(O)O)—, —OC(O)—, —C(O)—, —N(R⁷)C(O)O—, —OC(O)N(R⁷)—, —N(R⁷)C(O)N(R⁷)—, —S—, —S(O)—, —S(O)₂—, —S(O)N(R⁷)—, —N(R⁷)S(O)—, —S(O)₂N(R⁷)— and —N(R⁷)S(O)₂—;
each R⁶ and each R⁷ are independently selected from the group consisting of hydrogen, C₁₋₅-alkyl, C₃₋₆-cycloalkyl (optionally substituted with oxo), C₃₋₆-cycloalkyl-C₁₋₄-alkyl, hydroxy-C₁₋₄-alkyl, C₂₋₄-alkynyl, fluoro-C₁₋₅-alkyl, aryl, aryl-C₁₄-alkyl, heteroaryl, and heteroaryl-C₁₋₄-alkyl, wherein any aryl or heteroaryl residue can be optionally substituted with one or more substituents R⁸;
provided that when V is selected from —S(O)—, —S(O)₂—, —C(O)—, —N(R⁷)C(O)O—, —N(R⁷)S(O)—, or —N(R⁷)S(O)₂—, then R⁶ is not hydrogen;
R⁸ is independently selected from the group consisting of:
(a) C ₁₋₄-alkylsulfonyl,
(b) C₁₋₄-alkylsulfinyl,
(c) C₁₋₄-alkylthio,
(d) hydroxy-C₂₋₄-alkylsulfonyl,
(e) trifluoromethylsulfonyl,
(f) —S(O)₂NR⁹R⁹,
(g) C₁₋₄-alkylsulfonamido,
(h) C₂₋₄-acylamino,
(i) C₂₋₄-acylaminomethyl,
(j) —C(O)NR⁹R⁹,
(k) —CH₂—C(O)NR⁹R⁹
(l) —NHC(O)OCH₃,
(m) C₁₋₄-alkoxy,
(n) C₃₋₅-cycloalkyloxy,
(o) —CN,
(p) —OH,
(q) C₁₋₆-alkyl
(r) hydroxy-C₁₋₂-alkyl,
(s) cyano-C₁₋₂-alkyl,
(t) C₁₋₂-alkoxy-C₁₋₂-alkyl, and
(u) halogen;
R⁹ is each independently selected from the group consisting of:
(a) hydrogen,
(b) C₁₋₃-alkyl,
(c) hydroxy-C₂₋₄-alkyl,
(d) dihydroxy-C₂₋₄-alkyl,
(e) cyano-C₁₋₃-alkyl,
(f) C₁₋₂-alkoxy-C₂₋₄-alkyl, and
(g) aminocarbonyl-C₁₋₂-alkyl.

2. A compound according to claim 1, wherein R¹ is methyl and R² is H.

3. A compound according to claim 1, wherein R¹ is H and R² is methyl.

4. A compound according to claim 1, wherein R¹ and R² are each H.

5. A compound according to claim 1, wherein x is 0 and W is selected from —C(O)NH—, —NHC(O)—, —C(O)O— and —NHC(O)NH—.

6. A compound according to claim 1, wherein Y is methylene, 1,1-ethylene or —S—.

7. A compound according to claim 1, wherein R³ is phenyl.

8. A compound according to claim 1, wherein R³ is selected from the group consisting of phenyl, 3-bromophenyl, 4-bromophenyl, 3-trifluoromethylphenyl, 3-trifluoromethoxyphenyl, 3,4-dichlorophenyl, 2,3-dichlorophenyl, 2,4-dichlorophenyl, 2,5-dichlorophenyl, 4-chloro-2-fluorophenyl, 3-chloro-4-fluorophenyl, 5-chloro-2-fluorophenyl, 2-methyl-5-trifluoromethylphenyl, 4-chloro-3-trifluoromethylphenyl, 4-fluoro-3-trifluoromethylphenyl, 4-chloro-3-trifluoromethoxyphenyl, 4-fluoro-3-trifluoromethoxyphenyl, 5-chloro-2-trifluoromethylphenyl, and 2-chloro-5-trifluoromethylphenyl.

9. A compound according to claim 1, wherein R⁴ is selected from the group consisting of 2-methoxyethyl, 2-hydroxyethyl, 3-methoxypropyl, 3-hydroxypropyl, 2-(2-hydroxyethoxy)ethyl, 2-(2-aminocarbonylethoxy)ethyl, cyanomethyl, 2-(2-cyanoethoxy)ethyl, 2-(2-hydroxy-2-methylpropoxy)ethyl, 2-(formylamino)-ethyl, 2-(acetylamino)ethyl, 2-(propionylamino)ethyl, 2-(ethynylcarbonylamino)-ethyl, aminocarbonylmethyl, methylaminocarbonylmethyl, 2-(aminocarbonyl)ethyl, 2-(hydroxymethylcarbonylamino)ethyl, 2-(methylsulfinyl)ethyl, 2-(methylsulfonyl)-ethyl, 2-(dimethylamino)-2-oxoethyl, 2-(benzyloxy)ethyl, tetrahydrofuran-2-yl-methyl, 2-[(1H-pyrrol-2-ylcarbonyl)amino]ethyl, 2-furylmethyl, 2-(2-furyl)ethyl, 2-[(2-furylmethyl)thio]ethyl, 2-(pyridin-2-ylamino)ethyl, 6-methoxypyridin-3-yl, 2-[(pyrazin-2-ylcarbonyl)amino]ethyl, 2-(isonicotinoylamino)ethyl, pyridin-3-yl, [6-(hydroxymethyl)pyridin-2-yl]methyl and 2-(2,3-dihydroxypropoxy)ethyl.

10. A compound according to claim 1, which is selected from the group consisting of:
tert-butyl[2-({[6-(3,4-dichlorobenzyl)pyrazolo[1,5-α]pyrimidin-3-yl]carbonyl}-amino)ethyl]carbamate
6-(3,4-dichlorobenzyl)-N-{2-[(pyrazin-2-ylcarbonyl)amino]ethyl}pyrazolo[1,5-α]-pyrimidine-3-carboxamide;
6-(3,4-dichlorobenzyl)-N-[2-(methylsulfinyl)ethyl]pyrazolo[1,5-α]pyrimidine-3-carboxamide;
6-(3,4-dichlorobenzyl)-N-[2-(methylsulfonyl)ethyl]pyrazolo[1,5-α]pyrimidine-3-carboxamide;
6-(3,4-dichlorobenzyl)-N-[2-(dimethylamino)-2-oxoethyl]pyrazolo[1,5-α]-pyrimidine-3-carboxamide;
6-(3,4-dichlorobenzyl)-N-[2-(methylamino)-2-oxoethyl]pyrazolo[1,5-α]-pyrimidine-3-carboxamide;
N-[2-(benzyloxy)ethyl]-6-(3,4-dichlorobenzyl)pyrazolo[1,5-α]pyrimidine-3-carboxamide;
6-(3,4-dichlorobenzyl)-N-(3-methoxypropyl)pyrazolo[1,5-α]pyrimidine-3-carboxamide;
6-(3,4-dichlorobenzyl)-N-(3-hydroxypropyl)pyrazolo[1,5-α]pyrimidine-3-carboxamide;
6-(3,4-dichlorobenzyl)-N-(tetrahydrofuran-2-ylmethyl)pyrazolo[1,5-α]pyrimidine-3-carboxamide;
6-(3,4-dichlorobenzyl)-N-[2-(isonicotinoylamino)ethyl]pyrazolo[1,5-α]-pyrimidine-3-carboxamide;
6-(3,4-dichlorobenzyl)-N-[2-(pyridin-2-ylamino)ethyl]pyrazolo[1,5-α]pyrimidine-3-carboxamide;
6-(3,4-dichlorobenzyl)-N-[2-(2-furyl)ethyl]pyrazolo[1,5-α]pyrimidine-3-carboxamide;
6-(3,4-dichlorobenzyl)-N-{2-[(2-furylmethyl)thio]ethyl}pyrazolo[1,5-α]-pyrimidine-3-carboxamide;
N-(3-amino-3-oxopropyl)-6-(3,4-dichlorobenzyl)pyrazolo[1,5-α]pyrimidine-3-carboxamide;
N-(2-amino-2-oxoethyl)-6-(3,4-dichlorobenzyl)pyrazolo[1,5-α]pyrimidine-3-carboxamide;
6-(4-bromobenzyl)-N-[2-(propionylamino)ethyl]pyrazolo[1,5-α]pyrimidine-3-carboxamide;
6-(4-bromobenzyl)-N-{2-[(1H-pyrrol-2-ylcarbonyl)amino]ethyl}pyrazolo[1,5-α]-pyrimidine-3-carboxamide;
6-(4-bromobenzyl)-N-(2-hydroxyethyl)pyrazolo[1,5-α]pyrimidine-3-carboxamide;
6-(4-bromobenzyl)-N-[2-(2,3-dihydroxypropoxy)ethyl]pyrazolo[1,5-α]-pyrimidine-3-carboxamide;
6-(4-bromobenzyl)-N-(2-methoxyethyl)pyrazolo[1,5-α]pyrimidine-3-carboxamide;
N-[2-(3-amino-3-oxopropoxy)ethyl]-6-(4-bromobenzyl)pyrazolo[1,5-α]-pyrimidine-3-carboxamide;
6-(4-bromobenzyl)-N-[2-(2-cyanoethoxy)ethyl]pyrazolo[1,5-α]pyrimidine-3-carboxamide;
6-(4-bromobenzyl)-N-[2-(2-hydroxy-2-methylpropoxy)ethyl]pyrazolo[1,5-α]-pyrimidine-3-carboxamide;
6-(4-bromobenzyl)-N-[2-(formylamino)ethyl]pyrazolo[1,5-α]pyrimidine-3-carboxamide;
6-(4-bromobenzyl)-N-[2-(glycoloylamino)ethyl]pyrazolo[1,5-α]pyrimidine-3-carboxamide;
6-(3-chloro-4-fluorobenzyl)-N-{[6-(hydroxymethyl)pyridin-2-yl]methyl}-pyrazolo[1,5-α]pyrimidine-3-carboxamide;
N-(2-amino-2-oxoethyl)-6-(3-chloro-4-fluorobenzyl)pyrazolo[1,5-α]pyrimidine-3-carboxamide;
N-(3-amino-3-oxopropyl)-6-(3-chloro-4-fluorobenzyl)pyrazolo[1,5-α]pyrimidine-3-carboxamide;
6-(3-chloro-4-fluorobenzyl)-N-[2-(2-cyanoethoxy)ethyl]pyrazolo[1,5-α]-pyrimidine-3-carboxamide;
6-[4-chloro-3-(trifluoromethoxy)benzyl]-N-(2-hydroxyethyl)pyrazolo[1,5-α]-pyrimidine-3-carboxamide;
N-(3-amino-3-oxopropyl)-6-[4-chloro-3-(trifluoromethoxy)benzyl]pyrazolo[1,5-α]pyrimidine-3-carboxamide;
N-(2-amino-2-oxoethyl)-6-[4-chloro-3-(trifluoromethyl)benzyl]pyrazolo[1,5-α]-pyrimidine-3-carboxamide;
N-[2-(acetylamino)ethyl]-6-[4-fluoro-3-(trifluoromethyl)benzyl]pyrazolo[1,5-α]-pyrimidine-3-carboxamide;
N-(2-amino-2-oxoethyl)-6-[4-fluoro-3-(trifluoromethyl)benzyl]pyrazolo[1,5-α]-pyrimidine-3-carboxamide;
N-(3-amino-3-oxopropyl)-6-{1-[3-(trifluoromethyl)phenyl]ethyl}pyrazolo[1,5-α]-pyrimidine-3-carboxamide;
6-benzyl-N-[2-(2-hydroxyethoxy)ethyl]-5-methylpyrazolo[1,5-α]pyrimidine-3-carboxamide;
6-[4-fluoro-3-(trifluoromethoxy)benzyl]-N-[2-(2-hydroxyethoxy)ethyl]-5-methyl-pyrazolo[1,5-α]pyrimidine-3-carboxamide;
6-benzyl-N-[2-(2-hydroxyethoxy)ethyl]-7-methylpyrazolo[1,5-α]pyrimidine-3-carboxamide;
N-[2-(2-hydroxyethoxy)ethyl]-7-methyl-6-[3-(trifluoromethyl)benzyl]pyrazolo-[1,5-α]pyrimidine-3-carboxamide;
N-(3-amino-3-oxopropyl)-7-methyl-6-[3-(trifluoromethoxy)benzyl]pyrazolo-[1,5-α]pyrimidine-3-carboxamide;
N-[2-(acetylamino)ethyl]-6-[4-chloro-3-(trifluoromethoxy)benzyl]-7-methyl-pyrazolo[1,5-α]pyrimidine-3-carboxamide;
6-[4-chloro-3-(trifluoromethoxy)benzyl]-N-[2-(2-hydroxyethoxy)ethyl]-7-methyl-pyrazolo[1,5-α]pyrimidine-3-carboxamide;
N-[2-(acetylamino)ethyl]-6-[4-fluoro-3-(trifluoromethoxy)benzyl]-7-methyl-pyrazolo[1,5-α]pyrimidine-3-carboxamide;
6-[4-fluoro-3-(trifluoromethyl)benzyl]-N-(6-methoxypyridin-3-yl)pyrazolo-[1,5-α]pyrimidine-3-carboxamide;
6-[4-fluoro-3-(trifluoromethyl)benzyl]-N-pyridin-3-ylpyrazolo[1,5-α]pyrimidine-3-carboxamide;
2-(acetylamino)ethyl 6-[4-fluoro-3-(trifluoromethyl)benzyl]pyrazolo[1,5-α]-pyrimidine-3-carboxylate;
2-amino-2-oxoethyl 6-[4-fluoro-3-(trifluoromethyl)benzyl]pyrazolo[1,5-α]-pyrimidine-3-carboxylate;
2-(2-hydroxyethoxy)ethyl 6-[4-fluoro-3-(trifluoromethyl)benzyl]pyrazolo[1,5-α]-pyrimidine-3-carboxylate;
N-(2-amino-2-oxoethyl)-6-{[3-(trifluoromethyl)phenyl]thio}pyrazolo[1,5-α]-pyrimidine-3-carboxamide;
2-cyano-N-[6-(3,4-dichlorobenzyl)pyrazolo[1,5-α]pyrimidin-3-yl]acetamide;
N-[6-(3,4-dichlorobenzyl)pyrazolo[1,5-α]pyrimidin-3-yl]-N′-(2-furylmethyl)urea;
N-(2-amino-2-oxoethyl)-6-(2,5-dichlorobenzyl)pyrazolo[1,5-α]pyrimidine-3-carboxamide;
N-(2-amino-2-oxoethyl)-6-[5-chloro-2-(trifluoromethyl)benzyl]pyrazolo[1,5-α]-pyrimidine-3-carboxamide;
N-(2-amino-2-oxoethyl)-6-[2-chloro-5-(trifluoromethyl)benzyl]pyrazolo[1,5-α]-pyrimidine-3-carboxamide;
N-(2-amino-2-oxoethyl)-6-(2,3-dichlorobenzyl)pyrazolo[1,5-α]pyrimidine-3-carboxamide;
N-(2-amino-2-oxoethyl)-6-(4-chloro-2-fluorobenzyl)pyrazolo[1, 5-α]pyrimidine-3-carboxamide;
N-(2-amino-2-oxoethyl)-6-(5-chloro-2-fluorobenzyl)pyrazolo[1, 5-α]pyrimidine-3-carboxamide;
N-(2-amino-2-oxoethyl)-6-[2-methyl-5-(trifluoromethyl)benzyl]pyrazolo[1,5-α]-pyrimidine-3-carboxamide; and
N-(2-amino-2-oxoethyl)-6-(2,4-dichlorobenzyl)pyrazolo[1,5-α]pyrimidine-3-carboxamide.

11. A compound according to claim 2 wherein x is 0 and W is selected from —C(O)NH—, —NHC(O)—, —C(O)O— and —NHC(O)NH— and Y is methylene, 1,1-ethylene or —S—.

12. A compound according to claim 3 wherein x is 0 and W is selected from —C(O)NH—, —NHC(O)—, —C(O)O— and —NHC(O)NH— and Y is methylene, 1,1-ethylene or —S—.

13. A compound according to claim 4 wherein x is 0 and W is selected from —C(O)NH—, —NHC(O)—, —C(O)O— and —NHC(O)NH— and Y is methylene, 1,1-ethylene or —S—.

14. A compound according to claim 1 wherein x is 0 and W is selected from —C(O)NH—, —NHC(O)—, —C(O)O— and —NHC(O)NH— and Y is methylene, 1,1-ethylene or —S—.

15. A method for the modulation of stearoyl-CoA desaturase activity which comprises administering to a mammal, including man, in need of such treatment an effective amount of a compound according to claim 1.

16. A method for the modulation of plasma lipid levels which comprises administering to a mammal, including man, in need of such treatment an effective amount of a compound according to claim 1.

17. A method for treatment or prevention of cardiovascular diseases, obesity, non-insulin-dependent diabetes mellitus, hypertension, metabolic syndrome, neurological diseases, immune disorders, cancer, essential fatty acid deficiency, eczema, acne, psoriasis, rosacea or other skin conditions caused by lipid imbalance, or for treatment of excessive hair growth, which comprises administering to a mammal, including man, in need of such treatment an effective amount of a compound according to claim 1.

18. A pharmaceutical formulation comprising a compound according to claim 1 as active ingredient, in combination with a pharmaceutically acceptable diluent or carrier.

19. A pharmaceutical formulation according to claim 18, further comprising an additional therapeutic agent.
